(19) United States
(12) Patent Application Publication
(10) Pub. No.: US 2008/0050814 A1
(43) Pub. Date: Feb. 28, 2008

(54) Procurement, isolation and cryopreservation of fetal placental cells

(75) Inventor: Julie G. Allickson, Odessa, FL (US)

Correspondence Address: JOHN W. GOLDSCHMIDT, JR. ESQUIRE;DILWORTH PAXON LLP, 3200 MELLON BANK CENTER, 1735 MARKET STREET, PHILADELPHIA, PA, 19103, US

(73) Assignee: Cryo-Cell International, Inc., Oldsmar, FL (US)

(21) Appl. No.: 11/810,851

(22) Filed: Jun. 5, 2007

Related U.S. Application Data
(60) Provisional application No. 60/811,156, filed on Jun. 5, 2006.
(60) Provisional application No. 60/811,651, filed on Jun. 6, 2006.
(60) Provisional application No. 60/811,935, filed on Jun. 7, 2006.
(60) Provisional application No. 60/876,591, filed on Dec. 22, 2006.

(51) Int. Cl. C12N 5/02; C12M 3/00; C12N 5/08; C12M 3/08

(52) U.S. Cl. 435/366; 435/307.1; 435/374; 435/378

(57) ABSTRACT

Methods, processes and systems for procuring, isolating and cryopreserving fetal placental cells are provided. A population of fetal placental cells is also provided.

### CROSS REFERENCE TO RELATED APPLICATIONS

This application claims the priorities of U.S. Provisional Patent Application Ser. No. 60/811,156, filed Jun. 5, 2006; U.S. Provisional Patent Application Ser. No. 60/811,651, filed Jun. 6, 2006; U.S. Provisional Patent Application Ser. No. 60/811,935, filed Jun. 7, 2006; and U.S. Provisional Patent Application Ser. No. 60/876,591, filed Dec. 22, 2006, each entitled “Procurement, Isolation and Cryopreservation of Placental Stem Cells,” the entireties of which are incorporated herein by reference.

### FIELD OF THE INVENTION

The invention relates to methods, processes and systems for procuring and processing placental tissue obtained from a whole placenta; for isolating, collecting and cryopreserving a population of fetal cells obtained from the procured placental tissue; and for selecting, culturing and cryopreserving placental cells.

### BACKGROUND OF THE INVENTION

The human placenta develops from cells of fetal and maternal origin during implantation of a fetus into a uterus. Placental tissue is a combination of fetal cells that form fetal placental tissue and maternal cells that form maternal placental tissue. The fetal tissue and maternal tissue are affixed together with connective tissue to form the diversely functional placenta. Many of the fetal cells and the maternal cells of the placenta are characteristically capable of proliferation and differentiation. The fetal cells present in the placenta include, but are not limited to, fetal stem cells, hematopoietic cells, epithelial cells, mesenchymal or fibroblast-like cells, trophoblast cells, and other progenitor cells. The maternal cells present in the placenta include but are not limited to maternal mesenchymal or fibroblast-like cells, cells of the maternal immune system and other maternal cells.

Maternal cells and fetal cells coexist in the placenta throughout gestation. The fetal portion of a term placenta comprises the umbilical cord, the amniotic membrane on the cord, the amnion and the chorionic plate. During maturation of the placenta, the chorionic villi fuse with the amnion forming a seamless layer of amniochorion. The maternal portion comprises maternal decidual tissues on the opposite side of the umbilical cord and, also, the maternal blood that flows through the capillaries in the chorion. Maternal cells have been retrieved from a collection of maternal decidual tissue. Int'Anker P S et al. in 2004 harvested tissue from the decidua basalis and decidua parietalis, but did not reliably demonstrate the presence of maternal mesenchymal cells when subjected to functional viability assays (Int'Anker P S et al. Stem Cells 2004; 22:1338-1345). Takahashi et al. in 2002 (International Application Patent Number: PCT/JP03/03760) discussed the harvest and differentiation of maternal mesenchymal stem cells from placenta. The tissue was again harvested specifically from the maternal side of the organ and cells were isolated by explant or enzyme digest. These mesenchymal stem cells were unable to be differentiated into adipogenic or chondrogenic cell lineages.

Stem cells inherently possess the capability to undergo cellular division and cellular differentiation in vivo by way of control of cell-to-cell contact and intrinsic signals. Stem cells have been shown to be capable of dividing and differentiating in vitro into a variety of cells by controlling cell contact and intrinsic signals by stimulation with local environmental factors. It is recognized that stem cells may be obtained from several sources including a variety of adult tissues, such as bone marrow, and, also, embryonic tissues.

The transplantation of adult stem cells derived from bone marrow has been successfully used in treatment of human disease such as Fanconi's Anemia, Aplastic Anemia, Acute and Chronic Leukemias, Myeloproliferative Disorders, Myelodysplastic Syndromes, Lymphoproliferative Disorders and other malignancies. Alternative sources of bone marrow adult stem cells include peripheral blood progenitor cells, umbilical cord blood and mesenchymal stem cells harvested from these sources. However, there are several shortcomings associated with therapeutic use of adult stem cells. Adult stem cells have been shown to have limited efficacy such as slow growth and loss of pluripotency after several passages in culture.

Embryonic stem cells have demonstrated proliferative potential making them suitable for cellular therapy. However, human embryonic stem cells have been shown to produce teratomas. Additionally, the harvesting of stem cells from embryos poses ethical concerns due in part to the destruction of the growing embryo in the harvesting process.

Other sources of stem cells have been identified that overcome at least some of the issues associated with adult and embryonic stem cells.

One source is umbilical cord blood that contains human adult stem cells. Umbilical cord blood has proven to be a viable source of stem cells for several reasons. Umbilical cord blood is relatively easy to procure during delivery of a child and to process for cryopreservation. Umbilical cord blood provides a suitable source of stem cells capable of cellular division and differentiation into a variety of cell types. Stem cells derived from cord blood have been used in clinical settings to treat several known human disorders and diseases as an alternative to bone marrow. In particular, stem cells derived from cord blood have been successful in hematopoietic engraftment and hematopoietic reconstitution. A case study demonstrates the use of umbilical cord blood in treating spinal cord injuries. Additionally, stem cells derived from cord blood have been shown in animal models to reverse the effects of stroke and myocardial infarction. Further research is being conducted to identify additional therapeutic uses of stem cells derived from cord blood.

Advancements in research in the area of human stem cells have led researchers to alternative sources of stem cells including chorionic villi from the placenta and amniotic fluid. Collection of chorionic villi tissue and amniotic fluid for potential use in cell therapy involves the practice of methodologies that overcome the issues associated with collecting embryonic stem cells. Amniotic fluid containing fetal stem cells can be collected through the practice of amniocentesis. Amniocentesis involves the insertion of a fine needle through the abdomen of a pregnant woman into the uterus and amniotic sac. The needle is used to withdraw a desired amount of amniotic fluid containing human fetal stem cells from within the amniotic sac. In addition, chorionic villi are composed of cells that are of fetal origin and include fetal stem cells that can be collected through the practice of transcervical or transabdominal chorionic villi sampling (CVS). Chorionic villi are finger-like projections that emerge from the chorionic plate of the placenta and form part of the fetal portion of the placenta. Research indicates that selected human stem cells derived from amniotic fluid or chorionic villi are pluripotent and are, therefore, capable of differentiating into all three germ cell layers, highly proliferative, lacking significant immunogenicity due to no expression of HLA class II, and positive for the antigenic factor CD117 as described in United States Patent Application Publication No. US 2005/0124003.

Another source of viable stem cells is the human placenta post childbirth. Research indicates that fetal stem cells and other progenitor cells, including those cells from the maternal tissue, are present in the placenta. These stem cells and progenitor cells may be used in cellular therapy or tissue engineering. Research indicates that cells obtained from the placenta are capable of differentiating along osteogenic, adipogenic, chondrogenic, myogenic, endothelial, hepatic, neurologic and hematopoietic cell lineages. Other studies have examined the use of human umbilical cord tissue as a source of stem cells. Pluripotent placental cells are cells that may be used in regenerative medicine, tissue engineering and other therapies to treat human disorders.

Use of a human placenta as a source of fetal cells is beneficial for several reasons. The placenta is considered biohazardous medical waste after the birth of a baby. The placenta is typically discarded so the placenta is easily accessible for procurement of fetal cells from the placental tissue. Additionally, the process of procuring placental tissue after birth imparts no risk to the donor or the baby as may be associated with other fetal tissue collection techniques such as amniocentesis and CVS. Furthermore, any ethical concerns or considerations are alleviated by collecting placental tissue that is otherwise considered biohazardous medical waste.

One method for collecting placental stem cells is set forth in U.S. Pat. No. 7,045,148 to Hariri. The method appears to comprise collecting embryonic-like stem cells from a placenta, which has been treated to remove residual cord blood, by perfusing the drained placenta with an anticoagulant solution to flush out residual cells, collecting the residual cells and perfusion liquid from the drained placenta, and separating the embryonic-like cells expressing the cell marker CD34 from the residual cells and perfusion liquid. In order to practice the method, an exsanguinated placenta having the proximal umbilical cord clamped is recovered and transported to a laboratory for processing. At the laboratory, the umbilical cord is cannulated and connected to a perfusion manifold that pumps perfusion solution into the placenta. During the perfusion step, the placenta is used as a bioreactor for residual and stem cells present in the placenta, which are flushed out of the maternal side of the placenta and collected with effluent. Stem cells expressing the CD34 cell marker are isolated from the other residual cells and the effluent. The method requires shipment of a whole, intact placenta to the facility for processing so that the cells present within the vasculature of the placenta may be flushed out of the vasculature through the maternal side of the placenta.

Certain methods for isolating, expanding and differentiating fetal stem cells from chorionic villus, amniotic fluid and the placenta and related therapeutic uses are set forth in United States Patent Application Publication No. US 2005/0124003 to Atala et al. Generally, methods for collecting a population of c-kit positive cells are set forth and comprise collecting a piece of chorionic villi or amniotic fluid during pregnancy or, alternatively, a sample of placenta after birth and processing the tissue or fluid to collect c-kit positive cells. The c-kit positive cells sought in the practice of the method express embryonic stage specific cell markers Stage Specific Embryonic Antigen-3 (SSEA-3) and Stage Specific Embryonic Antigen-4 (SSEA-4). In particular, the methods described focus on isolating and culturing a heterogeneous population of cells collected from amniotic fluid, chorionic villi and placenta and selecting the c-kit positive cells by flow cytometry, gradient magnetic selection, and implementation of a solid phase.

Typically, placental cell procurement involves harvesting cells or placental tissue through procedures such as amniocentesis or CVS at a health care facility or, alternatively, through procedures such as transporting whole placentae to a laboratory for processing. In the latter of the two procedures, a laboratory facility generally receives and processes an entire placenta, which requires implementation of resources to collect, ship and process a whole placenta. However, there is no teaching for procuring a suitable size of a piece of human placenta at the bedside post-delivery, and related tissue processing and cell collection methodologies useful for obtaining a yield of viable fetal placental cells expressing CD117.

Accordingly, there is a present need for methods to collect a suitable piece of tissue from a human placenta and, optionally, the umbilical cord, at the bedside, for ready shipment to a processing center for processing to isolate and to store human fetal cells. There is also a present need for methods to process placental tissue to obtain a cell preparation of placental cells derived essentially from the fetal tissue of the placental tissue in accordance with the present invention. There is also a further need to select, culture, isolate and cryopreserve viable fetal placental cells from the population of cells, such as for example, fetal placental cells that express at least one of the cell surface markers CD29, CD44, CD73, CD90, CD105, CD166, SSEA-3 and SSEA-4 and have low or no expression of the cell surface markers CD34, CD45 and CD133. There is yet a further need to cryopreserve a population of fetal placental cells or isolated fetal placental cells obtained from the fetal tissue of the placenta.

### SUMMARY OF THE INVENTION

Methods and processes are provided by the present invention to collect a piece of tissue from a whole human placenta at the bedside after delivery of the placenta for ready packaging and shipment to a processing facility. Methods, processes and systems are also provided by the present invention for processing a piece of placental tissue to isolate, collect, concentrate, and preserve fetal placental stem cells expressing CD117. Optionally, a piece of umbilical cord tissue may be processed in place of the placental tissue. The placental cells are obtained from fetal tissue harvested from a placenta or umbilical cord to produce a population of placental cells which may be cryopreserved or alternately further processed according to the methods and processes of the present invention or any other suitable methods or processes. The population of placental cells are capable of producing a yield of viable fetal placental cells.

A method for obtaining a population of cells enriched for fetal placental cells expressing CD117 is provided by the present invention. The method comprises the steps of disaggregating placental tissue and separating the population of cells from disaggregated placental tissue; collecting and concentrating the population of cells; and cryopreserving the population of placental cells at or below about −135° C.

A process for obtaining a population of placental cells enriched for fetal placental cells is provided by the present invention. The process comprises the steps of procuring placental tissue from a whole placenta, the placental tissue comprising maternal tissue and fetal tissue; optionally removing a substantial portion of the maternal tissue from the placental tissue; disaggregating the placental tissue comprising at least fetal tissue; isolating placental cells from the disaggregated placental tissue; and collecting the population of placental cells by concentrating the population of placental cells with at least one step of centrifugation.

A process for collecting placental cells expressing at least one of the cell surface markers selected from the group consisting of CD29, CD44, CD73, CD90, CD105, CD166, SSEA-3 and SSEA-4 and having low or no expression of at least one of the cell surface markers selected from the group consisting of CD34, CD45 and CD133 from placental tissue comprising fetal tissue is provided by the present invention. The process comprises the steps of isolating the placental cells from the placental tissue; collecting and concentrating the placental cells; and cryopreserving the placental cells.

A system for collecting a population of cells enriched for fetal placental cells is provided by the present invention. The system comprises a placental cell isolater, wherein the placental cell isolator disaggregates placental tissue comprising fetal tissue and separates placental cells from the disaggregate placental tissue; a placental cell collector, wherein the placental cell collector collects the placental cells separated from the disaggregate placental tissue; a placental cell concentrator, wherein the placental cell concentrator concentrates placental cells present in a suspension; and a placental cell cryopreserver, wherein the placental cell cryopreserver maintains the collected and concentrated placental cells at a temperature at or below about −135° C.

A process for isolating a population of fetal placental cells expressing CD117 from a population of placental cells is provided by the present invention. The process comprises the steps of culturing a population of placental cells enriched for fetal placental cells; selecting placental cells expressing CD117 from a culture of the population of placental cells; and cryopreserving the placental cells expressing CD117.

A process for isolating a population of fetal placental cells from a population of placental cells is provided by the present invention. The process comprises selecting placental cells expressing CD117 from a culture of the population of placental cells.

A process for isolating a population of fetal placental cells expressing CD117 from a population of placental cells is provided by the present invention. The process comprises selecting placental cells expressing CD117 from a population of placental cells enriched for fetal placental cells; culturing the placental cells expressing CD117 selected from the population of placental cells enriched for fetal placental cells; and selecting placental cells expressing CD117 from a culture of placental cells.

A population of cells enriched for fetal placental cells obtained from a process of the present invention is provided by the present invention. The process comprises culturing a population of cells comprising fetal placental cells, and selecting cells expressing CD117 from a culture of the population of cells.

A population of fetal placental cells obtained from a process is provided by the present invention. The process comprises selecting placental cells expressing CD117 from a culture of a population of placental cells.

A population of cells enriched for fetal placental cells expressing CD117 is provided by the present invention.

A population of cells enriched for fetal placental cells expressing at least one of the cell surface markers selected from the group consisting of CD29, CD44, CD73, CD90, CD105, CD166, SSEA-3 and SSEA-4 and having low or no expression of at least one of the cell surface markers selected from the group consisting of CD34, CD45 and CD133 is provided by the present invention.

A population of cells enriched for fetal placental cells expressing at least one of the cell surface markers selected from the group consisting of CD29, CD44, CD73, CD90, CD105, CD166, SSEA-3 and SSEA-4 and have low or no expression of at least one of the cell surface markers selected from the group consisting of CD34, CD45 and CD133 is provided by the present invention.

A composition comprising a population of cells enriched for fetal placental cells and a preservation agent is provided by the present invention.

A composition comprising at least one fetal placental cell and a preservation agent is provided by the present invention.

A composition comprising at least one fetal placental cell expressing at least one of the cell surface markers selected from the group consisting of CD29, CD44, CD73, CD90, CD105, CD166, SSEA-3 and SSEA-4 and a preservation agent is provided by the present invention.

A composition comprising at least one fetal placental cell expressing at least one of the cell surface markers selected from the group consisting of CD29, CD44, CD73, CD90, CD105, CD166, SSEA-3 and SSEA-4 and having low or no expression of at least one of the cell surface markers selected from the group consisting of CD34, CD45 and CD133 and a preservation agent is provided by the present invention.

At least one fetal placental cell obtained from a process of the present invention is provided by the present invention. The process comprises procuring placental tissue comprising maternal tissue and fetal tissue of a whole placenta; optionally removing a substantial portion of the maternal tissue from the placental tissue; disaggregating the placental tissue; isolating placental cells from disaggregated placental tissue; collecting and concentrating placental cells in a population of cells; culturing the population of placental cells comprising fetal placental cells; and selecting placental cells expressing CD117 from a culture of the population of placental cells. The process may comprise the further steps of culturing the placental cells expressing CD117 selected from the population of placental cells comprising fetal placental cells; and selecting placental cells expressing CD117 from a culture of placental cells expressing CD117 comprising a population of fetal placental cells.

A method for shipping placental tissue comprising fetal placental cells is provided by the present invention. The method comprising obtaining at least one piece of placental tissue from a whole placenta; packaging the at least one piece of placental tissue to maintain the placental tissue at about 1° C. to about 15° C. for shipment; and shipping the at least one piece of placental tissue to a processing facility, wherein the at least one piece of placental tissue arriving at the processing facility within 72 hours of delivery of the whole placenta.

### BRIEF DESCRIPTION OF THE DRAWINGS

FIG. 1 is an illustration of a view of the fetal side of a whole human placenta having a portion of the proximal umbilical cord attached and showing a scalpel cutting a piece of placental tissue in accordance with the invention.

FIG. 2 is an illustration of a sectional view of a whole human placenta as illustrated in FIG. 1.

FIG. 3 represents a piece of placenta shown in FIG. 1 procured with a scalpel.

FIG. 4 is a flow chart showing generally the overall process of the present invention.

FIG. 5 is a flow chart showing a process of the present invention generally illustrated in FIG. 4 where placental tissue is obtained by punch biopsy, placental tissue is disaggregated by enzymatic digestion of the placental tissue, placental cells are isolated from the placental tissue through cell separation by centrifugation, and the placental cells are collected and concentrated through centrifugation steps and, optionally, cryopreserved.

FIG. 6 is a flow chart showing another embodiment of the process generally illustrated in FIG. 4 where placental tissue is obtained by punch biopsy, placental tissue is disaggregated by enzymatic digestion of the placental tissue, placental cells are isolated from the placental tissue through cell separation by centrifugation, and placental cells are collected with a density gradient in a buffy coat layer that is collected and concentrated through centrifugation steps and, optionally, cryopreserved.

FIG. 7 is a flow chart showing an additional embodiment of the process generally illustrated in FIG. 4 where placental tissue is obtained by punch biopsy, placental tissue is disaggregated by mechanical separation of the placental tissue, the placental cells are isolated from the placental tissue through cell separation with a filter and wash, and the placental cells are collected and concentrated through centrifugation steps and, optionally, cryopreserved.

FIG. 8 is a flow chart showing a further embodiment of the process generally illustrated in FIG. 4 where placental tissue is obtained by punch biopsy, placental tissue is disaggregated by mechanical separation of the placental tissue, placental cells are isolated from the placental tissue through cell separation with a filter and wash, and placental cells are collected with a density gradient in a buffy coat layer that is collected and concentrated through centrifugation steps and, optionally, cryopreserved.

FIG. 9 is a flow chart showing yet another embodiment of the process generally illustrated in FIG. 4 where placental tissue is obtained with scalpel and forceps, placental tissue is disaggregated by enzymatic digestion of the placental tissue, placental cells are isolated from the placental tissue through cell separation by centrifugation, and the placental cells are collected and concentrated through centrifugation steps and, optionally, cryopreserved.

FIG. 10 is a flow chart showing yet an additional embodiment of the process generally illustrated in FIG. 4 where placental tissue is obtained with scalpel and forceps, placental tissue is disaggregated by enzymatic digestion of the placental tissue, placental cells are isolated from the placental tissue through cell separation by centrifugation, and placental cells are collected with a density gradient in a buffy coat layer that is collected and concentrated through centrifugation steps and, optionally, cryopreserved.

FIG. 11 is a flow chart showing yet a further embodiment of the process generally illustrated in FIG. 4 where placental tissue is obtained with scalpel and forceps, placental tissue is disaggregated by mechanical separation of the placental tissue, placental cells are isolated from the placental tissue through cell separation with a filter and wash, and the placental cells are collected and concentrated through centrifugation steps and, optionally, cryopreserved.

FIG. 12 is a flow chart showing another alternative embodiment of the process generally illustrated in FIG. 4 where placental tissue is obtained with scalpel and forceps, placental tissue is disaggregated by mechanical separation of the placental tissue, placental cells are isolated from the placental tissue through cell separation with a filter and wash, and placental cells are collected with a density gradient in a buffy coat layer that is collected and concentrated through centrifugation steps and, optionally, cryopreserved.

FIG. 13a is a flow chart showing an embodiment of the present invention comprising selecting CD117 cells from a cell culture grown from a population of cells collected in accordance with any of the methodologies illustrated in FIGS. 4 through 12 or any other methodologies and then, optionally, cryopreserving the selected CD117 placental cells.

FIG. 13b is a flow chart showing an embodiment of the present invention comprising selecting CD117 placental cells from a population of cells collected in accordance with any of the methodologies illustrated in FIGS. 4 through 12 or any other methodologies and then, optionally, cryopreserving the selected CD117 placental cells.

FIG. 13c is a flow chart showing an embodiment of the present invention comprising selecting CD117 placental cells from a population of cells collected in accordance with any of the methodologies illustrated in FIGS. 4 through 12 or any other methodologies, culturing the placental cells, selecting CD117 placental cells from culture and then, optionally, cryopreserving the selected CD117 placental cells.

FIG. 13d is a flow chart showing an embodiment of the present invention comprising thawing a cryopreserved population of cells collected in accordance with any of the methodologies illustrated in FIGS. 4 through 12 or any other methodologies, culturing the cells, selecting CD117 placental cells from the culture and then, optionally, cryopreserving the selected CD117 placental cells.

FIG. 13e is a flow chart showing an embodiment of the present invention comprising thawing cryopreserved population of cells collected in accordance with any of the methodologies illustrated in FIGS. 4 through 12 or any other methodologies, selecting CD117 placental cells from the culture and then, optionally, cryopreserving the selected CD117 placental cells.

FIG. 13f is a flow chart showing an embodiment of the present invention comprising thawing cryopreserved population of cells collected in accordance with any of the methodologies illustrated in FIGS. 4 through 12 or any other methodologies, selecting CD117 placental cells, culturing the selected CD117 placental cells, selecting CD117 placental cells from the culture and then, optionally, cryopreserving the selected CD117 placental cells.

FIG. 13g is a flow chart showing an embodiment of the present invention comprising thawing a cryopreserved population of cells collected in accordance with any of the methodologies illustrated in FIGS. 4 through 12 or any other methodologies, culturing the cells, selecting CD117 placental cells from the culture, culturing the selected CD117 cells, and then, optionally, cryopreserving the cultured CD117 placental cells.

FIGS. 14a through 14j show representative flow cytometry results for the cells of Cell Line 1005R P3, analyzed for cellular expression of CD44, CD45 and CD117 along with cell viability with 7AAD according to the methods of the present invention. The cells of Cell Line 1005R P3 were obtained by enzymatically digesting a piece of placental tissue comprising maternal placental tissue, collecting and concentrating placental cells as a population of placental cells. The concentrated cells were then cultured through multiple passages that developed into Cell Line 1005R P3. FIGS. 14a through 14j show flow cytometry results at passage 4 of the cell culture. FIGS. 14f through 14j show flow cytometry results at passage 6 of cell culture. The cells of Cell Line 1005R P3 expressed a high percentage of CD44 and CD117 with a high percentage of viability and low or no percentage of CD45 expression.

FIGS. 15a through 15j show representative flow cytometry results for the cells of Cell Line PLF05, analyzed for cellular expression of CD44, CD45 and CD117 along with cell viability with 7AAD according to the methods of the present invention. The cells of Cell Line PLF05 were obtained by enzymatically digesting a piece of placental tissue comprising maternal placental tissue, collecting and concentrating placental cells as a population of placental cells, and cryopreserving the population of cells. The cryopreserved population of cells were thawed and concentrated by centrifugation. The concentrated cells were then cultured through multiple passages according to the methods of the present invention that developed into Cell Line PLF05. FIGS. 15a through 15e show flow cytometry results at passage 6 of the cell culture. The cells of Cell Line PLF05 expressed a high percentage of CD44 and CD117 with a high percentage of viability and low or no percentage of CD45 expression. At passage 6, cells were immunoselected for CD117 according to the methods of the present invention. The cells obtained through the immunoselection in the positive fraction were separately cultured according to the methods of the present invention. FIGS. 15f through 15j show flow cytometry results for the positive fraction of immunoselected cells at passage 6 of the cell culture. The cells of Cell Line PLF05 expressed a high percentage of CD44 and CD117 with a high percentage of viability and low or no percentage of CD45 expression.

FIGS. 16a through 16u show representative flow cytometry results for the cells of Cell Line PLF10, analyzed for cellular expression of CD44, CD45 and CD117 along with cell viability with 7AAD according to the methods of the present invention. The cells of Cell Line PLF10 were obtained by enzymatically digesting a piece of placental tissue comprising maternal placental tissue, collecting and concentrating placental cells as a population of placental cells, and cryopreserving the population of cells. The cryopreserved population of cells were thawed and concentrated by centrifugation. The concentrated cells were then cultured through multiple passages that developed into Cell Line PLF10. FIGS. 16a through 16e show flow cytometry results at passage 8 of the cell culture, and FIGS. 16f through 16j show flow cytometry results at passage 12 of cell culture. The cells of Cell Line PLF10 expressed a high percentage of CD44 and CD117 with a high percentage of viability and low or no percentage of CD45 expression. At passage 4 of the cell culture, cells were immunoselected for CD117 using the methods of the present invention. The cells obtained through the immunoselection in the positive fraction were separately cultured according to the methods of the present invention. FIGS. 16k through 16o show flow cytometry results at passage 7 of the culture of the cells positively selected for CD117. FIGS. 16p through 16t show flow cytometry results at passage 10 of the culture of the cells positively selected for CD117. FIGS. 16u through 16x show flow cytometry results at passage 23 of the culture of the cells positively selected for CD117. The cells of Cell Line PLF10 positively selected for CD117 expressed a high percentage of CD44 and CD117 with a high percentage of viability and low or no percentage of CD45 expression.

FIGS. 17a through 17l illustrate genotyping of cells of Cell Line 1005R P3 performed by Human Identification-Multiplex Short Tandem Repeat (STR) Analysis and the reference samples of maternal blood and cord blood collected at delivery of the placenta. FIGS. 17a through 17d show genotyping analysis of the cells of Cell Line 1005R P3. FIGS. 17e through 17h show genotyping analysis of the cells of the cord blood associated with the placenta processed according to the invention for Cell Line 1005R P3. FIGS. 17i through 17l show genotyping analysis of the cells of the maternal blood collected at delivery of the placenta associated with Cell Line 1005R P3. The STR Analysis involved the performance of PCR analysis on 15 different STR loci plus amelogenin on the X and Y chromosomes. The 15 STR loci analyzed were D8S1179, D21S11, D7S820, CSF1PO, D3S1358, TH01, D13S317, D16S539, D2S1338, D19S433, vWA, TPOX, D18S51, D5S818, and FGA. The amplified product was electrophoresed on ABI 3100 Genetic Analyzer and analyzed using the GeneMapper ID software. Four separate fluorescent dye labels were used to label the samples. The dyes were coupled to PCR primers. Each of the fluorescent dyes emitted its maximum fluorescence at a different wavelength that was detected by the Genetic Analyzer. The analysis was performed by visual inspection of all 15 loci plus amelogenin (X and Y marker). The results show that the cells of the 1005R P3 Cell Line are of 100% fetal origin. In addition, the specimen is of single individual origin. This was established by looking at the STR data for each locus. Each locus (designated by the gray bar at the top of each graph) for a normal individual should have one or two STR alleles. 1005R P3 had alleles 13 and 14 present at D8S1179 locus.

FIGS. 18a through 18l illustrate genotyping of cells of Cell Line PLF10 performed by Human Identification-Multiplex Short Tandem Repeat (STR) Analysis and the reference samples of maternal blood and cord blood collected at delivery of the placenta. FIGS. 18a through 18d show genotyping analysis of the cells of Cell Line PLF10. FIGS. 18e through 18h show genotyping analysis of the cells of the cord blood associated with the placenta processed according to the invention for Cell Line PLF10. FIGS. 18i through 18l show genotyping analysis of the cells of the maternal blood collected at delivery of the placenta associated with Cell Line PLF10. The STR Analysis involved the performance of PCR analysis on 15 different STR loci plus amelogenin on the X and Y chromosomes. The 15 STR loci analyzed were D8S1179, D21S11, D7S820, CSF1PO, D3S1358, TH01, D13S317, D16S539, D2S1338, D19S433, vWA, TPOX, D18S51, D5S818, and FGA. The amplified product was electrophoresed on ABI 3100 Genetic Analyzer and analyzed using the GeneMapper ID software. Four separate fluorescent dye labels were used to label the samples. The dyes were coupled to PCR primers. Each of the fluorescent dyes emitted its maximum fluorescence at a different wavelength that was detected by the Genetic Analyzer. The analysis was performed by visual inspection of all 15 loci plus amelogenin (X and Y marker). The results show that the cells of the PLF10 Cell Line are of 100% fetal origin. In addition, the specimen is of single individual origin. This was established by looking at the STR data for each locus. Each locus (designated by the gray bar at the top of each graph) for a normal individual should have one or two STR alleles. PLF10 has alleles 13 and 14 present at D8S1179 locus.

### DESCRIPTION OF THE PREFERRED EMBODIMENTS

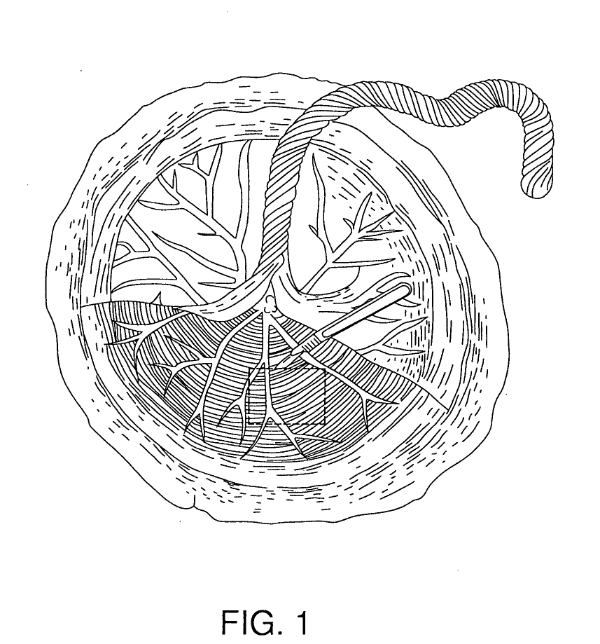

In reference to FIGS. 1 through 18l, the present invention provides methods, processes, and systems for the procurement and processing of placental tissue obtained from a whole human placenta and isolating, collecting and preserving a population of placental cells obtained from the placental tissue. It is recognized that certain placental cells, such as for example, fetal placental cells are pluripotent in nature due to their ability to differentiate into a variety of cells. The term “pluripotent” is used in reference to a cell with complete differentiation versatility, i.e., the capacity to differentiate into at least osteogenic phenotype, hematopoietic phenotype, adipogenic phenotype, myogenic phenotype, hepatic phenotype and endothelial phenotype under suitable conditions for induction. A pluripotent cell possesses the capacity to undergo differentiation into any of the approximately 260 different mammalian cell types. In reference to the present invention, the phrase “cell” is used generally, and the phrase “placental cell” is used specifically to refer to any cell obtained from placental tissue and includes fetal placental cells as defined herein. Additionally, the phrase “fetal placental cell” is used to refer to a placental cell of fetal origin that expresses at least one of the cell surface markers selected from the group consisting of CD29, CD44, CD73, CD90, CD105, CD166, SSEA-3 and SSEA-4 and has low or no expression of the cell surface markers selected from the group consisting of CD34, CD45 and CD133 as obtained and selected by the methods, processes and systems of the present invention.

Fetal placental cells express the antigenic factor CD117 that is also known as a c-kit receptor, Steel factor receptor, and stem cell factor receptor. The gene for c-kit encodes a tyrosine kinase growth factor receptor for Stem Cell Factor (SCF), which is also known as mast cell growth factor and is essential for hematopoiesis, melanogenesis, and fertility. It is recognized that CD117 is expressed in hematopoietic stem cells, mast cells, germ cells, melanocytes, certain basal epithelial cells, luminal epithelium of the breast, and the interstitial cells of Cajal of the gastrointestinal tract. CD117 imparts a critical role in germ cell establishment, maintenance, and function. Research indicates that in the embryonic gonad, CD117 and its corresponding ligand SCF, are essential for the primordial germ cell survival and proliferation. Additionally, research indicates that CD117 and its corresponding ligand SCF are essential for the gamete production in response to gonadotropic hormones. In other words, CD117 in combination with the ligand SCF are necessary for the survival and proliferation of germ cells of the testis, the spermatogonia, and for the growth and maturation of oocytes. Research also indicates that CD117 is a potent growth factor for primitive hematopoietic cell proliferation in vitro.

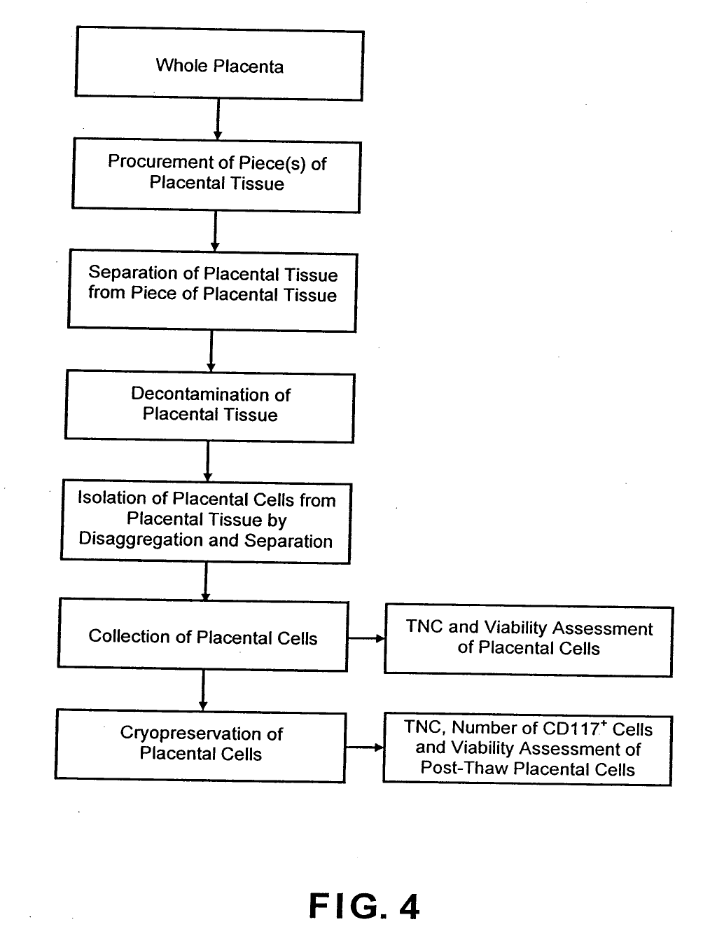

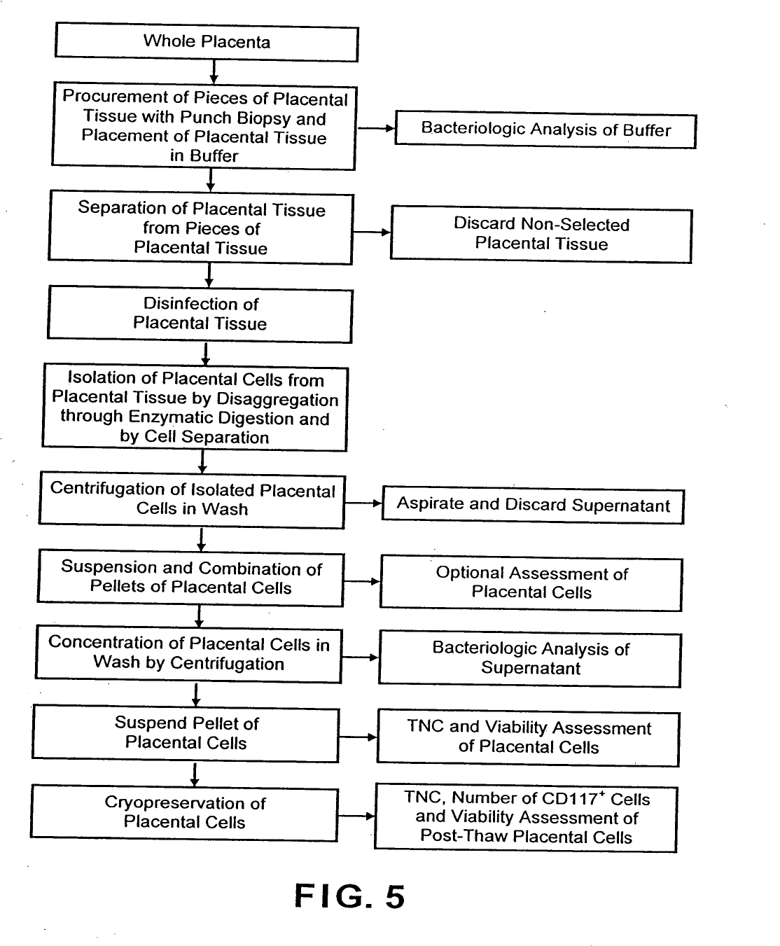

Generally, and as illustrated in FIG. 4, the overall methods, processes and systems of the present invention involve procurement of at least one piece of placental tissue from a whole placenta, separation of fetal placental tissue from the at least one piece of placental tissue; decontamination of the fetal placental tissue; isolation of placental cells including fetal placental cells from the fetal placental tissue by disaggregation and separation; and collection and cryopreservation of the placental cells including fetal placental cells. Pre-processing samples and post-processing samples of collected placental cells may be analyzed with flow cytometry. Bacteriological analysis of the environment of the placental tissue and placental cells may be performed to identify any contamination of the piece of placental tissue and collected placental cells. The overall methods and processes of the present invention are further detailed in various embodiments of the present invention as illustrated and exemplified in FIGS. 5 through 12.

An embodiment of the present invention illustrated in FIG. 5 generally involves obtaining placental tissue by punch biopsy and disaggregating the placental tissue by enzymatic digestion of the placental tissue to release placental cells. After the enzymatic reaction is inhibited, the placental cells are isolated from the placental tissue by centrifugation in a wash. The placental cells including fetal placental cells are then collected and concentrated through several centrifugation steps and, optionally, cryopreserved in the vapor of liquid Nitrogen. Such cryopreservation may occur at or below a temperature of about −135° C.

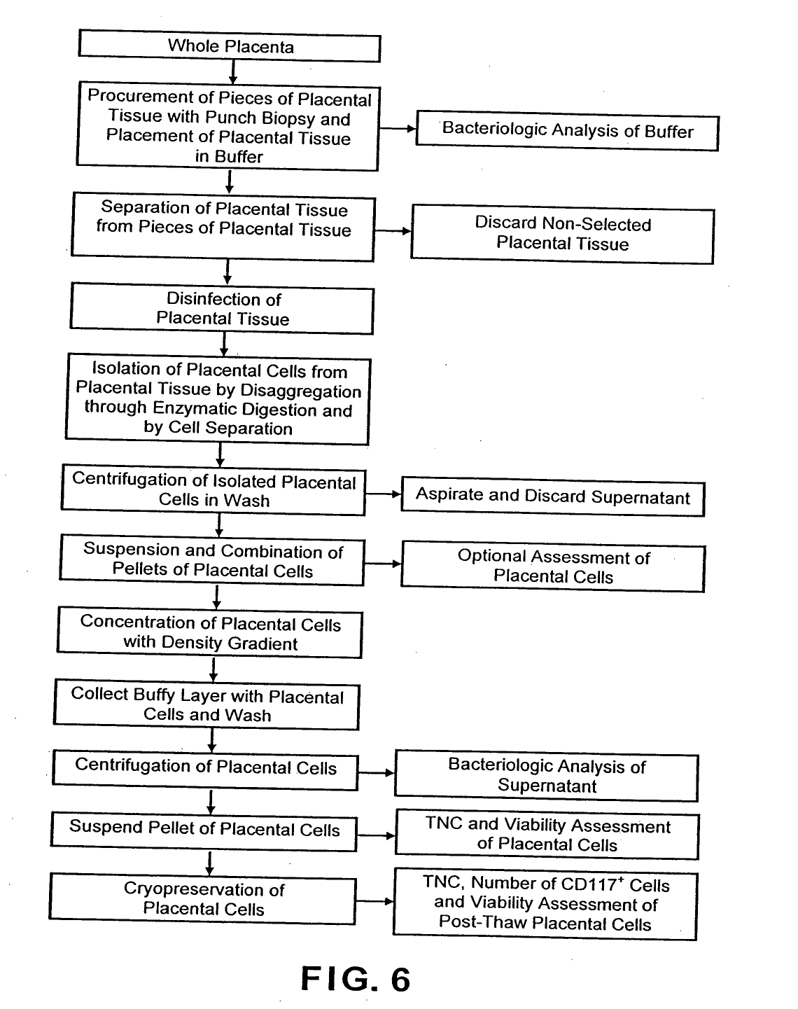

Another embodiment of the present invention illustrated in FIG. 6 generally involves obtaining placental tissue by punch biopsy and disaggregating the placental tissue by enzymatic digestion of the placental tissue to release placental cells including fetal placental cells. After the enzymatic reaction is inhibited, the placental cells including fetal placental cells are isolated from the placental tissue by centrifugation in a wash. The placental cells including fetal placental cells are then collected with a density gradient in a buffy coat layer that is collected and concentrated through several centrifugation steps and, optionally, cryopreserved in the vapor of liquid Nitrogen. Such cryopreservation may occur at or below a temperature of about −135° C.

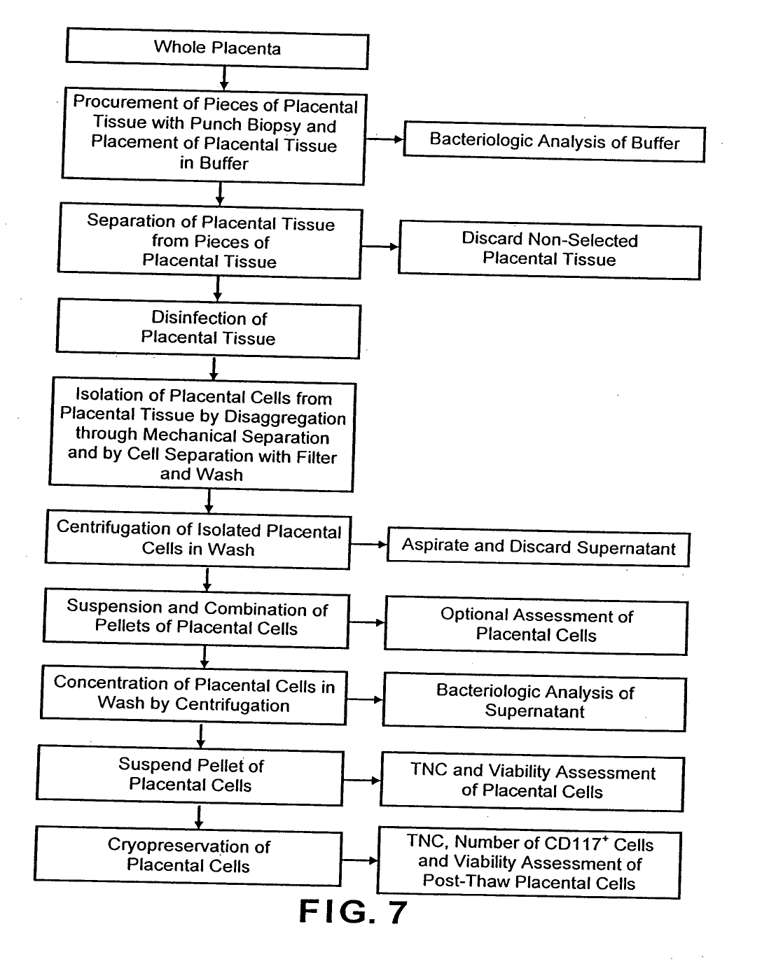

An additional embodiment of the present invention illustrated in FIG. 7 generally involves obtaining placental tissue by punch biopsy and disaggregating the placental tissue by mechanical separation of the placental tissue to release placental cells including fetal placental cells. The placental cells are isolated from the placental tissue through cell separation with a filter and wash. The placental cells including fetal placental cells are then collected and concentrated through several centrifugation steps and, optionally, cryopreserved in the vapor of liquid Nitrogen. Such cryopreservation may occur at or below a temperature of about −135° C.

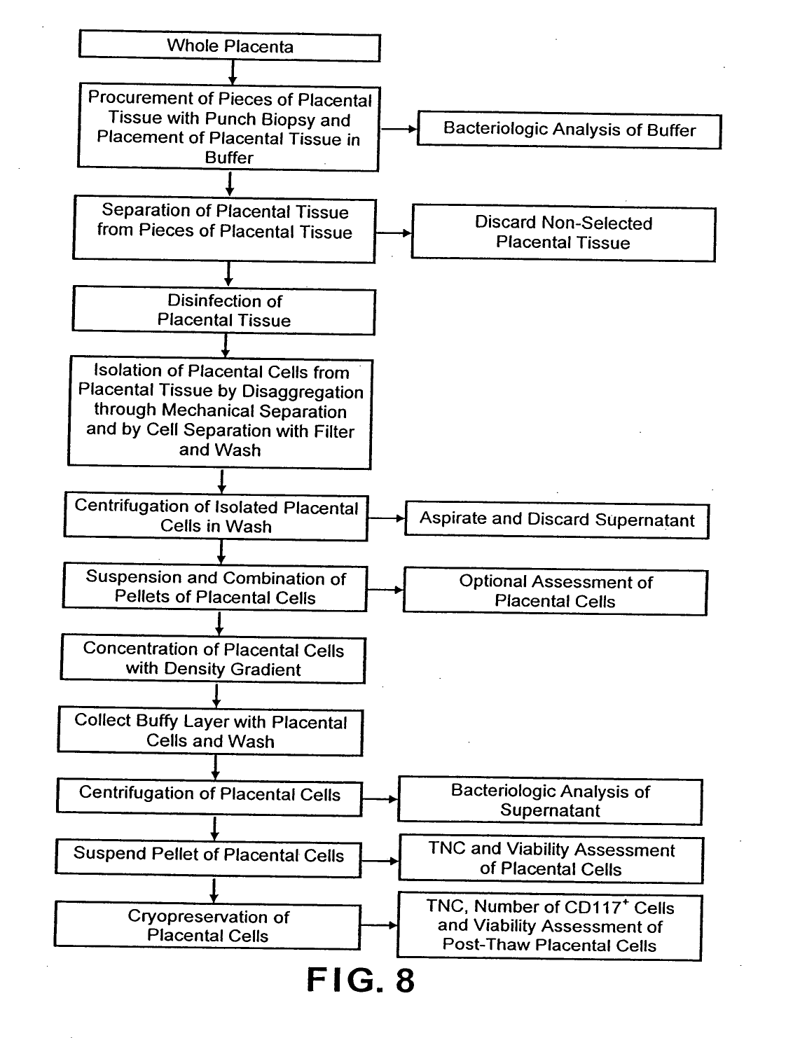

A further embodiment of the present invention illustrated in FIG. 8 generally involves obtaining placental tissue by punch biopsy and disaggregating the placental tissue by mechanical separation of the placental tissue to release placental cells including fetal placental cells. The placental cells are isolated from the placental tissue through cell separation with a filter and wash. The placental cells are then collected with a density gradient in a buffy coat layer that is collected and concentrated through centrifugation, steps and, optionally, cryopreserved in the vapor of liquid Nitrogen. Such cryopreservation may occur at or below a temperature of about −135° C.

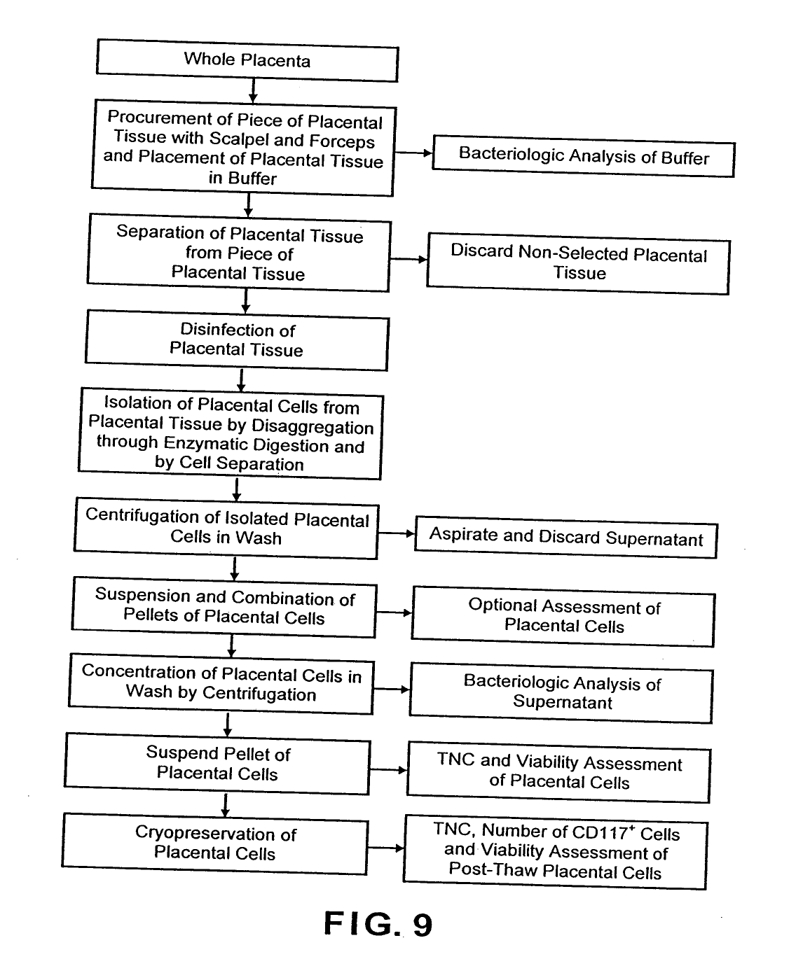

Yet another embodiment of the present invention illustrated in FIG. 9 generally involves obtaining placental tissue with scalpel and forceps and disaggregating placental tissue by enzymatic digestion of the placental tissue to release placental cells including fetal placental cells. After the enzymatic reaction is inhibited, the placental cells are isolated from the placental tissue by centrifugation in a wash. The placental cells are then collected and concentrated through centrifugation steps and, optionally, cryopreserved in the vapor of liquid Nitrogen. Such cryopreservation may occur at or below a temperature of about −135° C.

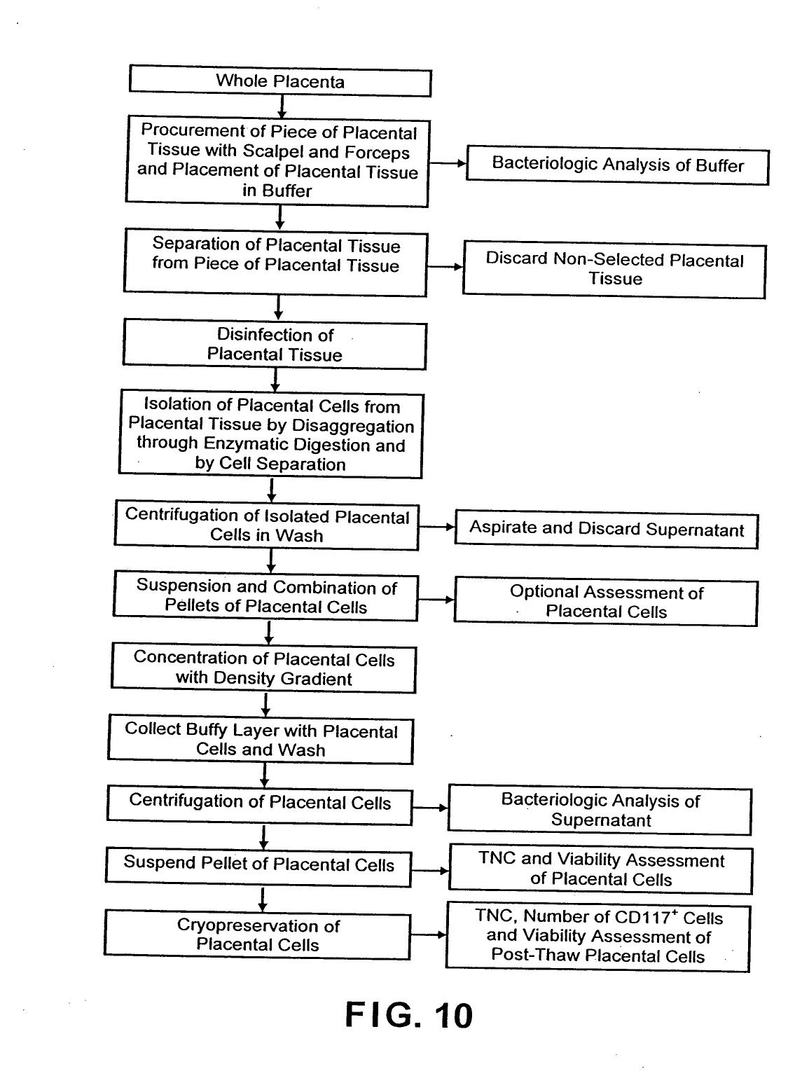

Yet an additional embodiment of the present invention illustrated in FIG. 10 generally involves obtaining placental tissue with scalpel and forceps and disaggregating the placental tissue by enzymatic digestion of the placental tissue to release placental cells including fetal placental cells. After the enzymatic reaction is inhibited, the placental cells are isolated from the placental tissue by centrifugation in a wash. The placental cells are then collected with a density gradient in a buffy coat layer that is collected and concentrated through centrifugation steps and, optionally, cryopreserved in the vapor of liquid Nitrogen. Such cryopreservation may occur at or below a temperature of about −135° C.

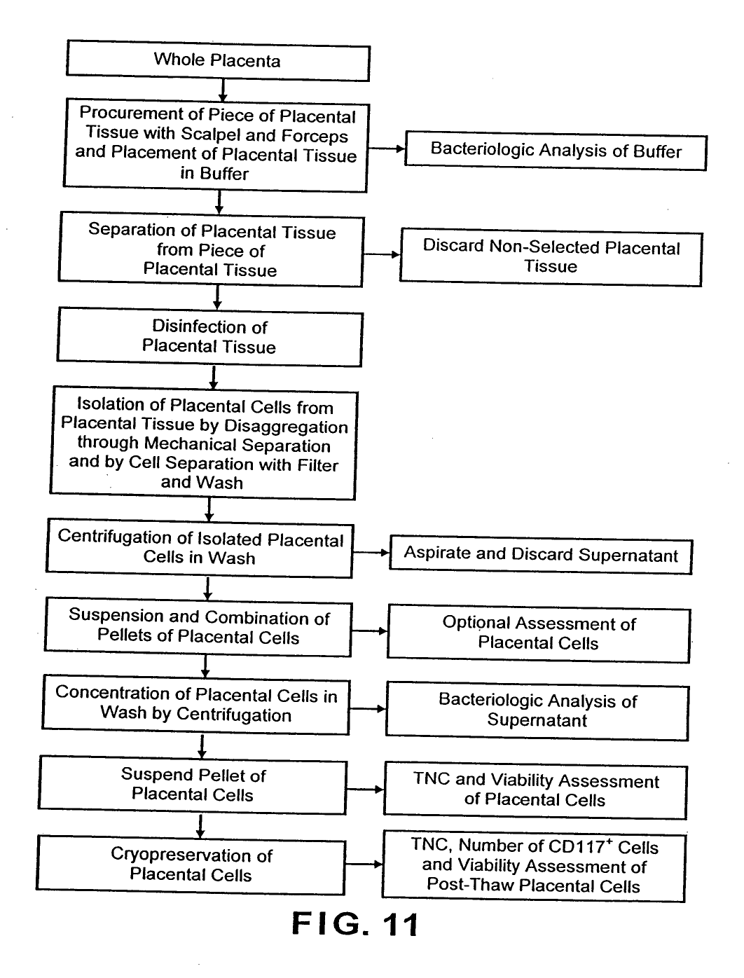

Yet a further embodiment of the present invention illustrated in FIG. 11 generally involves obtaining placental tissue with scalpel and forceps and disaggregating placental tissue by mechanical separation of the placental tissue to release placental cells including fetal placental cells. The placental cells are isolated from the placental tissue through cell separation with a filter and wash. The placental cells are then collected and concentrated through centrifugation steps and, optionally, cryopreserved in the vapor of liquid Nitrogen. Such cryopreservation may occur at or below a temperature of about −135° C.

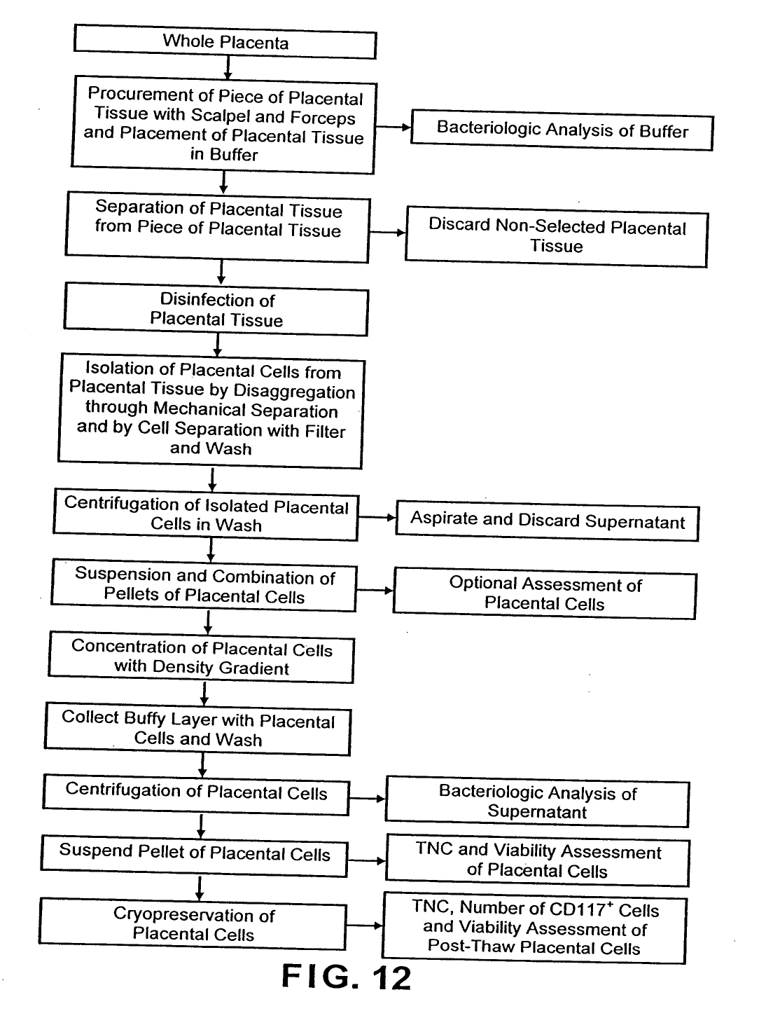

Another alternative embodiment of the present invention illustrated in FIG. 12 generally involves obtaining placental tissue with scalpel and forceps and disaggregating placental tissue by mechanical separation of the placental tissue to release placental cells including fetal placental cells. The placental cells are isolated from the placental tissue through cell separation with a filter and wash. The placental cells are then collected with a density gradient in a buffy coat layer that is collected and concentrated through centrifugation steps, and optionally cryopreserved in the vapor of liquid Nitrogen. Such cryopreservation may occur at or below a temperature of about −135° C.

The aforementioned embodiments of the present invention as referenced in FIGS. 4 through 12 are described in more detail as provided hereinafter under the following headings Collecting, Labeling and Transporting Placental Tissue; Initial Processing of Placental Tissue at Processing Facility; Microbiological Quality Control at Pre-Processing of Sample; Disinfection of Placental Tissue; Preparation for Disaggregation of Placental Tissue; Disaggregation by Mechanical Separation; Disaggregation by Enzymatic Digestion; Centrifugation of Placental Cells; Concentration of Placental Cells by Centrifugation; Concentration of Placental Cells with Density Gradient for Cryopreservation; Preparation for Cryopreservation; Cryopreservation; and Flow Cytometry Analysis.

Collecting, Labeling and Transporting Placental Tissue

The placenta is collected and treated after vaginal or caesarean delivery in preparation for shipment to a processing facility. A maternal blood sample is collected and subjected to analysis to identify any blood borne infectious disease as surrogate (mother) testing for the donor cells (baby) and other analysis, such as, for example genotyping. Optionally, a sample of cord blood may also be collected and subjected to analysis. A placenta tissue collection kit is provided to collect, treat and package a piece of placenta for shipment. The placental tissue collection kit comprises a placental tissue transport container such as a box, bag or other container suitable for shipping a piece of placenta preferably at a cold temperature; a sterile tissue container of suitable size including, but not limited to, a 500 ml container; Dulbecco's Phosphate Buffer Saline (DPBS) (Mediatech or suitable replacement) that contains no calcium, magnesium or phenol red; plastic zipped bags with absorbent towels; sterile scalpel; sterile forceps; sterile ruler; an optional tincture of Iodine or alternatively Povidone-iodine or Betadine; and sterile 4×4 gauze; sterile basin, and sterile gloves. Alternatively, in place of the sterile scalpel, a sterile punch biopsy (for example, a punch biopsy of 2 mm, 4 mm, 8 mm or other suitable size) is provided for performing punch biopsies in the placental tissue collection kit. The placental tissue collection kit is taken to the hospital or birthing center by the donor where the placental tissue collection is completed according to instructions in the kit. Appropriate barrier and personal protection measures including sterile gloves are used throughout the handling of the placenta and procurement of placental tissue.

Prior to collecting the placental tissue, the donor's identity is confirmed and assigned an accession number and specimen labels are to be implemented to identify the placental tissue in the sterile tissue container. Each specimen label must be completed with date, time, and donor information including name and identity number such as social security number or other assigned information number, an accession number, and initials of individual procuring the placental tissue.

After the placenta is delivered, the whole placenta is placed in a sterile basin. The sterile container is opened and the DPBS buffer is aseptically poured into the sterile container in preparation to receive the placental tissue obtained from the whole placenta.

An area of the fetal surface of the placental disc is prepared to obtain a piece of placental tissue. The area of the fetal surface may be wiped lightly with the tincture of Iodine by placing the tincture of Iodine in the center of area to be cleaned. The tincture of Iodine is circled outwards to cover the whole area of the fetal surface to be cleaned. The Iodine remains on the placenta for about 30 seconds before the Iodine is wiped dry with the sterile gauze. Preferably, a tincture of Iodine may be used. Alternatively, Povidone-iodine or Betadine may also be used in place of the tincture of Iodine. A further alternative may be to prepare the area of the fetal surface of the placenta by wiping the area with sterile gauze. Yet a further alternative may be to collect the piece of placenta as is, as delivered.

A piece of placental tissue may be procured from the prepared area of the placenta optionally disinfected by using the sterile scalpel and forceps of the placental tissue collection kit as shown in FIG. 1. Optionally, sterile scissors may be used in place of the sterile scalpel to procure a piece of placental tissue.

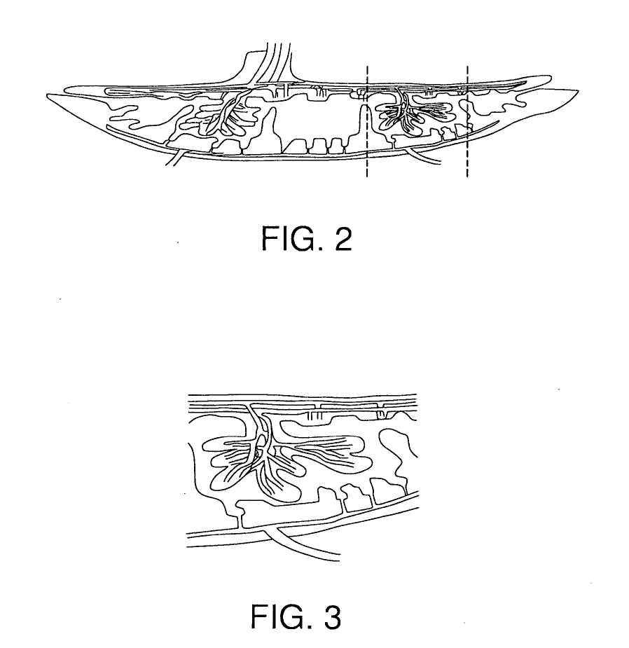

In reference to FIGS. 1 through 3, a piece of tissue of the placenta is cut away from the area of the placenta, optionally disinfected with the tincture of Iodine or alternative, using the sterile ruler, scalpel, and forceps of the placental tissue collection kit. The sterile ruler is used to measure on the fetal side of the placenta, i.e., the cord side of placenta, the size of the piece of placental tissue to be cut. In another embodiment, two pieces of placental tissue may be procured. Once measured, a piece of placental tissue is aseptically cut using the sterile scalpel and sterile forceps as shown particularly in FIGS. 1 and 2. The size of the width and height of the piece of placental tissue may be between about 4 cm by about 10 cm to about 8 cm by about 13 cm, preferably between about 6 cm by about 12 cm to about 7 cm by about 12 cm. The width and height of the piece of placenta are measured on the fetal surface side of the placenta as represented in FIG. 1. The piece of placental tissue may include a maternal portion and a fetal portion of the placenta and may also include chorionic villi as shown in FIG. 3.

Alternatively, several punch biopsies of tissue of the placenta are procured from the area of the placenta, optionally disinfected with the tincture of Iodine or alternative, using the punch biopsy, which may be provided in the placental tissue collection kit.

Many punch biopsies are obtained from the area of the placenta. Punch biopsies are removed aseptically from the placenta using the sterile punch biopsy and sterile forceps. At least ten to twenty or other suitable number of punch biopsies of placental tissue are removed from the placenta to obtain placental tissue. The punch biopsy will be used repeatedly to yield the desired amount of placental tissue including chorionic villi.

Once procured from the placenta, the piece of placental tissue as shown in FIG. 3 or punch biopsies of the placental are submerged in the DPBS media in the sterilized container using the sterile forceps provided with the placental tissue collection kit. The sterilized container is tightly closed with the corresponding lid and packaged for transportation. The tissue of the placenta is to remain cold from procurement through shipment and processing at the processing facility. It is preferred that the piece of placental tissue will remain at a temperature between about 1° C. to about 15° C., and preferably at a temperature between about 1° C. to about 10° C. for the duration of shipment to the processing facility.

The sterilized container is packaged for transportation by placing the sterilized container in a large plastic zip bag that is sealed by closing the zip structure of the bag. The large plastic bag with sterilized container enclosed is placed in a second large zip bag and is also sealed. Four absorbent towels are placed in the bottom of the placental tissue transport container. Two double bags full of wet ice are prepared. One of the double bags full of wet ice is placed on top of the absorbent towels. The sterilized container in the sealed bags is placed on top of the ice bag. The second double bag full of wet ice is placed on top of the specimen, and the remaining absorbent towels are placed on top of the second bag of ice. The placental tissue transport container is closed, secured and sealed. Other suitable containers may be used for transportation of the sterilized container so long as the container maintains the piece of placental tissue at a temperature between about 1° C. to about 15° C. and, preferably, between about 1° C. to about 10° C. during shipment. For example, other suitable containers include but are not limited to cooled and insulated shipping containers sold by Therapak Corporation. A preprinted label must be provided on the placental tissue transport container next to the air bill. A carrier may be contacted within at least two (2) hours of birth to pick up shipment of the placental tissue transport container. The carrier may be AirNet or other preferred courier suitable for transporting biological materials. The placental tissue shipment container should arrive at the processing facility within about 24 hours to about 72 hours of collection. It is preferred that the placental tissue shipment container arrive at the processing facility between about 24 hours to about 48 hours after collection and, particularly, within about 48 hours after collection.

For multiple births, each sample of placental tissue should be separately procured in accordance with the invention. Each placental tissue sample should be placed in its own labeled sterile container, and each sample should then be shipped with its corresponding paperwork to the processing facility in a separate placental tissue shipment container. The placental tissue shipment container may be left at room temperature prior to pick up by the courier. However, at no time should the placental tissue shipment container be stored in a hot environment. All sharps devices are removed prior to packaging the sterilized container and shipping the placental tissue shipment container.

Initial Processing of Placental Tissue at Processing Facility

The placental tissue shipment container is received at the processing facility and all donor information is obtained and inputted into the processing facility records. Once the donor information is confirmed, all tubes, batch records and blood culture bottles for microbiological detection are affixed with appropriate labels prior to processing. Appropriate barrier and personal protection measures are used throughout handling the placental tissue and blood at the processing facility. Once received at the processing facility, the sterilized container is removed from the placental tissue shipment container, placed on ice in an ice pan, and transferred into a clean room in a biological safety cabinet (BSC) for placental tissue disinfection and disaggregation and fetal placental cell isolation, collection and optional preparation for cryopreservation.

Microbiological Quality Control at Pre-Processing of Sample

Aseptic techniques are used throughout the microbiological quality control at pre-processing of the sample and particularly in the BSC. In the BSC and using a sterile technique, the top of the sterilized container is removed. A labeled blood culture bottle should be visually inspected to ensure that the culture bottle is suitable for use. The visual inspection should be used to identify signs that would preclude use of the culture bottle such as turbidity, signs of gas production and/or evidence of growth (i.e. yellow sensor). Only blood culture bottles without signs of turbidity, gas production and/or evidence of growth should be used. The plastic flip top may be removed without touching the septum of the blood culture bottle. A sterile alcohol pad may be used to disinfect the septum. A new sterile alcohol pad may be opened and placed on top of the septum by touching the edges of the alcohol pad only. Using a syringe, a volume of DPBS media surrounding the procured tissue in the sterilized container is removed and used to inoculate the labeled blood culture bottle. Alternatively, two culture bottles may be inoculated with a sample for individual aerobic and anaerobic detection of bacteria. The volume of DPBS media may be between about 1.0 to about 4.0 ml. The blood culture bottle is incubated at about 37° C. in the automated microbiological detection system, preferably BacT/ALERT by Biomerieux or other suitable collection system so long as it is validated according to the organisms it will detect and used according to manufacturer's specifications for blood culture. The BacT/ALERT blood culture bottle and system are provided as an example and not a limitation of a suitable collection system for the practice of the invention. Other suitable automated or manual blood culture specimen bottles and systems may be used as long as it is in compliance with 21 C.F.R. Section 610.12.

Disinfection of Placental Tissue

Aseptic techniques are used throughout disinfection of the placental tissue and particularly in the BSC. The placental tissue is disinfected to rid the placental tissue of contaminants. For example, the placental tissue may be subjected to a double treatment and wash for disinfection. The double treatment comprises a Betadine solution and an antibiotic dip solution. The Betadine solution may be prepared as about 50% Betadine (10% Povidone-iodine Topical wash solution—Purdue Products or other suitable product) and about 50% PBS buffer or alternatively HBSS or other suitable solution as an initial disinfecting wash. The antibiotic dip solution may be a triple antibiotic solution, which is dependent upon tissue contaminants. For example and not a limitation, the antibiotic dip solution may be prepared by dissolving 1 g/vial of desiccated Cefazolin with about 10 ml HBSS, 2×1 g/vial Streptomycin with about 10 ml HBSS per vial, 2×50 mg/vial of desiccated Amphotericin B with about 10 ml HBSS per vial. Once solubilized, the antibiotic dip solutions are removed from each vial and all of the antibiotic solutions are combined with HBSS up to a final volume of about 150 ml. The double treatment is prepared by filling about 150 ml of the Betadine solution in a first sterile disposable container in the BSC, about 150 ml of the prepared antibiotic dip solution in a second sterile disposable container in the BSC, and about 150 ml of DPBS in a third sterile disposable container in the BSC.

The double treatment is carried out in a BSC where the piece of placental tissue or, alternatively, the punch biopsies in the DPBS are removed from the sterile container and placed in a sterile Petri dish present in the BSC.

Placental tissue processing may comprise selecting fetal tissue or fetal and maternal tissue of placenta. A varied degree of thickness of the placental tissue may be used. By way of example, the entire piece of placental tissue comprising maternal tissue and fetal tissue may be processed. Certain fibrous tissue and blood vessels may be removed. Alternatively, if fetal placental tissue is to be selected, the tissue of the fetal side of the placenta, including chorionic villi, may be separated from the tissue of the maternal side of the placenta in a BSC using sterile scissors, scalpel, and forceps. The maternal tissue would be discarded as biological waste. A further alternative may be to obtain a piece of placental tissue with a sufficient depth to obtain a suitable amount of fetal cells.

The selected placental tissue is disinfected with the double treatment. The placental tissue is first washed in about 50 ml of DPBS. The placental tissue is then dipped and fully submerged in the Betadine solution in a first disposable container for about 5 seconds and then removed. The placental tissue is dipped and fully submerged in the antibiotic dip in a second disposable container for between about one minute to about 3 minutes and then removed. The placental tissue is finally dipped and rinsed in the DPBS wash in a third disposable container for between about 5 seconds to about 10 seconds and then removed. After disinfection, the placental tissue is placed in a sterile container filled with DPBS so that the placental tissue is submerged in the DPBS. The sterile container is placed on ice in an ice pan for further disaggregation processing in accordance with the invention.

Preparation for Disaggregation of Placental Tissue

Aseptic techniques are used throughout isolation of placental cells from the selected placental tissue in the BSC. Throughout the process of isolating placental tissue, gloves should be wiped frequently with about 70% IPA. Gloves that are visibly contaminated with blood or reagents at anytime during the process should be discarded and replaced with new gloves. Any spills or drips should be immediately cleaned with Cavicide or another suitable EPA registered disinfectant. All materials should be wiped with about 70% IPA or Cavicide prior to placement under the BSC. The BSC should be disinfected with about 70% IPA or Cavicide before and after processing.

The manufacturer, lot number and expiration dates of all media, reagents, enzymes or solutions used in the practice of the invention are documented on the batch record. Red biohazard bags are in biohazard trash bins and sharps containers are prepared to receive discarded biological waste products and spent sharps devices. A vacuum collection system for aspirating material is assembled by attaching one end of the vacuum tubing to a pump and the other end to the “vac” port of a collection flask. Attach one end of the remaining vacuum tubing to the flask at the “patient” port. Squeeze the other end of the tubing into one of the metal slots at the side of the BSC.

A sterile stainless steel pan should be placed in the BSC for sterile supplies after the BSC has been disinfected. All sterile supplies used for the process should be placed in the pan. The sterile supplies include sterile forceps, scalpels, scissors, needles, syringes and Petri dishes. Other materials should be placed in the BSC including red top tubes, rack for red top tubes, blood culture bottles, alcohol pads, 50 ml conical collection tubes, rack for 50 ml conical collection tubes, pipettes, and ice pan. All media, enzymes and reagents are preferably placed on ice and are stored between about 2° C. to about 8° C.

Media and reagents are prepared for the isolation process. A wash solution may be used throughout the placental cell isolation process. For example, the wash solution comprises about 500 ml of HBSS with or without Calcium chloride and Magnesium chloride (Gibco), about 5 ml of Heparin (Heparin Sodium 1,000 USP Units/ml—American Pharmaceutical Partners), about 2.5 ml of DNase (Genentech—at a concentration of 2.5 mg/ml) and about 50 ml of protein (Human Serum Albumin (HSA) 25% or other suitable concentrations—Telacris Biotherapeutics). The wash solution is placed in a container that is on ice in an ice pan in the BSC.

Before the tissue is disinfected, the placental tissue may be placed in a Petri dish with DPBS, where the excess maternal decidual tissue may be scraped from the amniochorion tissue. The tissue may be washed two to three times to remove all decidual tissue before the tissue is decontaminated. Any blood vessels or fibrous tissue including blood may be scraped from the seamless piece of amniochorion tissue. The cleaned amniochorion tissue is decontaminated via the antibiotic dip. The placental tissue is then chopped, cut and minced into small pieces using a sterile scalpel and forceps. The reduction of the placental tissue to small pieces complements the disaggregation of the placental cells from the placental tissue using either mechanical separation or enzymatic digestion.

Disaggregation by Mechanical Separation

In preparation for mechanical separation, sterile 50 ml conical tubes are labeled and placed in a sterile tube holder in the BSC. Sterile cell strainers are placed in the BSC. Prior to use, each cell strainer may be removed from its packaging and may only handled through contact with the handle of the cell strainer while taking special care to avoid contact with the filter of the cell strainer. When moving the cell strainer, gloves must be confirmed to be sterile or have immediately been cleaned with IPA. A cell strainer may be a BD Falcon Cell Strainer (100 micron Nylon cell strainer) or other suitable strainer. A conical tube is donned with a cell strainer applied as a cover to the tube.

Pieces of minced placental tissue are removed from the Petri dish and placed in the cell strainer and washed with wash solution using a sterile pipette. A sterile syringe may be removed from a package by grasping the luer end to retain sterility of the handle. The handle of the syringe will be used to assist in tissue disaggregation by forcing the handle against the placental tissue in the cell strainer. The force placed against the placental tissue will act to disaggregate the placental tissue and release placental cells, which are forced through the pores of the filter of the cell strainer by force from the handle. Wash solution may be periodically placed on the placental tissue to wash placental cells through the cell strainer. The placental tissue may also be squeezed with forceps while in the cell strainer to release cells into the cell strainer, and the cells are then either forced or washed through the cell strainer. Additional placental tissue may be added to the cell strainer via sterile forceps for processing. These steps may be repeated until all of the minced placental tissue is disaggregated and forced against the cell strainer in order to obtain a maximum yield of placental cells from the placental tissue in the conical collection container. When a conical collection container is filled to about 45 ml, the cell strainer may be moved to the next conical collection container to continue the process until all of the minced placental tissue is disaggregated. When the cell strainer becomes clogged with debris, the cell strainer may be replaced with a new cell strainer. All wash solution in the Petri dish may be removed with a 10 ml pipette and rinsed through the cell strainer to collect cells in the collection container. Each conical collection container is placed on ice after it is filled with wash solution and the cells that have been forced through the cell strainer.

Alternatively, mechanical disaggregation may occur by other suitable manual and automated mechanical disaggregation systems or other suitable system adapted for releasing placental cells from placental tissue. For example, the BD Medimachine™ automated, mechanical disaggregation system may be automated to mechanically disaggregate pieces of placental tissue up to about one cm³ with a volume of wash solution and a filter membrane with about 100 μm pore size in accordance with the manufacturer's instructions for disaggregation. The disaggregated cells are collected in collection tubes and subjected to centrifugation to concentrate the placental cells.

Disaggregation by Enzymatic Digestion

An enzyme is prepared for enzymatic digestion of the minced placental tissue. A suitable enzyme is collagenase or other suitable enzyme. For example, collagenase may be reconstituted by adding about 10 ml of cold HBSS to every 500 mg of desiccated collagenase to reconstitute the enzyme and create a final enzyme concentration of about 50 mg/ml. Alternatively, the final enzyme concentration may be 100 mg/ml. The HBSS and collagenase solution may be swirled gently for mixing. The solution should not be shaken vigorously to avoid degradation of the enzyme. The creation of bubbles during reconstitution of collagenase should be avoided. If the enzyme is already prepared and frozen, obtain one vial about 30 minutes prior to use and allow it to thaw in an ice bath protected from light exposure.

The collagenase solution may be combined with HBSS to create a digestion solution. The digestion solution comprises a mixture of collagenase solution and HBSS so that the collegenase is at a concentration capable of digesting the connective tissue of the placental tissue. For example, a suitable concentration of collegenase is about 100 mg/ml of digestion solution. The digestion solution can be prepared by reconstituting every about 1 g of collegenase with about 10 ml of cold HBSS and using about 25 ml of HBSS and about 0.035 ml of 2.5 mM CaCl. The digestion solution at a volume of about 10 ml may be placed in each labeled 50 ml conical collection tube. DNAse is added to the digestion solution in each conical collection tube, preferably at a volume between about 5 drops to about 10 drops per tube. DNase may be added using a sterile pipette where the DNAse is at a concentration of about 2.5 mg/ml. A volume of about 0.25 ml of preservative free Heparin (Heparin Sodium 1,000 USP Units/ml—American Pharmaceutical Partners) is added to each 25 ml of the digestion solution in each conical collection tube to prevent clotting if placental blood is still present. The final concentration of the Heparin in the digestion solution is about 10 μg/ml. A sufficient amount of conical collection tubes are labeled and filled with digestion solution depending upon the amount of placental tissue that will be enzymatically digested and collected.

The small pieces of placental tissue should be of a suitable size so the enzyme will be able to break down the collagen in the connective tissue of the placental tissue to free the placental cells. The placental tissue may be chopped into approximately about 1 cm³ sizes. However, the placental tissue may be chopped into pieces that are smaller than about 1 cm³ and even larger than about 1 cm³. Up to about 6 grams of placental tissue may be placed into each conical collection tube containing about 10 ml of digestion solution with DNAse and Heparin.

The conical collection tubes containing the digestion solution and placental tissue may be placed in a 37° C. incubator for an appropriate amount of time to digest the connective tissue of the placental tissue to release the placental cells. For example, the conical collection tubes may be incubated for about 30 minutes for enzymatic digestion. The tubes may be inverted a few times at intervals during the incubation period. If it appears that the placental tissue has not undergone sufficient digestion, the enzymatic digestion may continue in the incubator for additional time up to about one hour at about 37° C.

Once a sufficient amount of enzymatic digestion has occurred, the enzymatic reaction may be stopped by adding an appropriate amount of protein to inactivate the enzyme. For example, the enzymatic digestion may be stopped by adding 25% Human Serum Albumin to the digestion solution and digested placental tissue in the conical collection tube. About 5 ml of HSA may be added to each tube to neutralize the enzyme. The conical collection tube may be inverted several times to mix the HSA with the digestion solution. Once well mixed, the conical collection tubes are centrifuged to wash out the enzyme. The conical collection tubes are centrifuged at about 2000 rpm for about 7 minutes within a range of about 2° C. to about 30° C. After the conical collection tube is removed from the centrifuge, the supernatant above the pellet may be aspirated with suction while avoiding the pelleted cells. The pelleted cells in the bottom of the conical collection tube are re-suspended in a solution such as DPBS, DMEM or other suitable solution. Once re-suspended, the cells may be optionally filtered through a cell strainer and rinsed with wash solution into 50 ml conical collection tubes. Each conical collection tube is placed on ice.

Alternatively, the enzyme digestion may occur in sequential steps. For example and not as a limitation, about up to 6 grams of placental tissue may be added into a first digestion solution of about 10 ml in a first tube. The first tube is incubated, optionally in a sterile bag, at about 37° C. in an incubator for about 10 minutes. The placental tissue is removed from the digestion solution in the first tube and placed in a second tube with about 10 ml of new digestion solution. The digestion solution in the first tube may be discarded. The second tube with digestion solution and placental tissue is incubated, optionally, in a sterile bag, at about 37° C. in an incubator for about 20 minutes. The tube may be inverted at about 10 minutes. The placental tissue is removed from the second tube and placed in a third tube containing about 10 ml of new digestion solution. The enzymatic reaction in the enzyme digestion solution in the second tube may be stopped by adding about 4 ml 25% HSA (Baxter with concentrations of 1000 units per ml) and mixing the digestion solution with the HSA. The second tube is then placed on ice.

The third tube containing about 10 ml of digestion solution and placental tissue is incubated, optionally in a sterile bag, at 37° C. in an incubator for about 20 minutes. The tube is inverted at about 10 minutes. The placental tissue is removed from the third tube and placed in a fourth tube containing 10 ml of digestion solution. The enzymatic reaction in the third tube is stopped by adding about 4 ml 25% HSA (Baxter with concentrations of 1000 units per ml) and mixing the digestion solution with the HSA. The third tube is then placed on ice.

The fourth tube containing about 10 ml of digestion solution and placental tissue is incubated, optionally in a sterile bag, at 37° C. in an incubator for about 20 minutes. The tube is inverted at about 10 minutes. The placental tissue is removed from the fourth tube and placed in a fifth tube containing about 10 ml of digestion solution. The enzymatic reaction in the fourth tube is stopped by adding about 4 ml 25% HSA (Baxter with concentrations of 1000 units per ml) and mixing the digestion solution with the HSA. The fourth tube is then placed on ice.

The fifth tube containing about 10 ml of digestion solution and placental tissue is incubated, optionally in a sterile bag, at 37° C. in an incubator for about 20 minutes. The tube is inverted at about 10 minutes. The placental tissue is removed from the fifth tube and discarded. The enzymatic reaction in the fifth tube is stopped by adding about 4 ml 25% HSA (Baxter with concentrations of 1000 units per ml) and mixing the digestion solution with the HSA. The fifth tube is then placed on ice.

The digestion solution containing placental cells in the second through fifth tubes is filtered through a filter, for example and not a limitation, a 100 micron filter. About 10 ml of wash solution per tube may be used to wash and rinse the second through fifth tubes into the filter and to wash and rinse the filter. The filtered digestion solution and wash solution may be collected in 50 ml collection tubes. The 50 ml collection tubes are centrifuged at about 2000 rpm for about 7 minutes at about 20° C. The supernatant is removed from the collection tubes and the pelleted cells are re-suspended in about 45 ml of wash solution. The re-suspended cells are filtered through a 100 micron filter and into a 50 ml collection tube. The filter is rinsed with wash solution. The filtered, re-suspended cells and wash solution are combined into a 50 ml collection tube and wash solution is added to bring the total volume up to about 35 ml. The collection tube is centrifuged at about 2000 rpm for about 7 minutes at about 20° C.

The supernatant is subject to microbiology quality control. The pellet comprising placental cells is re-suspended with wash solution up to about 5 ml. The suspension of placental cells may be analyzed by flow cytometry and a total nucleated cell count may be performed.

Explant Methodology

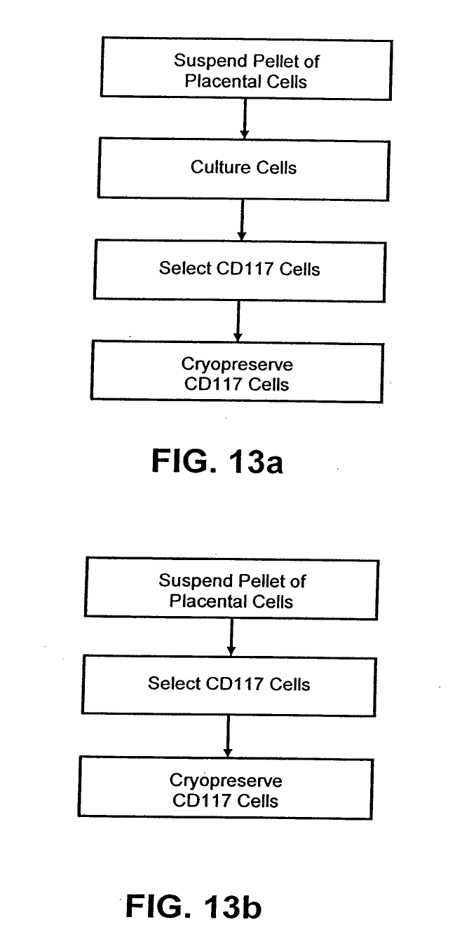

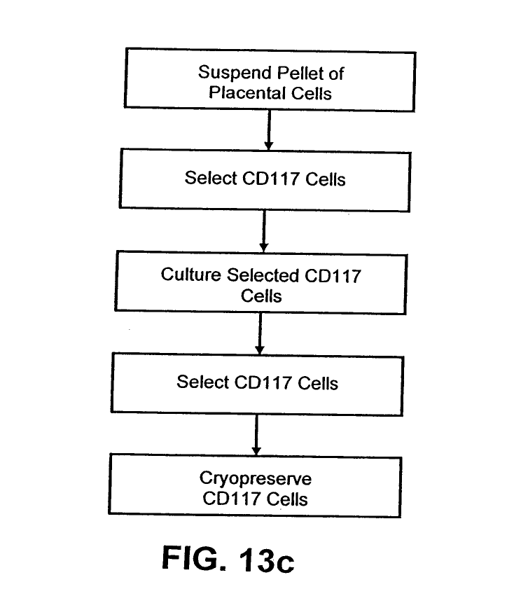

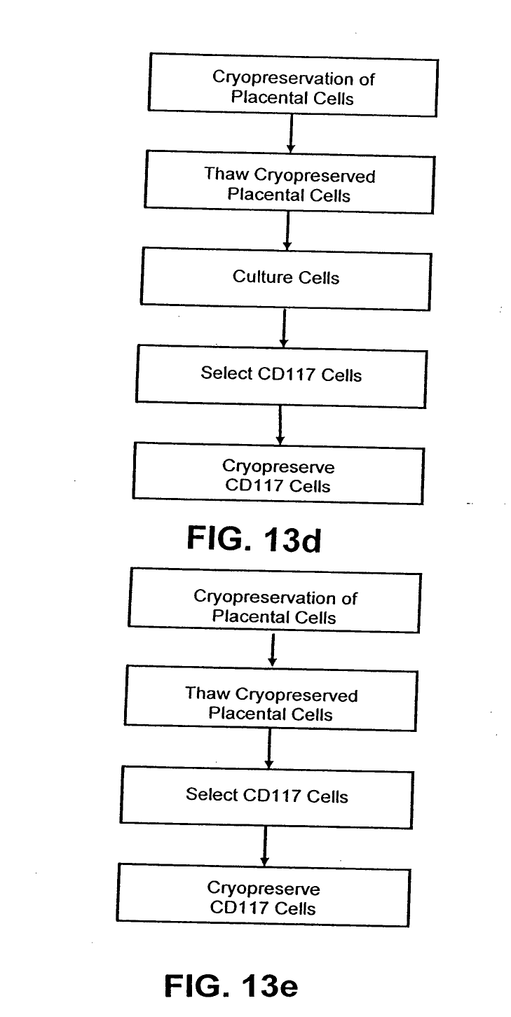

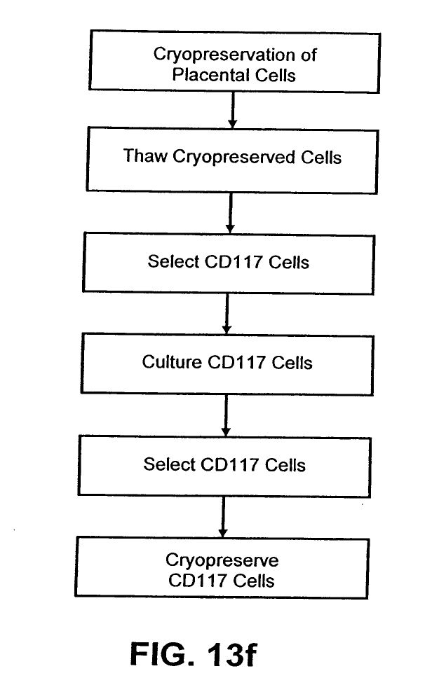

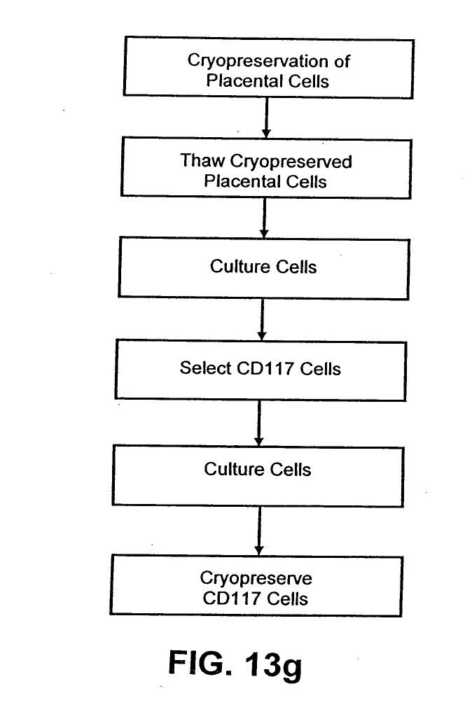

As an alternative to disaggregation by mechanical separation or enzymatic digestion, explant methodologies may be used to collect placental cells from the placental tissue. For example and rather than mincing the placental tissue, the placental tissue may be cut with a sterile scalpel or scissors along the fetal portion of the placenta to obtain one or several pieces of fetal placental tissue about 2 mm². The squares of placental tissue would be placed in an untreated tissue culture flask and allowed to dry and adhere to the flask. The time for allowing the squares of tissue to dry and adhere may be about 30 minutes to about 60 minutes. Then, tissue culture complete media, such as for example Chang's complete media would be added to the flask containing the adhered squares of placental tissue. The flask would be incubated for about 7 days to about 28 days at about 37° C. at 5% CO₂ in an incubator at which time the placental cells would leave the pieces of square placental tissue and adhere to the flask. The cells should become visible around the square pieces of placental tissue. Cell culture passages would be routinely performed according to the cell culture passage methods of the present invention. The cell cultures may be dissociated from the flask using Trypsin and according to the methods described herein. The cells collected from the cell culture may be concentrated according to the centrifugation methods of the present invention. The cells collected from the cell culture may also be cryopreserved, cultured and/or immunoselected according to the methods of the present invention, such as for example, by the methods described in FIGS. 13a through 13g, or any other suitable methods.

Centrifugation of Placental Cells

The placental cells in the wash solution collected in conical collection tubes from either mechanical separation or enzymatic digestion may be subjected to centrifugation to concentrate the placental cells. The wash solution containing placental cells are equally separated into conical tubes for centrifugation. Centrifugation comprises subjecting the placental cells suspended in the wash solution to about 2000 rpm for about 7 minutes between about 2° C. to about 30° C. The conical collection tubes are removed from the centrifuge and the supernatant is aspirated with suction and discarded. A suitable volume of wash solution is used to re-suspend the pelleted cells in each tube. For example, about 2 ml to about 3 ml of wash solution may be used to re-suspend the pelleted cells. All of the re-suspended placental cells in the wash solution are transferred into a 50 ml conical collection tube, and the volume of the cellular suspension in the conical collection tube may be brought up to a total volume of about 31 ml with the wash solution. About one ml of the cellular suspension is removed and placed in a pre-processing collection tube for testing the total nucleated cell count and viability if to be assessed with Trypan blue or other suitable viability testing system. The about 30 ml of cellular suspension may be, optionally, filtered through a cell strainer to remove any residual debris such as disaggregated connective tissue or unwanted cells such as red blood cells from the cellular suspension. The cellular suspension may be subjected to centrifugation and/or density gradient to concentrate the placental cells in the cellular suspension obtained by disaggregation by either mechanical separation or enzymatic digestion.

Concentration of Placental Cells by Centrifugation

The cellular suspension may be concentrated through centrifugation. The suspension may be centrifuged in the conical collection tube at about 2000 rpm for about 7 minutes at between about 2° C. to about 30° C.

Bacteriological analysis of the supernatant is performed using the BacT/ALERT system or other suitable system for microbial analysis. The plastic flip top is removed from the culture bottle without touching the septum of the culture bottle, which is disinfected with a sterile alcohol pad. A new sterile alcohol pad is opened and placed on top of the septum of the culture bottle. A sterile syringe used to collect between about one ml to about 4 ml of supernatant from the conical collection tube after centrifugation and to inoculate the BacT/ALERT blood culture bottle with the supernatant. Alternatively, two culture bottles may be inoculated with a sample of supernatant for individual aerobic and anaerobic detection of bacteria. The BacT/System is incubated at about 37° C. in a BacT/ALERT system in accordance with manufacturer's instructions. The remaining supernatant is aspirated with suction while carefully avoiding the pelleted cells at the bottom of the conical collection tube.

The pelleted cells are re-suspended with wash solution up to about 6 ml. About one ml of cellular suspension may be removed and placed into a post-processing tube and may be tested for the total nucleated cell count, cell viability and flow cytometric analysis for CD117 and other cell surface markers. About 5 ml of suspension is further processed in preparation for cryopreservation. Alternatively, a 10, 20 or 30 ml suspension may be cryopreserved from each piece of tissue.

Concentration of Placental Cells with Density Gradient for Cryopreservation

The cellular suspension may be concentrated using a density gradient. A density gradient of about 15 ml is placed at the bottom of the conical tube containing the about 30 ml of cellular suspension so that the cell suspension is above the density gradient. For example, the density gradient may be Lymphocyte Separation Medium (Density 1.077-1.083 g/ml at about 20° C.—Cellgro) or other suitable gradient which may have a higher or lower density. The conical collection tube containing the cellular suspension and the density gradient is subject to centrifugation. For example, the conical collection tube may be centrifuged at about 1,400 rpm for about 30 minutes between about 15° C. to about 30° C., preferably at about 20° C., and without a brake applied to slow the centrifuge.

As a result of density gradient centrifugation, a buffy coat layer forms at the interface between the supernatant and the density gradient. The buffy coat layer contains the desired cellular population obtained from the placental tissue. The supernatant above the buffy coat layer is aspirated to about 5 ml above the buffy coat layer and discarded. The buffy coat layer may be removed by gently swirling the buffy coat layer and reaming the sides of the conical tube at the buffy coat layer with a 10 ml pipette. The smallest volume of density gradient as possible should be collected with the buffy coat layer. The remaining density gradient and pellet containing red blood cells is discarded. The density gradient separation may be performed with reagents and cells at room temperature.

The buffy coat layer is placed in a 50 ml conical collection tube and the volume is brought up to about 30 ml with wash solution. The cellular suspension is subject to centrifugation at about 2000 rpm for about 7 minutes between about 15° C. to about 30° C.

Bacteriological analysis of the supernatant is performed using the BacT/ALERT blood culture bottle. The plastic flip top is removed from the culture bottle without touching the septum, which is disinfected with a sterile alcohol pad. A new sterile alcohol pad is opened and placed on top of the septum of the culture bottle. A sterile syringe is used to collect one ml of supernatant from the conical collection tube after centrifugation and to inoculate the BacT/ALERT blood culture bottle with the supernatant. Alternatively, two culture bottles may be inoculated with sample for individual aerobic and anaerobic detection of bacteria. The BacT/ALERT blood culture bottle is incubated at about 37° C. in a BacT/ALERT system according to manufacturer's specifications.

The remaining supernatant is aspirated with suction while carefully avoiding the pelleted placental cells. The pellet may be resuspended with wash solution up to about 6 ml. About one ml of suspension may be removed and placed into a post-processing tube for flow cytometry analysis as described below. The post-processing sample will be tested for the total nucleated cell count. About 5 ml of cellular suspension may be diluted up to a 10 or 20 ml suspension for further processed in preparation for cryopreservation.

Preparation for Cryopreservation

The about 5 ml of cellular suspension obtained by either density gradient concentration or centrifugation is combined with a cryopreservation agent in preparation for cryopreservation. The cryopreservation agent comprises a buffer, a protein, and a preservative. For example, the cryopreservation agent is about 5 ml solution comprising about 3 ml of the buffer DPBS, about one ml of the protein HSA (Telacris Bio), and about one ml of the preservative DMSO (99% Stemsol). Optionally, the DMSO concentration may be used in about 5% to about 10% concentration. Alternatively, sterile filtered autologous plasma from the corresponding cord blood collected from the donor/baby may replace the protein and buffer. Moreover, the cryopreservation agent may comprise DMSO/Dextran 40. The cryopreservation agent may be made by first combining the desired volume of DPBS and HSA and chilling the mixture for about 10 minutes on ice, and then adding about one ml of DMSO and chilling for about 10 minutes on ice. The cryopreservation agent is carefully added to the about 5 ml of cellular suspension to a total volume of about 10 ml of mixture of cellular suspension and cryopreservation agent. The mixture may be separated into desired aliquot volumes in several vials adapted for cryopreservation or maintained in one volume in one tube or may, alternatively, be maintained in a bag designed for cryopreservation. For example, the mixture is separated into 5 ml bar-coded cryovials and five separate one ml QC vials adapted for cryopreservation.

Cryopreservation

The population of placental cells and cryopreservation agent in the cryopreservation vials may be subjected to several temperature reduction steps to reduce the temperature of the population of cells comprising fetal placental cells and/or other cells to a final temperature of about −90° C. utilizing a controlled rate freezer. Suitable control rate freezers include, but are not limited to, Planar Controlled Rate Freezer Kryo 10/16 (TS Scientific), Cryomed Thermo Form a Controlled Rate Freezer 7454 (Thermo Electron, Corp.) The following temperature reduction steps may be programmed in the controlled rate freezer: first reducing the mixture of the population of cells and cryopreservation agent to about 4° C. and then reducing the mixture at about 1° C. per minute to about −3° C., and then about 10° C. per minute to about −20° C., and then about 1° C. per minute to about −40° C., and finally about 10° C. per minute to about −90° C. Alternatively, a program may be used that reduces the temperature of the mixture by approximately 1 to 2° C. per minute. The cryovials containing the mixture of the population of cells and cryopreservation agent are placed in the controlled rate freezer and subjected to the temperature reduction steps. Once the mixture and cryopreservation agent reaches about −90° C., the cryopreservation vials are transferred to a cryogenic storage unit and stored in the vapor of liquid Nitrogen at a temperature at or below about −135° C. For example, a suitable cryogenic storage unit includes, but is not limit to, LN2 Freezer MVE 1830 (Chart Industries).

Flow Cytometry Analysis

The pre-processing sample, post-processing samples and any other samples containing cells may be collected and tested for total nucleated cell count, cell viability by Trypan blue via dye exclusion and any cell surface markers, for example and not a limitation CD117.

The total nucleated cell count may be quantified by an automated hematology analyzer (Sysmex XE-2100), by hand count with a hemocytometer or other means suitable for obtaining cell count.

A flow cytometry analysis of any sample may be performed by spinning the mixture of the population of cells and cryopreservation agent in a vial in a centrifuge at about 2000 RPM for about 7 minutes between about 2° C. to about 30° C. until complete or alternatively between about 15° C. to about 30° C. After centrifugation, the supernatant may be pipetted off and the pellet of cells is diluted in a wash solution. If a population of cells were frozen and need to be thawed, the vials may be agitated in a 37° C. water bath and mixed by inversion while avoiding a complete thaw. Quality control may be performed to assess the total number of nucleated cells (TNC), the number of cells expressing the cell surface marker CD117 or any other cell surface marker, and cell viability using 7AAD. Cells expressing the cell surface marker CD117 may be assessed by flow cytometry using a monoclonal antibody against CD117 with a fluorescent label. For example, suitable monoclonal antibodies include, but are not limited to, BD Pharmingen PE antihuman CD117 (YB5.B8) and CD117-PE (104D2D1) or CD117-PE (95C3) both from Beckman Coulter. The analysis of cells for expression of other cell surface markers may be assessed by flow cytometry using polyclonal or monoclonal antibodies for a specific cell surface marker that may be expressed on a cell. Further TNC cell recovery may be calculated in accordance with the invention.

For flow cytometry analysis, accurately pipette approximately between about 0.5 million cells to about 10.0 million cells of a well-mixed sample of the population of cells into two tubes and vortex briefly. Verify that sample and tube accession numbers match.

Wash the cells in the two tubes with about one ml of wash solution and centrifuge in Blood Bank Serofuge for about one minute. Decant supernatant without disturbing the pellet. Add about 100 uL of wash solution with a micropipetter back into each tube. Vortex briefly. In the tube for testing, add from about 5 ul to about 10 uL of CD117-PE dependent upon assay validation, about 20 uL of CD44-FITC, about 10 uL of CD45-ECD, and about 20 uL of 7-AAD Viability dye. In the tube for isotype control, add about 5 uL to about 10 uL of IgG-PE dependent upon assay validation, about 20 uL of IgG-FITC, about 10 uL of IgG-EDC and about 20 uL of 7AAD Viability dye. Incubate the tubes at room temperature (between about 15° C. to about 30° C.) for about 20 minutes while protecting from light exposure. If sample contains red blood cells with a hematocrit of greater than 5%, lyse for about 10 minutes and protect from light. However, if sample was collected by density gradient or thawed, do not lyse sample. Program carousel work list on FC500 flow cytometry instrument or other suitable flow cytometry instrument. If sample was not lysed, wash after about 20 minutes incubation with about one ml of wash solution and decant supernatant. If sample was lysed, serofuge sample and decant supernatant. Add wash solution up to about one ml and spin again and decant supernatant. Add about one ml of Sheath fluid, vortex and run on FC500 or other suitable flow cytometer.

CD117 positive cell count and cell viability may be reported from the flow cytometry reports and transcribed to work documents for samples. The sample includes the isotype control result which may be subtracted, if applicable, from a total CD117 count and documented on the CD117 report. The instrument used to run post sample may be recorded on the work document. Results may be assessed for discrepancies between values determined and discrepant samples may be repeated before reporting.

Calculations

The total nucleated cell count (TNC) may be converted as TNC (×10³/uL) to TNC (×10⁶/ml). Pre-count TNC equals TNC (×10⁶/ml)×Volume (ml).

Data Collection

All collected processing information may be recorded in the batch record for each sample of placental tissue and may be bound by day in a folder including batch record, cell count data worksheets, CD117 viability worksheets and Freezerworks or other suitable inventory management and planar record system.

Current Good Manufacturing Practice (cGMP) standards and current Good Tissue Practice (cGTP) established by the United States Food and Drug Administration in the Code of Federal Regulations are followed throughout the practice of the invention.

The aforementioned steps of the processes, methods and systems of the present invention may be alternatively embodied as illustrated by FIGS. 4 through 12.

### Further Embodiments of the Invention

Another embodiment of the invention illustrated in FIG. 13a generally comprises culturing a population of cells isolated and collected in accordance with the methodologies of the present invention. The culturing of the population of cells may occur after the placental cells are collected as illustrated in FIGS. 4 through 12. After cell culturing, this embodiment comprises selecting CD117 cells from the cell culture and then optionally cryopreserving the selected CD117 cells in accordance with the present invention.

A further embodiment of the invention illustrated in FIG. 13b generally comprises selecting CD117 cells from the population of cells isolated and collected in accordance with the methodologies of the present invention. The selecting of the CD117 cells may occur after the placental cells are collected and suspended as illustrated in FIGS. 4 through 12. After selection, the embodiment comprises optionally cryopreserving the CD117 cells.

A further embodiment of the invention illustrated in FIG. 13c generally comprises selecting and culturing CD117 cells from the population of cells isolated and collected in accordance with the methodologies of the present invention. The selection of the CD117 cells may occur after the placental cells are collected and suspended as illustrated in FIGS. 4 through 12. After selection, the embodiment comprises culturing CD117 cells in accordance with the present invention. After cell culturing, the embodiment comprises selecting CD117 cells from the cell culture and then optionally cryopreserving the selected CD117 cells in accordance with the methodologies of the present invention.

Yet another embodiment of the invention illustrated in FIG. 13d generally comprises culturing a population of cells isolated and collected in accordance with the methodologies of the present invention. The culturing of the cells may occur after cryopreserving placental cells as shown in FIGS. 4 through 12 and then thawing the cryopreserved placental cells. After cell culturing, this embodiment comprises selecting CD117 cells from the cell culture and then optionally cryopreserving the selected CD117 cells in accordance with the present invention.

Yet a further embodiment of the invention illustrated in FIG. 13e generally comprises selecting CD117 cells from the population of cells isolated and collected in accordance with the methodologies of the present invention. The selecting of the CD117 cells may occur after cryopreserving placental cells as shown in FIGS. 4 through 12 and then thawing the cryopreserved placental cells. After selection, the embodiment comprises optionally cryopreserving the CD117 placental cells in accordance with the present invention.

A further embodiment of the invention illustrated in FIG. 13f generally comprises selecting and culturing CD117 cells from the population of cells isolated and collected in accordance with the methodologies of the present invention. The selecting of the CD117 cells may occur after cryopreserving placental cells as shown in FIGS. 4 through 12 and then thawing the cryopreserved placental cells. After selection, the embodiment comprises culturing CD117 placental cells in accordance with the present invention. After cell culturing, the embodiment comprises selecting CD117 cells from the cell culture and then optionally cryopreserving the CD117 cells in accordance with the present invention.

Yet a further additional embodiment of the invention illustrated in FIG. 13g generally comprises thawing a cryopreserved population of cells, if cryopreserved, collected in accordance with the methodologies of the present invention. The thawing may occur after cryopreserving placental cells as shown in FIGS. 4 through 12. After thawing, the embodiment comprises culturing the cells in accordance with the invention and then selecting CD117 placental cells from the culture. The present invention comprises further culturing the selected CD117 cells, and then optionally cryopreserving the cultured CD117 placental cells in accordance with the present invention.

The aforementioned embodiments of the present invention as referenced in FIGS. 13a through 13g are described in more detail as provided hereinafter under the following headings: CD-117 Cell Selection, CD117 Cell Separation, Preparation for Cell Culture, Cell Culture and Cell Lines.

CD-117 Cell Selection

A population of cells obtained in accordance with the methodologies of the present invention comprises fetal placental cells expressing at least the cell surface marker CD117. The present invention includes the further steps of culturing the population of cells and/or selecting placental cells expressing CD117 as shown generally in FIGS. 13a through 13g and described in further detail herein.

The steps of selecting and isolating placental cells expressing CD117 from the population of cells may occur (a) after the population of cells including placental cells expressing CD117 are collected and concentrated through several centrifugation steps and cultured as disclosed herein and referenced in FIG. 13a; (b) after the population of cells including placental cells expressing CD117 are collected and concentrated through several centrifugation steps as disclosed herein and referenced in FIG. 13b; (c) after the population of cells including the placental cells expressing CD117 are cryopreserved and then thawed and cultured as disclosed herein and referenced in FIG. 13d; (d) after the population of cells including the placental cells expressing CD117 are cryopreserved and then thawed as disclosed herein and referenced in FIG. 13e; and (e) at any other suitable time in the practice of the invention when CD117 cells may be selected from a population of cells, such as for example, as shown in FIGS. 13c and 13g. The steps of selecting and isolating placental cells expressing CD117 and, optionally, any other cell expressing CD117 collected in accordance with the methods the present invention provides a population of enriched cells expressing CD117, which may be used for further cell culture or optional cryopreservation in accordance with the methodologies of the present invention.

The step of selecting placental cells expressing CD117 from the population of cells comprises labeling placental cells with anti-human CD117 antibodies and then labeling the CD117 stem cell-anti-human CD117 antibody complexes with magnetically-labeled antibodies capable of binding to the anti-human CD117 antibodies. Additionally, the method comprises labeling any cell expressing CD117 with anti-human CD117 antibodies and then labeling the CD117 cell-anti-human CD117 antibody complexes with magnetically-labeled antibodies capable of binding to the anti-human CD117 antibodies. The method of selecting placental cells expressing CD117 may include selecting any cell expressing CD117 that is collected in accordance with any of the methodologies of the present invention. The step of isolating placental cells comprises exposing the complexes comprising CD117 cells, anti-human CD117 antibodies, and magnetically-labeled antibodies to a magnetic field to draw the magnetically-labeled antibodies and the rest of the complex to the column, and washing all other CD117 negative cells through the column for analysis.

Throughout the steps of selecting and isolating placental cells expressing CD117, the cellular suspension of the population of cells and working buffer may be maintained at a cold temperature. The cellular suspension may comprise a population of cells suspended in a wash solution if the steps of selecting and isolating placental cells expressing CD117 occurs (a) after concentration of placental cells, suspension of the pellet and culturing the cellular suspension as shown in FIG. 13a, or (b) after concentration of placental cells and suspension of the pellet as shown in FIG. 13b. Alternatively, the cellular suspension may comprise a population of cells suspended in cryopreservation agent if the steps of selecting placental cells expressing CD117 occurs (a) before cryopreservation, thawing and culturing the cells as shown in FIG. 13d or 13g, or (b) after cryopreservation and thawing as shown in FIG. 13e.

The cellular suspension may be centrifuged at about 300 g for about 10 minutes. The pellet may be suspended in a working buffer with anti-human CD117 antibodies. For example, the working buffer may comprise, for example and not as a limitation, PBS at about pH 7.2, bovine serum albumin, EDTA and about 0.09% Azide (or suitable replacement) (BD Biosciences). The pellet may be suspended, for example, in about 100 ul of working buffer and about 5 ug of purified antibodies having affinity for human CD117. The antibody may be monoclonal or polyclonal. The antibody may be purified IgG or other antibody capable of binding human CD117. The antibody may be a mouse anti-CD117 antibody. For example, the antibody may be a monoclonal mouse anti-human CD117 antibody (available as 104D2 from Santa Cruz or YB5.58 from BD Biosciences).

The solution comprising the cells, working buffer and anti-CD117 antibodies are incubated for a first incubation period. For example, the first incubation period may comprise between about 20 minutes to about 25 minutes on ice. The incubation period may, alternatively, be shortened to less than about 20 minutes if the temperature is at least about 2° C. to about 8° C. or about 5 to about 10 minutes if at least at room temperature. After the incubation period, the solution with the cells may be washed with working buffer to remove unbound antibody and then centrifuged. For example, the centrifugation may occur at about 300 g for about 10 minutes. After centrifugation, the supernatant is aspirated and may be saved for analysis, and the pellet is suspended in working buffer. For example, the volume of the working buffer may be about 80 ul.

A second batch of antibodies having microbeads affixed thereto and having an affinity for the anti-human CD117 antibody are added to the working buffer used to suspend the pellet. The microbeads may comprise, for example, iron oxide and polysaccharide. The microbeads may be biodegradable. The microbeads are available through Miltenyi Biotec. For example, the second batch of antibodies are specific for an antibody having affinity for human CD117, such as for example, a goat anti-mouse IgG antibody. The antibody may be monoclonal or polyclonal. The antibody may be capable of binding to the light chain and/or the heavy chain of mouse antibodies. The antibody may be, for example, a goat anti-mouse IgG microbead conjugate available through Miltenyi Biotec as product 130-048-401. A 2 ml vial of the aforementioned goat anti-mouse IgG may be used for approximately 1.0×10ˆ9 of total un-separated cells.

The cellular suspension may be incubated for a second incubation period. For example, the incubation period may be in a range of about 30 minutes to about 35 minutes. Alternatively, the incubation period may be less than about 30 minutes where the incubation occurs at about 2° C. to about 8° C. or about 5 to about 10 minutes where incubation occurs at about room temperature. After the incubation period is complete, the cells are washed with working buffer, such as for example, about 2 ml of working buffer, and the cells are then centrifuged. For example, the centrifugation may occur at about 300 g for about 10 minutes. The supernatant may be aspirated and saved for analysis, and the pellet containing cells is suspended in working buffer, such as for example, about 500 ul of working buffer.

CD117 Cell Separation

The placental cells expressing the cell surface marker CD117 may be separated from a cellular suspension in working buffer using a MS column to separate the CD117 placental cells. For example, an MS Column (Miltenyi Biotec) or other suitable column may be used. Alternatively, other suitable methods to separate cells may be used. A MiniMACS kit (Miltenyi Biotec) comprising a unit, multistand, MS columns and microbeads may be used for CD117 cell selection. The MS column may be prepared by rinsing it with working buffer. For example, the volume of working buffer used to rinse the column may be about 500 ul. The column is placed in a magnetic field of a MACS separator (Miltenyi Biotec) or suitable separator providing a magnetic field.

The cellular suspension in working buffer is added to the column with a pipette or other device capable of transferring a volume of liquid. The CD117 placental cells labeled with anti-human CD117 antibodies, which are bound with antibodies attached to microbeads, are held in the column due to the magnetic field of the MACS separator. Any unlabeled cells, along with the working buffer, flow through the column and may be collected in a sterile tube for cell phenotyping and cell count. The unlabeled cells, which flow through the column, may be identified as a negative fraction. The column may be washed with working buffer after adding the cellular suspension. For example, the column may be washed at least three times or any amount of time that causes all or substantially all of the unlabeled cells to pass through the column. The effluent from the washing steps may be collected for cell phenotyping and count. The effluent may also be identified as a negative fraction.

The labeled CD117 placental cells may be collected from the column after the column is washed. The labeled CD117 cells are collected by placing a sterile tube under the column and removing the column from the magnetic field. Once the column is removed from the magnetic field, the labeled CD117 placental cells pass through the column and into the sterile tube. Residual labeled CD117 placental cells in the column may be washed out by adding working buffer to the column to wash the cells through the column and, optionally, by stripping the column with a plunger to release the cells. The collected labeled CD117 placental cells may be identified as the positive fraction. In order to obtain a more purified population of labeled CD117 cells, the positive fraction may, optionally, be run through a column at least one more time following the previously disclosed washing procedure. The positive fraction may be centrifuged at about 300 g for about 10 minutes and the supernatant aspirated. The pellet may be suspended in about 5 ml of working buffer.

The positive fraction and the negative fraction are analyzed with a hemocytometer to obtain a total count of viable cells. The negative fraction is analyzed by flow cytometry for phenotyping. Optionally, the positive fraction may be analyzed by flow cytometry for phenotyping.

The positive fraction containing CD117 placental cells may be prepared for cryopreservation in accordance with the methods of the present invention and described herein in further detail. About one ml of human serum albumin, about 3 ml of DPBS and about one ml of DMSO may be added to the about 5 ml of the positive fraction. Alternatively, other tissue culture media may be used in the step of preparing the CD117 placental cells for cryopreservation, such as for example, complete media, bovine serum albumin, fetal calf serum or protein plasma fraction. The solution containing CD117 placental cells may be mixed and cooled on ice for about 10 minutes. About one ml of DMSO may be added as a cryopreservative. Alternatively, about one ml of an mixture of about 6% HES hydroxyethyl starch and about 5% DMSO may be used as a cryopreservative. The resulting solution is aliquoted into cryovials. Alternatively, the resulting solution may be aliquoted into any container suitable for cryopreservation, such as for example, a cryopreservation bag. The cryovials may then be cryopreserved in a controlled rate freezer (Cryomed) in accordance with controlled rate freezer protocol of the present invention as described herein in further detail. Once the solution containing CD117 placental cells reaches the target temperature of about −90° C., the cryovials are transferred into a long term storage freezer and stored at or below about −135° C. Alternatively, the cryovials or other suitable cryopreservation container may be placed into a monitored dump freeze and frozen to about −80° C. and then transferred into the vapor phase of liquid nitrogen in a long term storage freezer at about −135° C. or less.

Alternatively, the positive fraction may be used to seed culture flasks and culture the cells in accordance with the methods of the present invention. The CD117 placental cells may then be selected from the cell cultures and cryopreserved in accordance with the methods of the present invention as shown, for example, in FIGS. 22c and 22g. A further alternative may be that the cells are concentrated for CD117 by other suitable methods to separate cells such as negative depletion, other positive selections that incorporate the CD117 surface marker, aldehyde dehydrogenase separation, filtration, starch separation, centrifugation techniques including automated processing with centrifugation (i.e., Sepax, Biosafe) and serum deprivation. Such concentration methods may be used throughout the practice of the invention where concentration of cells is necessary.

Preparation for Cell Culture

The population of cells collected in accordance with the methodologies of the present invention may be cultured to further select CD117 placental cells from the cell culture. The population of cells may be prepared for cell culture after concentration according to the present invention or after being cryopreserved and thawed. When applicable, the thawing step comprises preparing aliquots of about 15 ml of density gradient media (Histopaque—Sigma-Aldrich or other suitable media) at about room temperature for each vial containing about 5 ml of cryopreserved cells to be thawed; and then preparing about 25 ml aliquots of Chang's complete media, DMEM complete media or other suitable media for each vial containing about 5 ml of cryopreserved cells to be thawed.

Chang's complete media comprises about 325 ml of MEM alpha media available through Gibco as product 12571-063, about 90 ml of Chang B (basal) C110 (18% v/v) available through Irvine Scientific, about 10 ml of Chang medium C from Supplement C106 (2% v/v) available through Irvine Scientific, about 5 ml Penicillin/Streptomycin (liquid prepared with 10,000 units/ml Penicillin G Sodium and 10,000 ug/ml Streptomycin sulfate in 0.85% saline available through Gibco as product 15140-122, about 5 ml of L-glutamine 200 mM (100×) available through Gibco as product 25030-081, and about 75 ml of ES-Fetal Bovine Serum (15% v/v) available through Gibco as product 10439-024.

If thawing of a cryopreserved sample is necessary, the cryopreserved cells are thawed by removing the vials from the vapor phase of the liquid nitrogen storage freezer. The vials are placed in about a 37° C. to 40° C. water bath and agitated. The cells should not be allowed to completely thaw, but the vials may contain ice. The thawed cells may be diluted by placing the about 5 ml aliquot into the about 25 ml aliquot of chilled Chang's complete media containing about one mg of DNAse available through Pulmozyme.

Alternatively, and if thawing is not necessary, such as for example, when the population of cells is cultured in the absence of the step of cryopreservation, the cellular suspension may be diluted by placing the about 5 ml aliquot into the about 25 ml aliquot of chilled Chang's complete media containing about one mg of DNAse available through Pulmozyme.

The diluted cell suspension may be mixed by inversion. The suspension is centrifuged at about 840 g for about 7 minutes. The supernatant may be aspirated while not disturbing the pellet. The pellet may be brought up to a total volume of about 30 ml Chang's complete media. A small amount of the cell suspension is removed for analysis that includes cell count with a hemocytometer and viability testing using trypan blue or other suitable viability testing methodology. The about 30 ml suspension is overlaid on a density gradient solution available as Histopaque through Sigma-Aldrich or other suitable media, and is centrifuged at about 420 g for about 30 minutes without a brake. The tube is removed from the centrifuge without disrupting the buffy coat. The supernatant is aspirated and the buffy coat is collected. The buffy coat may be brought up to about 20 ml with Chang's complete media and may be washed at about 840 g for about 7 minutes. The supernatant is aspirated, and the pellet may be suspended in Chang's complete media up to about 10 ml, but may also up to about 20 ml or about 30 ml, or even less than about 10 ml. An aliquot of the suspension, such as for example, about 100 ul, may be removed to perform cell count and viability analysis.

Cell Culture

Cells in suspension may be seeded, such as for example at about 40,000 cells/cm², into an untreated tissue culture flask in Chang's complete media, DMEM complete media (with high glucose or low glucose), or other suitable media. The flask may be incubated in about 5% CO₂ in a CO₂ incubator available through Thermo Electron Corp. or Bioscience Technologies, or any other suitable incubator system at a temperature of about 37° C. The cell cultures are monitored for turbidity and pH change. If the pH is high, about 50% of the media should be changed.

The flask may be incubated initially for about 7 days or until the media is significantly out of range as noted by the color of the phenol red indicator in the media. If the pH remains stable after about 7 days, the media may be changed with fresh media (also referred to herein as “virgin media”), as necessary. Only about half of the media may be changed to maintain continuous factors that have been excreted into the media by the cells. After the media change at day 7, the cells may become confluent by day 8 to day 21. Once attaining about 70-80% confluence, the cells may be sub-cultured. Cell cultures are sub-cultured using the trypsin-like enzyme such as TrypLE™ Express available through Gibco, or any other suitable enzyme to provide enough cells to perform the CD117 placental cell selection in accordance with the present invention. For example, cell selection may occur with about 10 million cells. Cell selection may also occur with greater than or less than about 10 million cells.

In accordance with the invention, CD117 placental cells may be collected from the cell culture at a suitable time. In order to collect the CD117 placental cells, adherent cells may be dissociated from the flask. In order to dissociate the cells from the flask, the media is aspirated via an automated pipette. The flask is then rinsed with about 5 ml of Phosphate Buffered Saline (PBS) without calcium or magnesium. The PBS is then removed from the flask with attached cells that have been washed at least once. About one ml of a Trypsin-like recombinant enzyme such as TrypLE™ Express available through Gibco, or any other suitable enzyme, should be added, preferably pre-warmed at about 37° C., to the cell culture in the flask. The flask is agitated to coat the cells with the enzyme. The flask with enzyme may be incubated for about 5 minutes at about 37° C. After incubation, the flask should be gently tapped on a solid surface to dislodge the cells. The flask should be diluted with about 2 ml of Chang's complete media, and the cells transferred to a 15 ml centrifuge tube for washing with Chang's complete media, DMEM complete media (with high glucose or low glucose) or other suitable media. The tube should be centrifuged for about 7 minutes at about 100 g. The supernatant is aspirated and discarded. The pellet may be suspended in a suitable volume of Chang's complete media, DMEM complete media (with high glucose or low glucose) or other suitable media.

At this point, the CD117 placental cells may be selected from the cell culture in accordance with CD117 cell selection methodologies of the present invention. Once selected, the CD117 placental cells may be plated on a Petri dish, seeded into a tissue culture flask or cryopreserved in accordance with present invention.

The cells may be plated in a 9 cm² Petri dish using Chang's complete media (about 15% FBS). Alternatively, the cells may be placed in a tissue culture flask with a vented cap. If the pH of the media becomes high, the cells may be washed with Chang's complete media. When necessary after suitable growth, the cells may be dissociated from the Petri dish or tissue culture flask using a trypsin-like enzyme and then placed in an untreated tissue culture flask using Chang's complete media. After suitable growth, the cells may be dissociated using a trypsin-like enzyme such as TrypLE™ Express available through Gibco and then seeded in a fresh untreated tissue culture flask. This process may be repeated in order to maintain desired cell growth. The cells may be washed with fresh media, or about 50% of the media or other suitable amount may be replaced with fresh media if the pH of the media is high. At this point, the CD117 placental cells may be selected from the cell culture in accordance with the CD117 cell selection methodology of the present invention. The selected CD117 placental cells may be plated on a Petri dish, seeded into a tissue culture flask or cryopreserved in accordance with present invention.

Cell Lines

Several fetal placental cell lines have been developed from the practice of the methods, processes and systems of the present invention.

Cell Line 1005R P3

A whole mammalian placenta was delivered and a tincture of iodine was placed on the fetal side of the placenta in accordance with the invention. Two pieces of placental tissue measuring about 6 cm by about 12 cm square and cut through the depth of the organ from fetal side to maternal side were obtained from the whole placenta through use of a sterile scalpel and forceps provided in a tissue collection kit. The DPBS of a tissue collection kit was poured into the sterile container of the tissue collection kit, and the pieces of placental tissue were submerged in the DPBS. The sterile container was closed, chilled and packaged for shipment in accordance with the present invention and then shipped to the processing facility. The pieces of placental tissue arrived at the processing facility within about 15 hours after delivery of the whole placenta and collection of the pieces of placental tissue. The sterile container with the tissue samples was received at the processing facility and unpackaged from the shipping box and logged into the batch record.

The sterile container containing the DPBS and piece of placental tissue was disinfected, transferred into a clean room, and placed on ice in an ice pan. The sterile container was placed in a BSC and the top of the sterile container was removed. A sample of the DPBS buffer (about 4 ml) was removed from the sterile container using a sterile syringe and used to inoculate BacT/ALERT blood culture bottles for detecting aerobic and anaerobic bacteria and fungal organisms. The blood culture bottles were incubated at about 37° C. in a BacT/ALERT system for about 7 days, whereby the results indicated a positive identification of E. coli and Streptococcus viridans as shown in Table 1.

TABLE 1
BacT/ALERT System Analysis

| Status | Type | Loaded |
|---|---|---|
| POSITIVE - Escherichia | BTA PF, aerobic | Nov. 21, 2006 at 18:03 |
| coli | | |
| POSITIVE - Streptococcus | BTA, anaerobic | Nov. 21, 2006 @ 18:03 |
| viridans | | |

The two pieces of placental tissue were removed from the sterile container for decontamination. Prior to disinfection, the fetal placental membranes of the pieces of placenta were physically separated from portions of the maternal membrane of the piece of placenta using forceps and a scalpel, and the portions of the maternal membrane was discarded.

The placental tissue was then subjected to a disinfection process in the BSC. The placental tissue was initially rinsed in about 50 ml of DPBS and then was disinfected by dipping it into a container of about 150 ml of about 50% mixture of Betadine (10% Povidone-iodine Topical Solution—Purdue Products) and about 50% PBS buffer for about 5 seconds and removed. The placental tissue was then disinfected by dipping it in a container of about 150 ml of a solution of several antibiotics for about 3 minutes to kill a broad range of pathogens. The antibiotic solution comprised three antibiotics including Amphotericin B, Streptomycin, and Cephazolin (X-Gen Pharmaceuticals Inc.) mixed with a buffer HBSS. The antibiotic solution was prepared by suspending about 50 mg of each antibiotic in about 10 ml of HBSS. The suspended antibiotics were added to about 100 ml of HBSS. Finally, the placental membrane was rinsed by dipping it in DPBS (1× without Calcium and Magnesium—Cellgro) for about 5 seconds to about 15 seconds.

The disinfected pieces of placental tissue were then transferred to a sterile container with DPBS (1× without Calcium and Magnesium—Cellgro). All of the steps of tissue separation and decontamination were performed at room temperature of about 18° C.

The sterile container with disinfected placental tissue was placed on ice in preparation for performing the subsequent steps of the invention.

The placental tissue was removed from the sterile container and placed in a wash solution in a Petri dish. The wash solution comprised about 500 ml of HBSS without Calcium chloride and Magnesium chloride (Gibco), about 5 ml of Heparin (Heparin Sodium 1,000 USP Units/ml—American Pharmaceutical Partners), about 2.5 ml of DNase (Genentech 2.5 mg/ml), and about 50 ml of protein (25% Human serum albumin HSA—Telacris Biotherapeutics). The wash solution was chilled on ice. The placental tissue in the wash was minced with a scalpel and forceps to the smallest possible pieces, which were suspended in the wash solution in a Petri dish.

Enzyme digestion of the minced pieces of placental tissue occurred in one step. About 4.9 g of minced placental tissue was placed in a digestion solution of about 10 ml in a tube and incubated at about 37° C. in an incubator for about 45 minutes. After incubation, the placental tissue was removed from the digestion solution in the tube and discarded. The enzymatic reaction in the tube was stopped by adding about 4 ml of 25% HSA (Baxter with concentrations of 1000 units per ml) and mixing the digestion solution with the HSA. The solution was filtered through a 100 micron filter into 50 ml collection tubes. The 50 ml collection tubes were centrifuged at about 2000 rpm for about 7 minutes at about 20° C. The supernatant was removed from the collection tubes and the pelleted cells were re-suspended in about 45 ml of wash solution. The re-suspended cells were filtered through a 100 micron filter and into a 50 ml collection tube. The filter was rinsed with wash solution. The filtered, re-suspended cells and wash solution were combined into a 50 ml collection tube and wash solution was added to bring the total volume up to about 35 ml. The collection tube was centrifuged at about 2000 rpm for about 7 minutes at about 20° C.

The placental cells in the wash solution collected in conical collection tubes from either mechanical separation or enzymatic digestion were subjected to centrifugation to concentrate the placental cells. The wash solution containing placental cells were equally separated into conical tubes for centrifugation. Centrifugation comprised subjecting the placental cells suspended in the wash solution to about 2000 rpm for about 7 minutes between about 2° C. to about 30° C. The conical collection tubes were removed from the centrifuge and the supernatant was aspirated with suction and discarded. A suitable volume of wash solution was used to re-suspend the pelleted cells in each tube. About 2 ml to about 3 ml of wash solution was used to re-suspend the pelleted cells. All of the re-suspended placental cells in the wash solution were transferred into a 50 ml conical collection tube, and the volume of the cellular suspension in the conical collection tube was brought up to a total volume of about 31 ml with the wash solution. About 100 ul of the cellular suspension was removed and placed in a pre-processing collection tube for testing the total nucleated cell count and viability with Trypan blue. The TNC was about 1.73 million cells.

The cellular suspension was concentrated through centrifugation. The suspension was centrifuged in the conical collection tube at about 2000 rpm for about 7 minutes at about 20° C.

Bacteriological analysis of the supernatant was performed using the BacT/ALERT system for microbial analysis. A new sterile alcohol pad was opened and placed on top of the septum of the culture bottle. A sterile syringe used to collect between about 4 ml of supernatant from the conical collection tube after centrifugation and to inoculate the BacT/ALERT blood culture bottles for detecting aerobic bacteria and fungus. The blood culture bottle was incubated at about 37° C. in a BacT/ALERT system for about 7 days, whereby the results were negative identification for bacteria and fungus growth as shown in Table 2.

TABLE 2
BacT/ALERT System Analysis

| Status | Type | Loaded |
|---|---|---|
| NEGATIVE | BTA PF | Nov. 21, 2006 @ 18:03 |

The pelleted cells were re-suspended with wash solution up to about 6 ml. About 100 ul of cellular suspension was removed and placed into a post-processing tube and tested for the total nucleated cell count.

The cells were diluted by placing about 5 ml into the about 25 ml aliquot of chilled Chang's complete media containing about one mg of DNAse (Pulmozyme).

The diluted cell suspension was mixed by inversion. The suspension was centrifuged at about 840 g for about 7 minutes at about 4° C. The supernatant was aspirated without disturbing the pellet. The pellet was brought up to a total volume of about 7 ml Chang's complete media.

The cells in suspension were seeded at about 42,000 cells/cm² into an untreated tissue culture flask in Chang's complete media. The flask was incubated in about 5% CO₂ in a CO₂ incubator (Thermo Electron Corp.) at a temperature of about 37° C. The cell cultures were monitored for turbidity and pH change.

The flask was incubated initially for about 7 days. All of the media was changed to maintain continuous factors that have been excreted into the media by the cells. After the media change at day 7, the cells became confluent by day 12. Once attaining about 70-80% confluence, the cells were sub-cultured. Cell cultures were sub-cultured using the trypsin-like enzyme such as TrypLE™ Express (Gibco) to provide enough cells to perform the CD117 placental cell selection. The cells were cultured through several passages according to the invention and as summarized in Table 3.

Phenotype and Validity Analysis

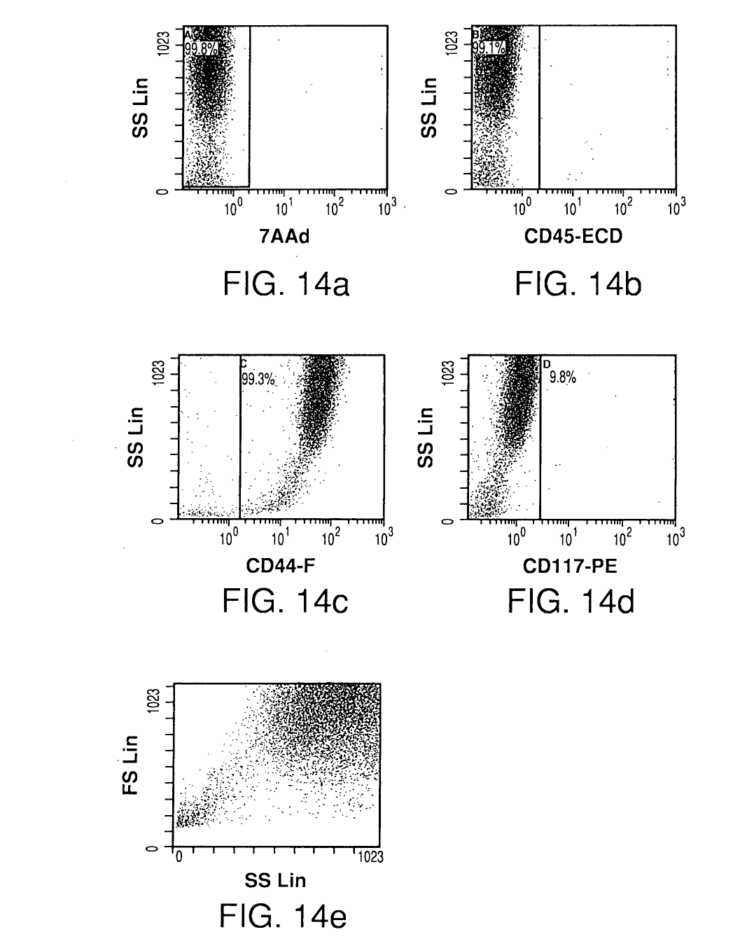

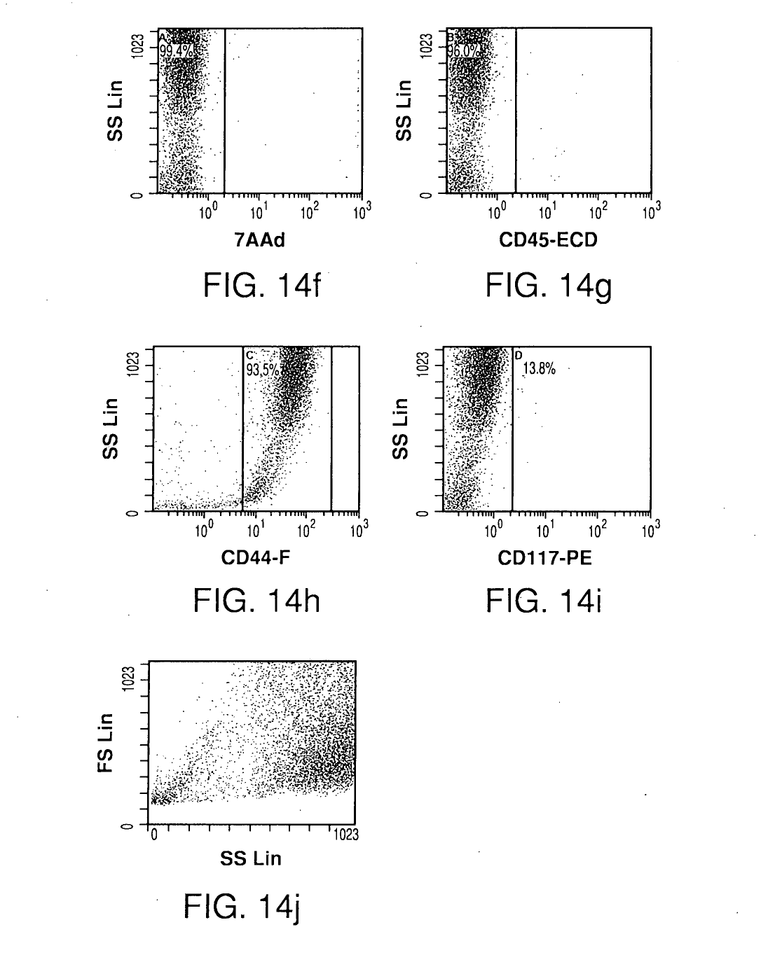

Phenotype analysis was performed in accordance with the methods described herein. The phenotype analysis was performed at different passages of the cell culture for cell surface markers including CD29, CD34, CD44, CD45, CD90, CD 105, CD117 and CD166. The cell phenotyping was performed with commercially-available monoclonal and polyclonal antibodies specific for the aforementioned cell surface markers using suitable flow cytometry analysis. As summarized in Table 3 and at various passages, the data collected from the assessment showed that the cells of Cell Line 1005R P3 expressed CD29, CD44, CD90, CD105, CD117 and CD166 positive and had low or no expression of CD44 with a high percentage of viability throughout the duration of cell culture. The results of the phenotyping and validity assessment are shown in FIGS. 14a through 14j.

TABLE 3
Phenotype and Validity Analysis of Cell Line 1005R P3

| | | CD44- | CD45- | | | | | | | |
|---|---|---|---|---|---|---|---|---|---|---|
| | CD117- | FITC | ECD | 7AAD- | 7AAD- | | | | | |
| Passage # | PE | (POS) | (NEG) | TEST | ISO | CD166 | CD105 | CD29 | CD34 | CD90 |
| 1005R | 1.10% | 98.10% | 98.90% | 99.80% | 99.80% | N/A | N/A | N/A | N/A | N/A |
| A3 P2 | | | | | | | | | | |
| 1005R | 7.50% | 97.90% | 96.80% | 99.90% | 99.90% | 99.30% | 98.80% | 98.30% | 0.50% | 99.60% |
| A3 P3 | | | | | | | | | | |
| 1005R | 9.60% | 98.60% | 99.10% | 99.80% | 100.00% | 99.10% | 98.80% | 98.60% | 1.00% | 99.40% |
| A3 P4 | | | | | | | | | | |
| 1005R | 11.40% | 93.50% | 96.00% | 99.40% | 99.50% | N/A | N/A | N/A | N/A | N/A |
| A3 P6 | | | | | | | | | | |

Genotype Analysis of Cell Line 1000R A3

Human Identification-Multiplex Short Tandem Repeat (STR) Analysis was performed on the Cell Line 1005R A3 and on reference samples of maternal peripheral blood and baby cord blood collected at delivery to determine the origin of the cells of Cell Line 1005R P3.

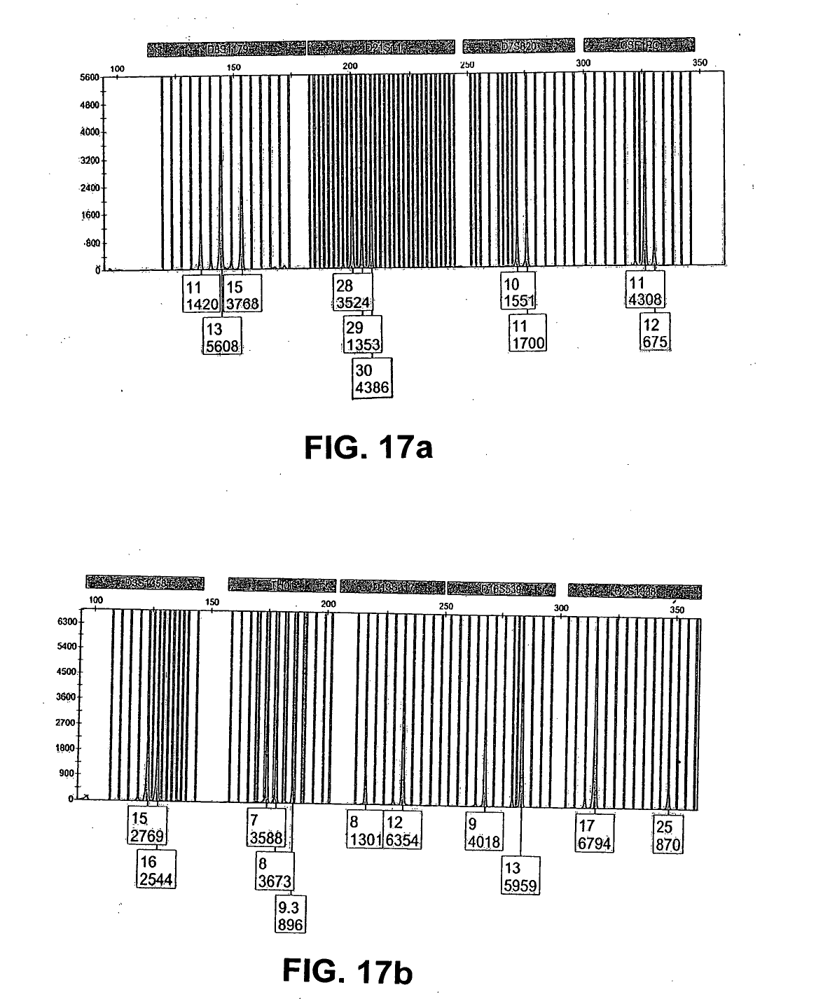

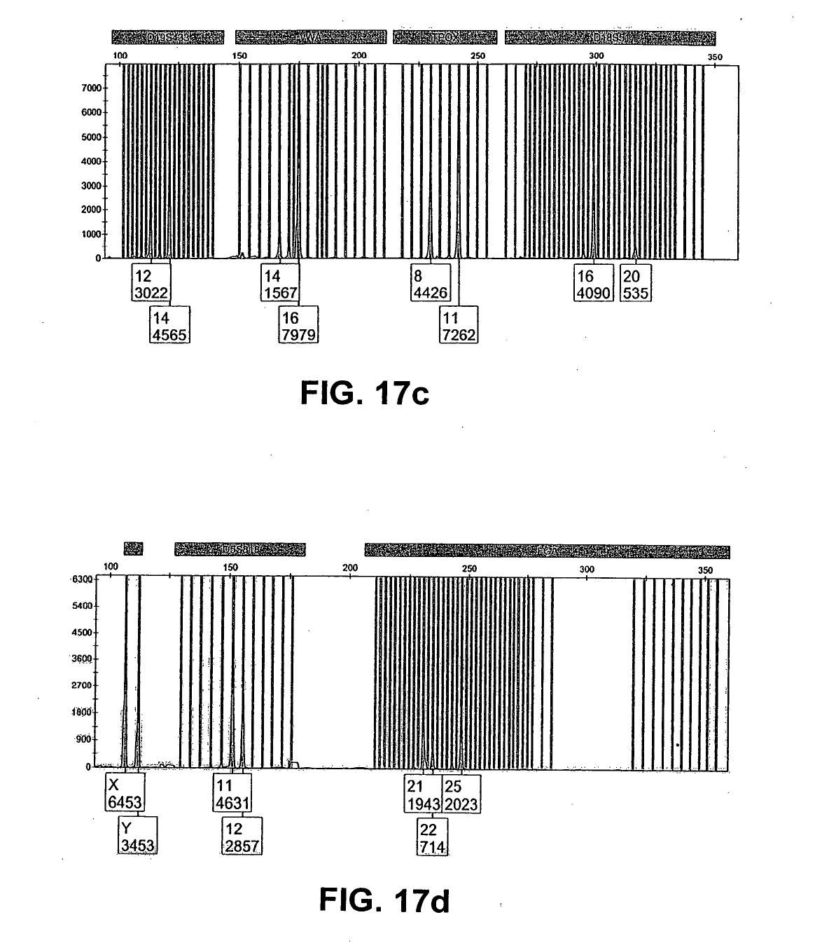

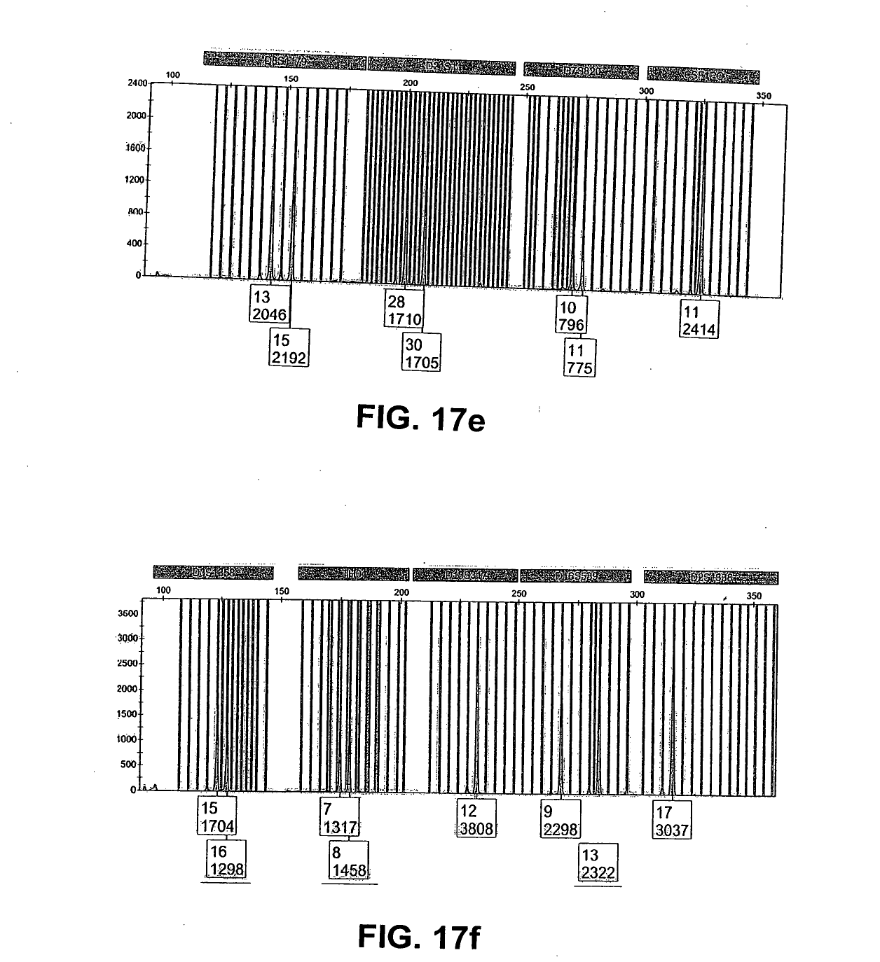

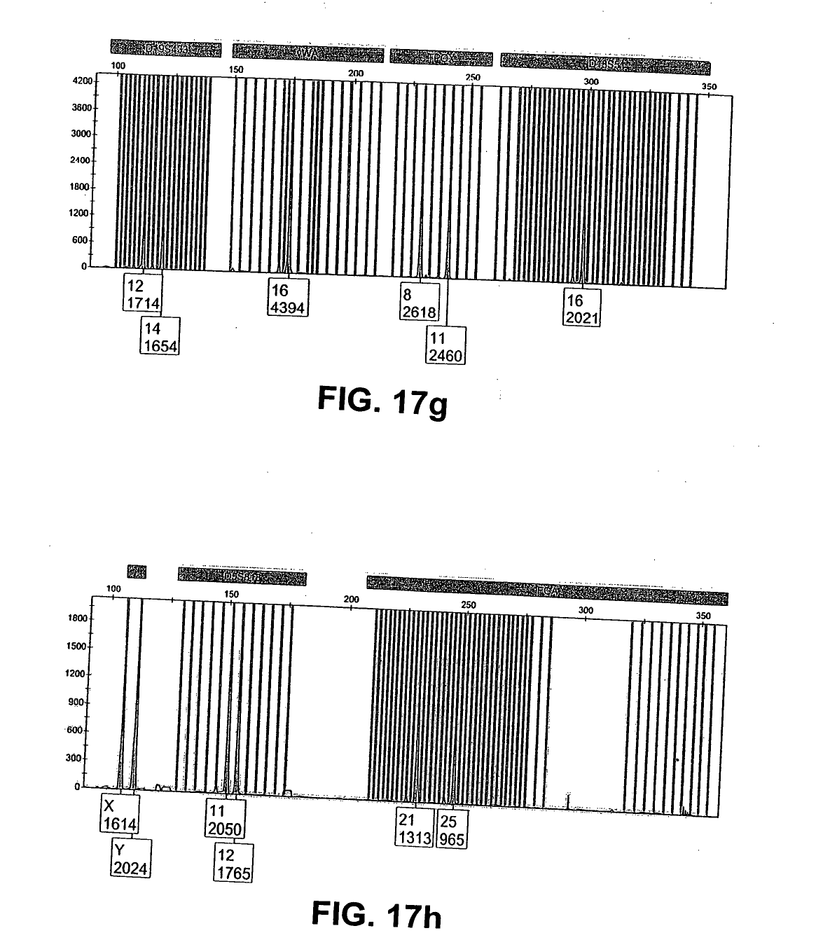

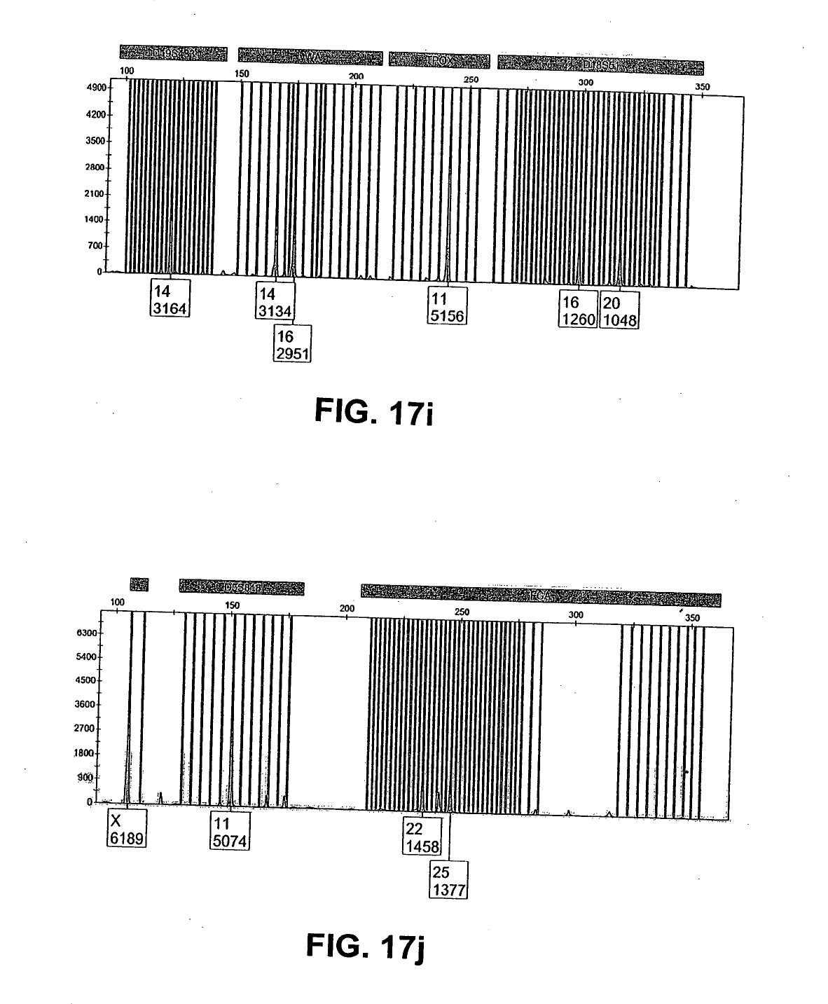

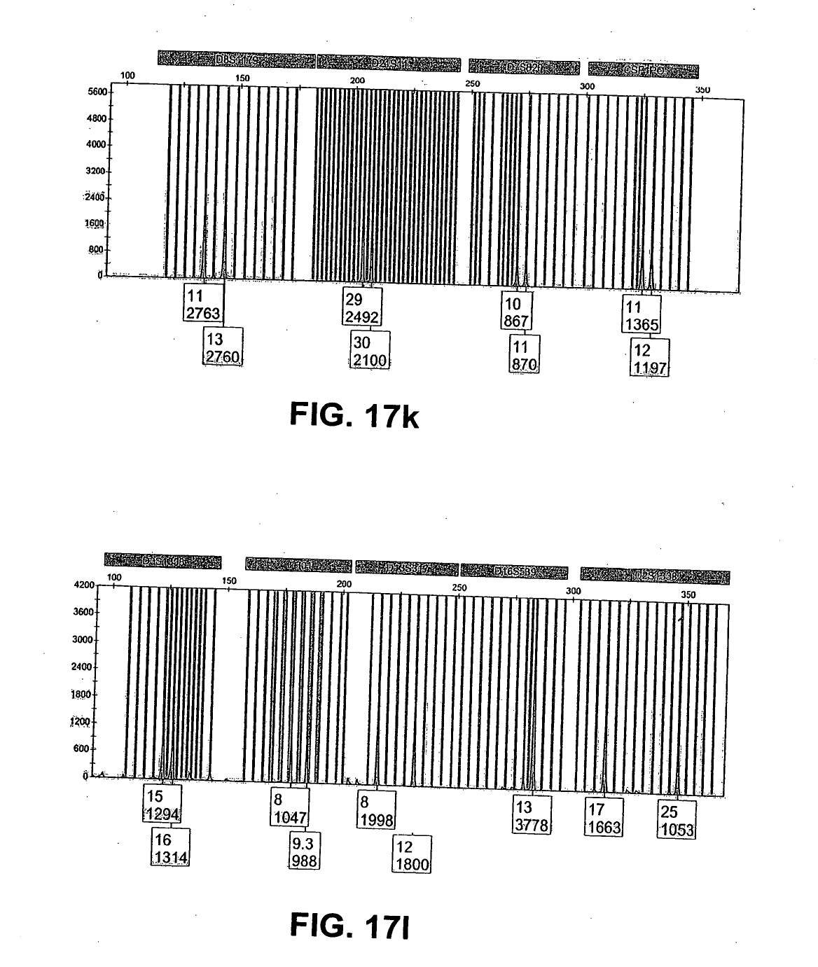

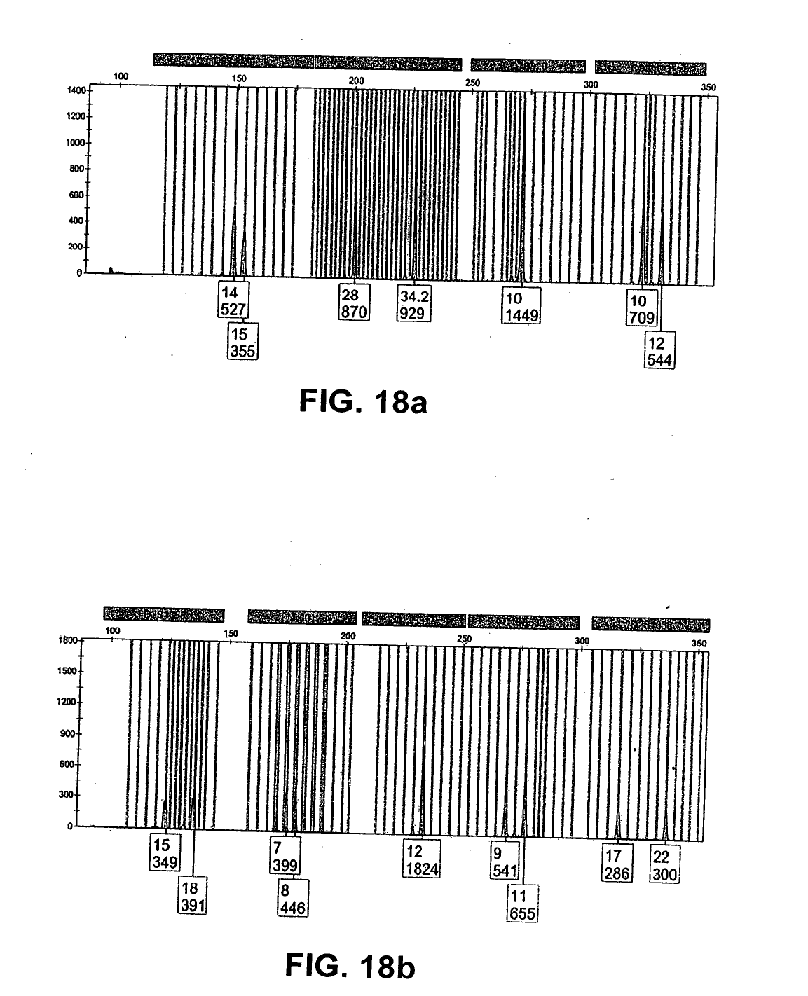

The STR Analysis involved investigating 15 different short tandem repeat (STR) gene regions plus amelogenin on the X and Y chromosomes were simultaneously subjected to PCR and then analyzed. Four separate fluorescent dye labels are used to label the samples. The dyes were coupled to PCR primers. Each of these fluorescent dyes emitted its maximum fluorescence at a different wavelength, that was detected by a Gene Analyzer. The 15 STR loci investigated were D8S1179, D21S11, D7S820, CSF1PO, D3S1358, TH01, D13S317, D16S539, D2S1338, D19S433, vWA, TPOX, D18S51, D5S818, FGA. The amplified product was electrophoresed on ABI 3100 Genetic Analyzer and analyzed using the GeneMapper ID software program. The sensitivity level of the assay to detect mixed chimerism was about 5%. The results of the STR analysis indicated that the 1005R P3 specimen was of single individual origin and was 100% fetal and 0% maternal as shown in FIGS. 17a through 17l.

Cell Line PLF05

A whole mammalian placenta was delivered and a tincture of iodine was placed on the fetal side of the placenta in accordance with the invention. Two pieces of placental tissue measuring about 6 cm by about 12 cm square and cut through the depth of the organ from fetal side to maternal side were obtained from the whole placenta through use of a sterile scalpel and forceps provided in a tissue collection kit. The DPBS of the tissue collection kit was poured into the sterile container of the tissue collection kit and the piece of placental tissue was submerged in the DPBS. The sterile container was closed, chilled and packaged for shipment in accordance with the present invention and then shipped to the processing facility. The piece of placental tissue arrived at the processing facility within about 15 hours after delivery of the whole placenta and collection of the pieces of placental tissue. The sterile container with the tissue sample was received at the processing facility and unpackaged from the shipping box and logged into the batch record.

The sterile container containing the DPBS and piece of placental tissue was disinfected, transferred into a clean room, and placed on ice in an ice pan. The sterile container was placed in a BSC and the top of the sterile container was removed. A sample of the DPBS buffer (about 4 ml) was removed from the sterile container using a sterile syringe and used to inoculate BacT/ALERT blood culture bottles for detecting aerobic and anaerobic bacteria. The blood culture bottles were incubated at about 37° C. in a BacT/ALERT system for about 7 days, whereby the results indicated a positive identification for coagulase negative Staphylococcus epidermidis and Enterococcus faecalis as shown in Table 4.

TABLE 4
BacT/ALERT System Analysis

| Status | Type | Loaded |
|---|---|---|
| POSITIVE - COAGULASE | BTA PF | Dec. 22, 2006 @ 5:08 |
| NEG. STAPH. EPIDERMIDIS | | |
| POSITIVE - ENTEROCOCCUS | BTA | Dec. 22, 2006 @ 5:08 |
| FAECALIS | | |

The pieces of placental tissue were removed from the sterile container for decontamination. Prior to disinfection, the fetal placental membrane of the piece of placenta was physically separated from portions of the maternal membrane of the piece of placenta using forceps and a scalpel, and the portions of the maternal piece of the placenta was discarded. Placental tissue from the fetal side including the amniochorion layer and excluding all decidual tissue, which was scraped away from the amniochorion tissue, was used for processing.

The placental tissue was then subjected to a disinfection process in the BSC. The placental tissue was initially rinsed in about 50 ml of DPBS and then was disinfected by dipping it into a container of about 150 ml of about 50% mixture of Betadine (10% Povidone-iodine Topical Solution—Purdue Products) and about 50% PBS buffer for about 5 seconds and removed. The placental tissue was then disinfected by dipping it in a container of about 150 ml of a solution of several antibiotics to kill a broad range of pathogens for about 3 minutes. The antibiotic solution comprised three antibiotics including Amphotericin B, Streptomycin, and Cephazolin (X-Gen Pharmaceuticals Inc.) mixed with a buffer HBSS. The antibiotic solution was prepared by suspending about 50 mg of each antibiotic in about 10 ml of HBSS. The suspended antibiotics were added to about 100 ml of HBSS. Finally, the placental membrane was rinsed by dipping it in DPBS (1× without Calcium and Magnesium—Cellgro) for about 5 seconds to about 15 seconds.

The disinfected pieces of placental tissue were then transferred to a sterile container with DPBS (1× without Calcium and Magnesium—Cellgro). All of the steps of tissue separation and decontamination were performed at room temperature of about 18° C.

The sterile container with disinfected placental tissue was placed on ice in preparation for performing the subsequent steps of the invention. The portions of the placental tissue that was removed or residual from tissue processing, the washes and antibiotic solutions were discarded as medical waste.

The placental tissue was removed from the sterile container and placed in a wash solution in a Petri dish. The wash solution comprised about 500 ml of HBSS without Calcium chloride and Magnesium chloride (Gibco), about 5 ml of Heparin (Heparin Sodium 1,000 USP Units/ml—American Pharmaceutical Partners), about 2.5 ml of DNase (Genentech 2.5 mg/ml), and about 50 ml of protein (25% Human serum albumin HSA—Telacris Biotherapeutics). The wash solution was chilled on ice. The placental tissue in the wash was minced with a scalpel and forceps to the smallest possible pieces, which were suspended in the wash solution in a Petri dish.

Enzyme digestion of the minced pieces of placental tissue occurred in sequential steps. About 14.6 g of minced placental tissue was subjected to the sequential steps of enzymatic digestion according to the following process. About 6 grams of placental tissue was placed in a first digestion solution of about 10 ml in a first tube. The first tube was incubated in a sterile bag at about 37° C. in an incubator for about 10 minutes at about 37° C. The placental tissue was removed from the digestion solution in the first tube and then placed in a second tube with about 10 ml of new digestion solution. The digestion solution in the first tube was discarded. The second tube with digestion solution and placental tissue was incubated in a sterile bag, at about 37° C. in an incubator for about 20 minutes. The tube was inverted at about 10 minutes. The placental tissue was removed from the second tube and placed in a third tube containing about 10 ml of new digestion solution. The enzymatic reaction in the enzyme digestion solution in the second tube was stopped by adding about 4 ml of 25% HSA (Baxter with concentrations of 1000 units per ml) and mixing the digestion solution with the HSA. The second tube was placed on ice.

The third tube containing about 10 ml of digestion solution and placental tissue was incubated in a sterile bag at 37° C. in an incubator for about 20 minutes. The tube was inverted at about 10 minutes. The placental tissue was removed from the third tube and placed in a fourth tube containing 10 ml of digestion solution. The enzymatic reaction in the third tube was stopped by adding about 4 ml of 25% HSA (Baxter with concentrations of 1000 units per ml) and mixing the digestion solution with the HSA. The third tube was placed on ice.

The fourth tube containing about 10 ml of digestion solution and placental tissue was incubated in a sterile bag at 37° C. in an incubator for about 20 minutes. The tube was inverted at about 10 minutes. The placental tissue was removed from the fourth tube and placed in a fifth tube containing about 10 ml of digestion solution. The enzymatic reaction in the fourth tube was stopped by adding about 4 ml of 25% HSA (Baxter with concentrations of 1000 units per ml) and mixing the digestion solution with the HSA. The fourth tube was placed on ice.

The fifth tube containing about 10 ml of digestion solution and placental tissue was incubated in a sterile bag at 37° C. in an incubator for about 20 minutes. The tube was inverted at about 10 minutes. The placental tissue was removed from the fifth tube and discarded. The enzymatic reaction in the fifth tube was stopped by adding about 4 ml of 25% HSA (Baxter with concentrations of 1000 units per ml) and mixing the digestion solution with the HSA. The fifth tube was placed on ice.

The digestion solution containing placental cells in the second through fifth tubes were filtered through a 100 micron filter. About 10 ml of wash solution was used to wash and rinse the contents of the second through fifth tubes into the filter and to wash and rinse the filter. The filtered digestion solution and wash solution was collected in 50 ml collection tubes. The 50 ml collection tubes were centrifuged at about 2000 rpm for about 7 minutes at about 20° C. The supernatant was removed from the collection tubes and the pelleted cells were re-suspended in about 45 ml of wash solution. The re-suspended cells were filtered through a 100 micron filter and into a 50 ml collection tube. The filter was rinsed with wash solution. The filtered, re-suspended cells and wash solution were combined into a 50 ml collection tube and wash solution was added to bring the total volume up to about 35 ml.

The placental cells in the wash solution collected in conical collection tubes from enzymatic digestion were subjected to centrifugation to concentrate the placental cells. The wash solution containing placental cells were equally separated into conical tubes for centrifugation. Centrifugation comprises subjecting the placental cells suspended in the wash solution to about 2000 rpm for about 7 minutes at about 20° C. The conical collection tubes were removed from the centrifuge and the supernatant was aspirated with suction and discarded. A suitable volume of wash solution was used to re-suspend the pelleted cells in each tube. About 2 ml to about 3 ml of wash solution was used to re-suspend the pelleted cells. All of the re-suspended placental cells in the wash solution were transferred into a 50 ml conical collection tube, and the volume of the cellular suspension in the conical collection tube was brought up to a total volume of about 31 ml with the wash solution. About 100 ul of cellular suspension was collected and used for total cell count and viability analysis. The TNC was about 925,000 cells with 100% viability.

The cellular suspension was then concentrated through centrifugation. The suspension was centrifuged in the conical collection tube at about 2000 rpm for about 7 minutes at about 20° C.

Bacteriological analysis of the supernatant was performed using the BacT/ALERT system. A new sterile alcohol pad was opened and placed on top of the septum of the culture bottle. A sterile syringe used to collect between about 4 ml of supernatant from the conical collection tube after centrifugation and to inoculate the BacT/System blood culture bottles for detecting aerobic and anaerobic bacteria. The blood culture bottles were incubated at about 37° C. in a BacT/ALERT system for about 7 days, whereby the results were negative as shown in Table 5.

TABLE 5
BacT/ALERT System Analysis

| Status | Type | Loaded |
|---|---|---|
| NEGATIVE | BTA PF | Dec. 22, 2006 @ 5:08 |
| NEGATIVE | BTA | Dec. 22, 2006 @ 5:08 |

The pelleted cells were re-suspended with wash solution up to about 6 ml. About one ml of cellular suspension was removed and placed into a post-processing tube and was tested for the total nucleated cell count, cell viability and flow cytometric analysis for CD117 and other cell surface markers. About 5 ml of suspension was further processed in preparation for cryopreservation.

The about 5 ml of cellular suspension obtained by centrifugation was combined with a cryopreservation agent in preparation for cryopreservation. The cryopreservation agent comprised about 3 ml of the buffer DPBS, about one ml of the protein HSA (Telacris Bio) and about one ml of the preservative DMSO (99% Stemsol). The cryopreservation agent was carefully added to the about 5 ml of cellular suspension to a total volume of about 10 ml of mixture of cellular suspension and cryopreservation agent. The mixture was separated into two 5 ml aliquot volumes in cryovials in several vials adapted for cryopreservation.

The cryovials with the placental cells and cryopreservation agent were subjected to several temperature reduction steps to reduce the temperature of the placental cells comprising fetal placental cells to a final temperature of about −90° C. with a controlled rate freezer (Cryomed Thermo Form a Controlled Rate Freezer 7454 (Thermo Electron, Corp.)). The following temperature reduction steps were programmed in the controlled rate freezer: first reducing the mixture of the placental cells and cryopreservation agent to about 4° C. and then reducing the mixture at about 1° C. per minute to about −3° C., and then about 10° C. per minute to about −20° C., and then about 1° C. per minute to about −40° C., and finally about 10° C. per minute to about −90° C. The cryovials containing the mixture of the population of cells and cryopreservation agent were placed in the controlled rate freezer and subjected to the temperature reduction steps. Once the mixture and cryopreservation agent reached about −90° C., the cryopreservation vials were transferred to a cryogenic storage unit and stored in the vapor of liquid Nitrogen at a temperature at or below about −135° C. in a LN2 Freezer MVE 1830 (Chart Industries).

The cryopreserved placental cells were prepared for cell culture after being thawed. The thawing step comprised preparing aliquots of about 15 ml of LSM density gradient media (Histopaque—Sigma-Aldrich) at about room temperature for each vial containing about 5 ml of cryopreserved cells to be thawed; and then preparing about 25 ml aliquots of Chang's complete media for each vial containing about 5 ml of cryopreserved cells to be thawed.

The cryopreserved cells were thawed by removing the vials from the vapor phase of the liquid nitrogen storage freezer. The vials were placed in about a 37° C. to 40° C. water bath and agitated. The cells were not allowed to completely thaw, but the vials contained ice. The thawed cells were diluted by placing the about 5 ml aliquot into the about 25 ml aliquot of chilled Chang's complete media containing about one mg of DNAse available through Pulmozyme.

The diluted cell suspension were mixed by inversion. The suspension was centrifuged at about 840 g for about 7 minutes at about 4° C. The supernatant was aspirated without disturbing the pellet. The pellet was brought up to a total volume of about 30 ml with Chang's complete media. A small amount of the cell suspension was removed for analysis that included cell count with a hemocytometer and viability testing using trypan blue.

The cells in suspension were seeded at about 3600 cells/cm² into an untreated tissue culture flask in Chang's complete media. The flask was incubated in about 5% CO₂ in a CO₂ incubator (Thermo Electron Corp) at a temperature of about 37° C. The cell cultures were monitored for turbidity and pH change.

The flask was incubated initially for about 7 days. All of the media was changed to maintain continuous factors that have been excreted into the media by the cells. After the media change at day 7, the cells became confluent by day 19. Once attaining about 70-80% confluence, the cells may be sub-cultured. Cell cultures are sub-cultured using the trypsin-like enzyme such as TrypLE™ Express (Gibco) to provide enough cells to perform the CD117 placental cell selection. The cells were cultured for several passages according to the present invention and as summarized in Table 6.

CD117 placental cells were collected from the cell culture at passage 6. In order to collect the CD117 placental cells, adherent cells were dissociated from the flask. The flask was rinsed with about 5 ml of Phosphate Buffered Saline (PBS) without calcium or magnesium. The PBS was removed from the flask with attached cells washed at least once. About one ml of a Trypsin-like recombinant enzyme (TrypLE™ Express—Gibco) was added to the cell culture in the flask. The flask was agitated to coat the cells with the enzyme. The flask with enzyme was incubated for about 5 minutes at about 37° C. After incubation, the flask was gently tapped on a solid surface to dislodge the cells. The flask was diluted with about 2 ml of Chang's complete media, and the cells transferred to a 15 ml centrifuge tube for washing with Chang's complete media. The tube was centrifuged for about 7 minutes at about 100 g at about 20° C. The supernatant was aspirated and discarded. The pellet was suspended in a suitable volume of Chang's complete media.

The cellular suspension was centrifuged at about 300 g for about 10 minutes at about 20° C. The pellet was suspended in a working buffer with anti-human CD117 antibodies. The working buffer comprised PBS at about pH 7.2, bovine serum albumin, EDTA and about 0.09% Azide (BD Biosciences). The pellet was suspended in about 100 ul of working buffer and about 5 ug of purified monoclonal mouse anti-human CD117 antibody (available as 104D2 from Santa Cruz or YB5.58 from BD Biosciences).

The solution comprising the cells, working buffer and anti-CD117 antibodies were incubated for a first incubation period of about 20 minutes to about 25 minutes on ice. After the incubation period, the solution with the cells was washed with working buffer to remove unbound antibody and then centrifuged at about 300 g for about 10 minutes at about 4° C. After centrifugation, the supernatant was aspirated and was saved for analysis, and the pellet was suspended in working buffer.

A second batch of antibodies having microbeads affixed thereto and having an affinity for the goat anti-mouse IgG antibody are added to the working buffer used to suspend the pellet. The microbeads comprised iron oxide and polysaccharide (Miltenyi Biotec-130-048-401). A 2 ml vial of the aforementioned goat anti-mouse IgG was used for approximately 1.0×10e9 of total un-separated cells.

The cellular suspension was incubated for a second incubation period of about 30 minutes at about 4° C. After the incubation period was complete, the cells were washed with working buffer, and the cells were centrifuged. The supernatant was aspirated and saved for analysis, and the pellet containing cells was suspended in about 500 ul of working buffer.

The CD117 cells were separated from the cellular suspension in working buffer using a MS column (Miltenyi Biotec) to separate the CD117 placental cells. The MS column was prepared by rinsing it with working buffer. The column was placed in a magnetic field of a MACS separator (Miltenyi Biotec).

Unlabeled cells flowed through the column and were collected in a sterile tube for cell phenotyping and cell count. The unlabeled cells were identified as a negative fraction. The column was washed with working buffer after adding the cellular suspension. The effluent from the washing steps may be collected for cell phenotyping and count. The effluent was identified as a negative fraction.

The labeled CD117 placental cells were collected from the column after the column was washed. The labeled CD117 placental cells were collected by placing a sterile tube under the column and removing the column from the magnetic field. Once the column was removed from the magnetic field, the labeled CD117 placental cells passed through the column and into the sterile tube. Residual labeled CD117 placental cells in the column were washed out by adding working buffer to the column to wash the cells through the column and by stripping the column with a plunger to release the cells. The collected labeled CD117 placental cells were identified as the positive fraction. The positive fraction was centrifuged at about 300 g for about 10 minutes at about 4° C. and the supernatant aspirated. The pellet was suspended in about 5 ml of working buffer.

The positive fraction and the negative fraction were analyzed with a hemocytometer to obtain a total count of viable cells. The negative fraction is analyzed by flow cytometry for phenotyping. The TNC of the positive fraction was about 75,000 cells with 95% viability by trypan blue analysis. The TNC of the negative fraction was 16 million cells with 100% viability by trypan blue analysis.

The cells in suspension were seeded at about 3000 cells/cm² into an untreated tissue culture flask in Chang's complete media. The flask was incubated in about 5% CO₂ in a CO₂ incubator (Thermo Electron Corp) at a temperature of about 37° C. The cell cultures are monitored for turbidity and pH change.

The flask was incubated initially for 4 days. After the media change at day 4, the cells became confluent. All of the media was changed to maintain continuous factors that have been excreted into the media by the cells. After the media change at day 4, the cells became confluent. The cell culture of the positive fraction of cells went through several passages of culture according to the present invention.

CD117 placental cells were collected from the cell culture at passage 1. In order to collect the CD117 placental cells, adherent cells were dissociated from the flask. In order to dissociate the cells from the flask, the media was aspirated via an automated pipette. The flask was rinsed with about 5 ml of Phosphate Buffered Saline (PBS) without calcium or magnesium. The PBS was removed from the flask with attached cells that have been washed at least once. About one ml of a Trypsin-like recombinant enzyme such as TrypLE™ Express available through Gibco was added, pre-warmed at about 37° C., to the cell culture in the flask. The flask was agitated to coat the cells with the enzyme. The flask with enzyme was incubated for about 5 minutes at about 37° C. After incubation, the flask was gently tapped on a solid surface to dislodge the cells. The flask was diluted with about 2 ml of Chang's complete media, and the cells transferred to a 15 ml centrifuge tube for washing with Chang's complete media. The tube was centrifuged for about 7 minutes at about 100 g at about 20° C. The supernatant was aspirated and discarded. The pellet was suspended in a suitable volume of Chang's complete media.

The cellular suspension containing CD117 placental cells was prepared for cryopreservation. About one ml of human serum albumin, about 3 ml of DPBS and about one ml of DMSO were added to the about 5 ml of the positive fraction. The solution containing CD117 placental cells was mixed and cooled on ice for about 10 minutes. About one ml of DMSO was added as a cryopreservative. The cryovials were cryopreserved in a controlled rate freezer (Cryomed) in accordance with controlled rate freezer protocol of the present invention as described herein in further detail. Once the solution containing CD117 placental cells reached the target temperature of about −90° C., the cryovials were transferred into a long term storage freezer and stored at about −135° C. or less.

Phenotype and Validity Analysis of Cell Line PLF05

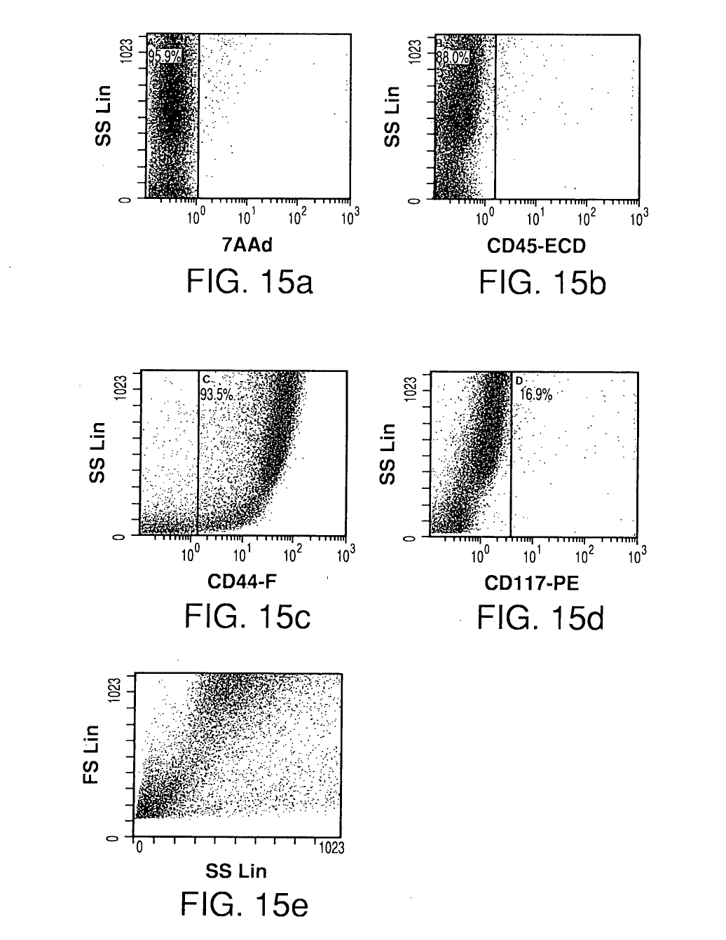

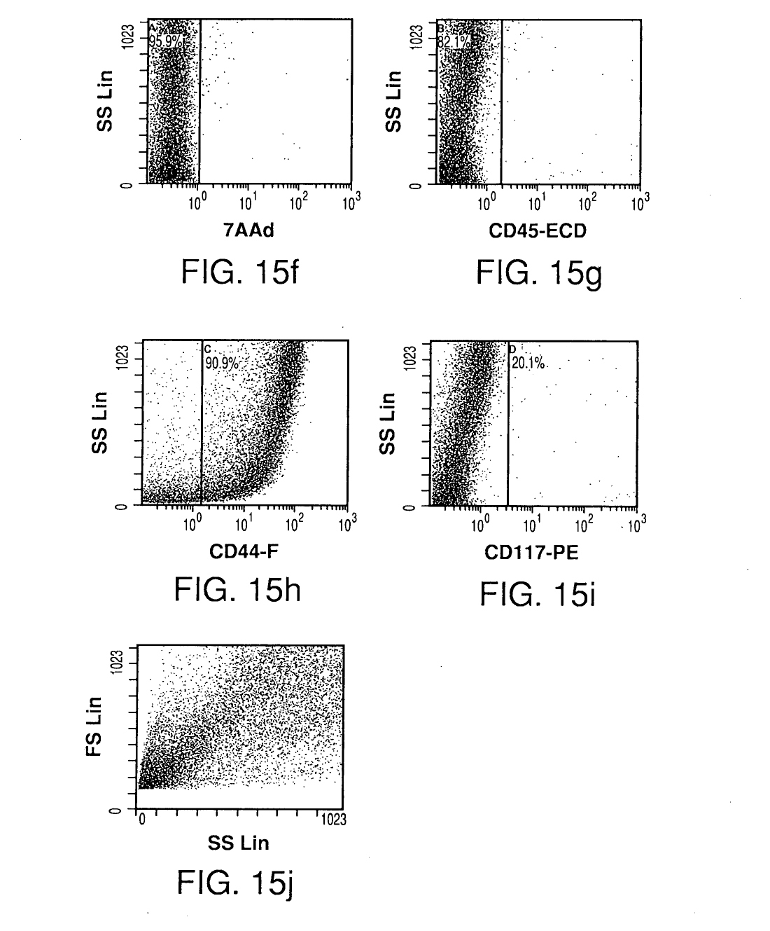

Phenotype analysis was performed in accordance with the methods described herein. The phenotype analysis was performed at different passages of the cell culture for cell surface markers including CD29, CD34, CD44, CD45, CD90, CD 105, CD117 and CD166. The cell phenotyping was performed with commercially-available monoclonal and polyclonal antibodies specific for the aforementioned cell surface markers using suitable flow cytometry analysis. As summarized in Table 6 and at various passages, the data collected from the assessment showed that the cells of Cell Line PLF05 expressed CD29, CD44, CD90, CD105, CD117 and CD166 with a high percentage of viability and low or no CD44 throughout the duration of cell culture. The results of the phenotyping and validity assessment are shown in FIGS. 15a through 15j.

TABLE 6
Phenotype and Validity Analysis of Cell Line PLF05

| | | CD44- | CD45- | | | | | | | |
|---|---|---|---|---|---|---|---|---|---|---|
| | CD117- | FITC | ECD | 7AAD- | 7AAD- | | | | | |
| Passage # | PE | (POS) | (NEG) | TEST | ISO | CD166 | CD105 | CD29 | CD34 | CD90 |
| PLF05-P5 | 3.30% | 90.60% | 93.30% | 98.40% | 98.30% | N/A | N/A | N/A | N/A | N/A |
| PLF05- | 5.40% | 93.80% | 85.60% | 97.70% | 98.20% | 96.00% | 97.50% | 90.80% | 8.50% | 28.30% |
| NEG | | | | | | | | | | |
| PLF05- | 10.50% | 86.90% | 88.00% | 95.90% | 95.00% | 87.70% | 95.70% | 89.50% | 7.00% | 30.30% |
| THAW P6 | | | | | | | | | | |
| PLF05- | 16.80% | 81.70% | 82.10% | 95.90% | 97.30% | 89.90% | 82.30% | 85.10% | 10.30% | 19.10% |
| POSFRAC | | | | | | | | | | |
| P6 | | | | | | | | | | |

Cell Line PLF10

A whole mammalian placenta was delivered and a tincture of iodine was placed on the fetal side of the placenta in accordance with the invention. Two pieces of placental tissue measuring about 6 cm by about 12 cm square and cut through the depth of the organ from fetal side to maternal side were obtained from the whole placenta through use of the sterile scalpel and forceps provided in a tissue collection kit. The DPBS of the tissue collection kit was poured into the sterile container of the tissue collection kit and the piece of placental tissue was submerged in the DPBS. The sterile container was closed, chilled and packaged for shipment in accordance with the present invention and then shipped to the processing facility. The piece of placental tissue arrived at the processing facility within about 15 hours after delivery of the whole placenta and collection of the pieces of placental tissue. The sterile container with the tissue sample was received at the processing facility and unpackaged from the shipping box and logged into the batch record.

The sterile container containing the DPBS and piece of placental tissue was disinfected, transferred into a clean room, and placed on ice in an ice pan. The sterile container was placed in a BSC and the top of the sterile container was removed. A sample of the DPBS buffer (about 4 ml) was removed from the sterile container using a sterile syringe and used to inoculate a BacT/ALERT blood culture bottles for detecting aerobic and anaerobic bacteria. The blood culture bottles were incubated at about 37° C. in a BacT/ALERT system for about 7 days, whereby the results indicated a positive identification for Enterococcus faecalis as shown in Table 7.

TABLE 7
BacT/ALERT System Analysis

| Status | Type | Loaded |
|---|---|---|
| POSITIVE - Enterococcus faecalis | BTA PF | Jan. 25, 2007 @ 21:46 |
| POSITIVE - Enterococcus faecalis | BTA | Jan. 25, 2007 @ 21:46 |

The pieces of placental tissue were removed from the sterile container for decontamination. Prior to disinfection, the fetal placental membrane of the piece of placenta was physically separated from portions of the maternal membrane of the piece of placenta using forceps and a scalpel, and the portions of the maternal piece of the placenta were discarded. Decidual tissue was also scraped away from the amniochorionic tissue with a scalpel.

The placental tissue was then subjected to a disinfection process in the BSC. The placental tissue was initially rinsed in about 50 ml of DPBS and then was disinfected by dipping it into a container of about 150 ml of about 50% mixture of Betadine (10% Povidone-iodine Topical Solution—Purdue Products) and about 50% PBS buffer for about 5 seconds and removed. The placental tissue was then disinfected by dipping it in a container of about 150 ml of a solution of several antibiotics to kill a broad range of pathogens for about 3 minutes. The antibiotic solution comprised three antibiotics including Amphotericin B, Streptomycin, and Cephazolin (X-Gen Pharmaceuticals Inc.) mixed with a buffer HBSS. The antibiotic solution was prepared by suspending about 50 mg of each antibiotic in about 10 ml of HBSS. The suspended antibiotics were added to about 100 ml of HBSS. Finally, the placental membrane was rinsed by dipping it in DPBS (1× without Calcium and Magnesium—Cellgro) for about 5 seconds to about 15 seconds.

The disinfected pieces of placental tissue were then transferred to a sterile container with DPBS (1× without Calcium and Magnesium—Cellgro). All of the steps of tissue separation and decontamination were performed at room temperature of about 18° C.

The sterile container with disinfected placental tissue was placed on ice in preparation for performing the subsequent steps of the invention. The portions of the placental tissue that was removed or residual from tissue processing, the washes and antibiotic solutions were discarded as medical waste.

The placental tissue was removed from the sterile container and placed in a wash solution in a Petri dish. The wash solution comprised about 500 ml of HBSS without Calcium chloride and Magnesium chloride (Gibco), about 5 ml of Heparin (Heparin Sodium 1,000 USP Units/ml—American Pharmaceutical Partners), about 2.5 ml of DNase (Genentech 2.5 mg/ml), and about 50 ml of protein (25% Human serum albumin HSA—Telacris Biotherapeutics). The wash solution was chilled on ice. The placental tissue in the wash was minced with a scalpel and forceps to the smallest possible pieces, which were suspended in the wash solution in a Petri dish.

Enzyme digestion of the minced pieces of placental tissue occurred in sequential steps. About 22 grams of minced placental tissue was subjected to the sequential steps of enzymatic digestion according to the following process. About 6 grams of placental tissue was placed in a first digestion solution of about 10 ml in a first tube. The first tube was incubated in a sterile bag at about 37° C. in an incubator for about 10 minutes. The placental tissue was removed from the digestion solution in the first tube and then placed in a second tube with about 10 ml of new digestion solution. The digestion solution in the first tube was discarded. The second tube with digestion solution and placental tissue was incubated in a sterile bag at about 37° C. in an incubator for about 20 minutes. The tube was inverted at about 10 minutes. The placental tissue was removed from the second tube and placed in a third tube containing about 10 ml of new digestion solution. The enzymatic reaction in the enzyme digestion solution in the second tube was stopped by adding about 4 ml 25% HSA (Baxter with concentrations of 1000 units per ml) and mixing the digestion solution with the HSA. The second tube was placed on ice.

The third tube containing about 10 ml of digestion solution and placental tissue was incubated in a sterile bag at 37° C. in an incubator for about 20 minutes. The tube was inverted at about 10 minutes. The placental tissue was removed from the third tube and placed in a fourth tube containing 10 ml of digestion solution. The enzymatic reaction in the third tube was stopped by adding about 4 ml 25% HSA (Baxter with concentrations of 1000 units per ml) and mixing the digestion solution with the HSA. The third tube was placed on ice.

The fourth tube containing about 10 ml of digestion solution and placental tissue was incubated in a sterile bag at 37° C. in an incubator for about 20 minutes. The tube was inverted at about 10 minutes. The placental tissue was removed from the fourth tube and placed in a fifth tube containing about 10 ml of digestion solution. The enzymatic reaction in the fourth tube was stopped by adding about 4 ml 25% HSA (Baxter with concentrations of 1000 units per ml) and mixing the digestion solution with the HSA. The fourth tube was placed on ice.

The fifth tube containing about 10 ml of digestion solution and placental tissue was incubated in a sterile bag, at 37° C. in an incubator for about 20 minutes. The tube was inverted at about 10 minutes. The placental tissue was removed from the fifth tube and discarded. The enzymatic reaction in the fifth tube was stopped by adding about 4 ml 25% HSA (Baxter with concentrations of 1000 units per ml) and mixing the digestion solution with the HSA. The fifth tube was placed on ice.

The digestion solution containing placental cells in the second through fifth tubes were filtered through a 100 micron filter. About 10 ml of wash solution was used to wash and rinse the contents of the second through fifth tubes into the filter and to wash and rinse the filter. The filtered digestion solution and wash solution was collected in 50 ml collection tubes. The 50 ml collection tubes were centrifuged at about 2000 rpm for about 7 minutes at about 20° C. The supernatant was removed from the collection tubes and the pelleted cells were re-suspended in about 45 ml of wash solution. The re-suspended cells were filtered through a 100 micron filter and into a 50 ml collection tube. The filter was rinsed with wash solution. The filtered, re-suspended cells and wash solution were combined into a 50 ml collection tube and wash solution was added to bring the total volume up to about 35 ml. The collection tube was centrifuged at about 2000 rpm for about 7 minutes at about 20° C.

The placental cells in the wash solution collected in conical collection tubes were subjected to centrifugation to concentrate the placental cells. The wash solution containing placental cells were equally separated into conical tubes for centrifugation. Centrifugation comprised subjecting the placental cells suspended in the wash solution to about 2000 rpm for about 7 minutes between about 20° C. The conical collection tubes were removed from the centrifuge and the supernatant is aspirated with suction and discarded. About 2 to about 3 ml of wash solution was used to re-suspend the pelleted cells in each tube. All of the re-suspended placental cells in the wash solution were transferred into a 50 ml conical collection tube, and the volume of the cellular suspension in the conical collection tube was brought up to a total volume of about 31 ml with the wash solution. About 100 ul of the cellular suspension was removed for testing the total nucleated cell count and viability with Trypan blue.

The cellular suspension was concentrated through centrifugation. The suspension was centrifuged in the conical collection tube at about 2000 rpm for about 7 minutes at about 20° C.

Bacteriological analysis of the supernatant was performed using the BacT/ALERT system for microbial analysis. A new sterile alcohol pad was opened and placed on top of the septum of the culture bottle. A sterile syringe used to collect between about 4 ml of supernatant from the conical collection tube after centrifugation and to inoculate the BacT/ALERT blood culture bottles for detecting aerobic and anaerobic bacteria. The blood culture bottles were incubated at about 37° C. in a BacT/ALERT system for about 7 days, whereby the results were negative as shown in Table 8.

TABLE 8
BacT/ALERT System Analysis

| Status | Type | Loaded |
|---|---|---|
| NEGATIVE | BTA PF | Jan. 25, 2007 @ 21:46 |
| NEGATIVE | BTA | Jan. 25, 2007 @ 21:46 |

The pelleted cells were re-suspended with wash solution up to about 6 ml. About one ml of cellular suspension was removed and placed into a post-processing tube and was tested for the total nucleated cell count and cell viability.

About 5 ml of suspension was prepared for cryopreservation. The about five ml of cellular suspension obtained by centrifugation was combined with a cryopreservation agent in preparation for cryopreservation. The cryopreservation agent comprised about 3 ml of the buffer DPBS, about one ml of the protein HSA (Telacris Bio), and about one ml of the preservative DMSO (99% Stemsol). The cryopreservation agent was carefully added to the about 5 ml of cellular suspension to a total volume of about 10 ml of mixture of cellular suspension and cryopreservation agent. The mixture was separated into two 5 ml aliquot volumes in cryovials in several vials adapted for cryopreservation.

The cryovials with the placental cells and cryopreservation agent were subjected to several temperature reduction steps to reduce the temperature of the placental cells comprising fetal placental cells to a final temperature of about −90° C. with a controlled rate freezer (Cryomed Thermo Form a Controlled Rate Freezer 7454 (Thermo Electron, Corp.)). The following temperature reduction steps were programmed in the controlled rate freezer: first reducing the mixture of the placental cells and cryopreservation agent to about 4° C. and then reducing the mixture at about 1° C. per minute to about −3° C., and then about 10° C. per minute to about −20° C., and then about 1° C. per minute to about −40° C., and finally about 10° C. per minute to about −90° C. The cryovials containing the mixture of the population of cells and cryopreservation agent were placed in the controlled rate freezer and subjected to the temperature reduction steps. Once the mixture and cryopreservation agent reached about −90° C., the cryopreservation vials were transferred to a cryogenic storage unit and stored in the vapor of liquid Nitrogen at a temperature at or below about −135° C. in a LN2 Freezer MVE 1830 (Chart Industries).

The cryopreserved placental cells were prepared for cell culture after being thawed. The thawing step comprised preparing aliquots of about 15 ml of density gradient media (Histopaque—Sigma-Aldrich) at about room temperature for each vial containing about 5 ml of cryopreserved cells to be thawed; and then preparing about 25 ml aliquots of Chang's complete media for each vial containing about 5 ml of cryopreserved cells to be thawed.

The cryopreserved cells were thawed by removing the vials from the vapor phase of the liquid nitrogen storage freezer. The vials were placed in about a 37° C. to 40° C. water bath and agitated. The cells were not allowed to completely thaw, but the vials contained ice. The thawed cells were diluted by placing the about 5 ml aliquot into the about 25 ml aliquot of chilled Chang's complete media containing about one mg of DNAse available through Pulmozyme.

The diluted cell suspension was mixed by inversion. The suspension was centrifuged at about 840 g for about 7 minutes at about 4° C. The supernatant was aspirated while not disturbing the pellet. The pellet was brought up to a total volume of about 30 ml with Chang's complete media. A small amount of the cell suspension was removed for analysis that includes cell count with a hemocytometer and viability testing using trypan blue. The about 30 ml suspension was overlaid on a density gradient solution (Histopaque—Sigma-Aldrich) and was centrifuged at about 420 g for about 30 minutes without a brake. The tube was removed from the centrifuge without disrupting the buffy coat. The supernatant was aspirated and the buffy coat was collected. The buffy coat was brought up to about 20 ml with Chang's complete media and was washed at about 840 g for about 7 minutes at about 4° C. The supernatant was aspirated, and the pellet was suspended in Chang's complete media up to about 10 ml. An aliquot of the suspension about 100 ul, was removed to perform a cell count and viability analysis.

The cells in suspension were seeded at about 4950 cells/cm² into an untreated tissue culture flask in Chang's complete media. The flask was incubated in about 5% CO₂ in a CO₂ incubator available through (Thermo Electron Corp.) at a temperature of about 37° C. The cell cultures were monitored for turbidity and pH change.

The flask was incubated initially for about 7 days or until the media was significantly out of range as noted by the color of the phenol red indicator in the media. All of the media was changed to maintain continuous factors that have been excreted into the media by the cells. After the media change at day 7, the cells became confluent by day 11 with a few small areas yielding 70% confluence. The cell culture went through several passages in accordance with the present invention.

CD117 placental cells were collected from the cell culture at passage 4 of the cell culture. In order to collect the CD117 placental cells, adherent cells were dissociated from the flask. The flask was rinsed with about 5 ml of Phosphate Buffered Saline (PBS) without calcium or magnesium. The PBS was removed from the flask with attached cells washed at least once. About one ml of a Trypsin-like recombinant enzyme (TrypLE™ Express—Gibco) was added to the cell culture in the flask. The flask was agitated to coat the cells with the enzyme. The flask with enzyme was incubated for about 5 minutes at about 37° C. After incubation, the flask was gently tapped on a solid surface to dislodge the cells. The flask was diluted with about 2 ml of Chang's complete media, and the cells transferred to a 15 ml centrifuge tube for washing with Chang's complete media. The tube was centrifuged for about 7 minutes at about 100 g at about 4° C. The supernatant was aspirated and discarded. The pellet was suspended in a suitable volume of Chang's complete media.

The cellular suspension was centrifuged at about 300 g for about 10 minutes at about 4° C. The pellet was suspended in a working buffer with anti-human CD117 antibodies. The working buffer comprised PBS at about pH 7.2, bovine serum albumin, EDTA and about 0.09% Azide (or suitable replacement) (BD Biosciences). The pellet was suspended in about 100 ul of working buffer and about 5 ug of purified monoclonal mouse anti-human CD117 antibody (available as 104D2 from Santa Cruz or YB5.58 from BD Biosciences).

The solution comprising the cells, working buffer and anti-CD117 antibodies were incubated for an incubation period of about 20 minutes to about 25 minutes on ice. After the incubation period, the solution with the cells was washed with working buffer to remove unbound antibody and then centrifuged at about 300 g for about 10 minutes at about 4° C. After centrifugation, the supernatant was aspirated and was saved for analysis, and the pellet was suspended in working buffer.

A second batch of antibodies having microbeads affixed thereto and having an affinity for the goat anti-mouse IgG antibody were added to the working buffer used to suspend the pellet. The microbeads comprised iron oxide and polysaccharide (Miltenyi Biotec-130-048-401). A 2 ml vial of the aforementioned goat anti-mouse IgG was used for approximately 1.0×10e9 of total un-separated cells.

The cellular suspension was incubated for a second incubation period of about 30 minutes. After the incubation period, the cells were washed with working buffer, and the cells were centrifuged for about 10 minutes at about 300 g at about 4° C. The supernatant was aspirated and saved for analysis, and the pellet containing cells was suspended in about 500 ul of working buffer.

The CD117 stem cells were separated from the cellular suspension in working buffer using a MS column (Miltenyi Biotec) to separate the CD117 placental cells. The MS column was prepared by rinsing it with working buffer. For example, the volume of working buffer used to rinse the column may be about 500 ul. The column was placed in a magnetic field of a MACS separator (Miltenyi Biotec).

Unlabeled cells flowed through the column and were collected in a sterile tube for cell phenotyping and cell count. The unlabeled cells were identified as a negative fraction. The column was washed with working buffer after adding the cellular suspension.

The labeled CD117 placental cells were collected from the column after the column was washed. The labeled CD117 cells were collected by placing a sterile tube under the column and removing the column from the magnetic field. Once the column was removed from the magnetic field, the labeled CD117 placental cells passed through the column and into the sterile tube. Residual labeled CD117 placental cells in the column were washed out by adding working buffer to the column to wash the cells through the column and by stripping the column with a plunger to release the cells. The collected labeled CD117 placental cells were identified as the positive fraction. The positive fraction was centrifuged at about 300 g for about 10 minutes at about 4° C. and the supernatant aspirated. The pellet was suspended in about 5 ml of working buffer.

The positive fraction and the negative fraction are analyzed with a hemocytometer to obtain a total count of viable cells. The negative fraction was analyzed by flow cytometry for phenotyping. The TNC of the positive fraction was about 75,000 cells with about 95% viability by trypan blue. The TNC of the negative fraction was about 16 million cells with 100% viability by trypan blue.

The cells in suspension were seeded at about 3000 cells/cm² into an untreated tissue culture flask in Chang's complete media. The flask was incubated in about 5% CO₂ in a CO₂ incubator (Thermo Electron Corp.) at a temperature of about 37° C. The cell cultures were monitored for turbidity and pH change.

The flask was incubated initially for about 3 days. All of the media was changed to maintain continuous factors that have been excreted into the media by the cells. After the media change at day 3, the cells became confluent by day 4. The cell culture went through several passages according to the methods of the present invention.

CD117 placental cells were collected from the cell culture at passage 1. In order to collect the CD117 placental cells, adherent cells were dissociated from the flask. In order to dissociate the cells from the flask, the media was aspirated via an automated pipette. The flask was rinsed with about 5 ml of Phosphate Buffered Saline (PBS) without calcium or magnesium. The PBS was removed from the flask with attached cells that have been washed at least once. About one ml of a Trypsin-like recombinant enzyme such as TrypLE™ Express available through Gibco was added, pre-warmed at about 37° C., to the cell culture in the flask. The flask was agitated to coat the cells with the enzyme. The flask with enzyme was incubated for about 5 minutes at about 37° C. After incubation, the flask was gently tapped on a solid surface to dislodge the cells. The flask was diluted with about 2 ml of Chang's complete media, and the cells transferred to a 15 ml centrifuge tube for washing with Chang's complete media. The tube was centrifuged for about 7 minutes at about 100 g at about 20° C. The supernatant was aspirated and discarded. The pellet was suspended in a suitable volume of Chang's complete media.

The cell culture containing CD117 fetal cells were prepared for cryopreservation. About one ml of human serum albumin, about 3 ml of DPBS and about one ml of DMSO were added to the about 5 ml of the positive fraction. The solution containing CD117 placental cells is mixed and cooled on ice for about 10 minutes. About one ml of DMSO was added as a cryopreservative. The resulting solution was aliquoted into cryovials. The cryovials were then cryopreserved in a controlled rate freezer (Cryomed) in accordance with controlled rate freezer protocol of the present invention as described herein in further detail. Once the solution containing CD117 placental cells reached the target temperature of about −90° C., the cryovials were transferred into a long term storage freezer and stored at about −135° C. or less.

Phenotype and Validity Analysis of Cell Lines PLF10

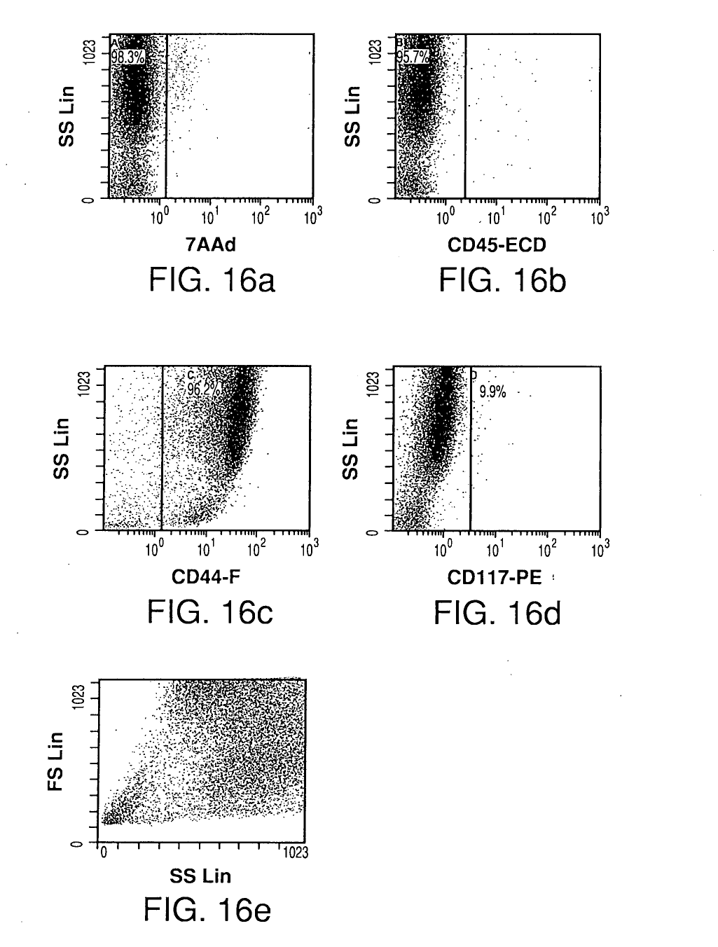

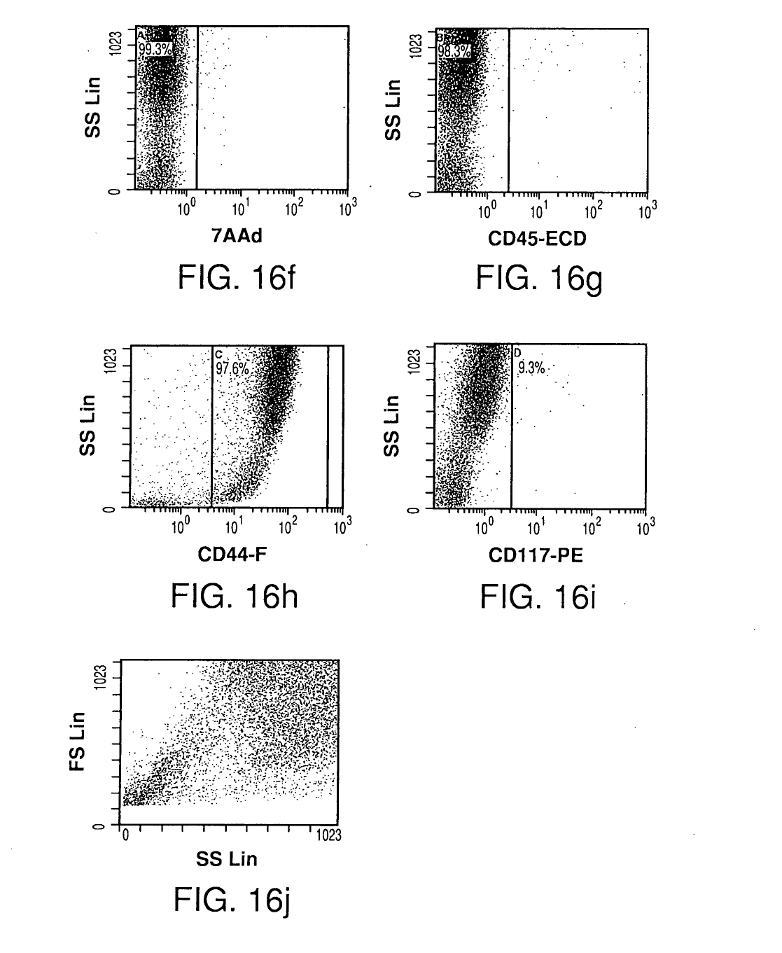

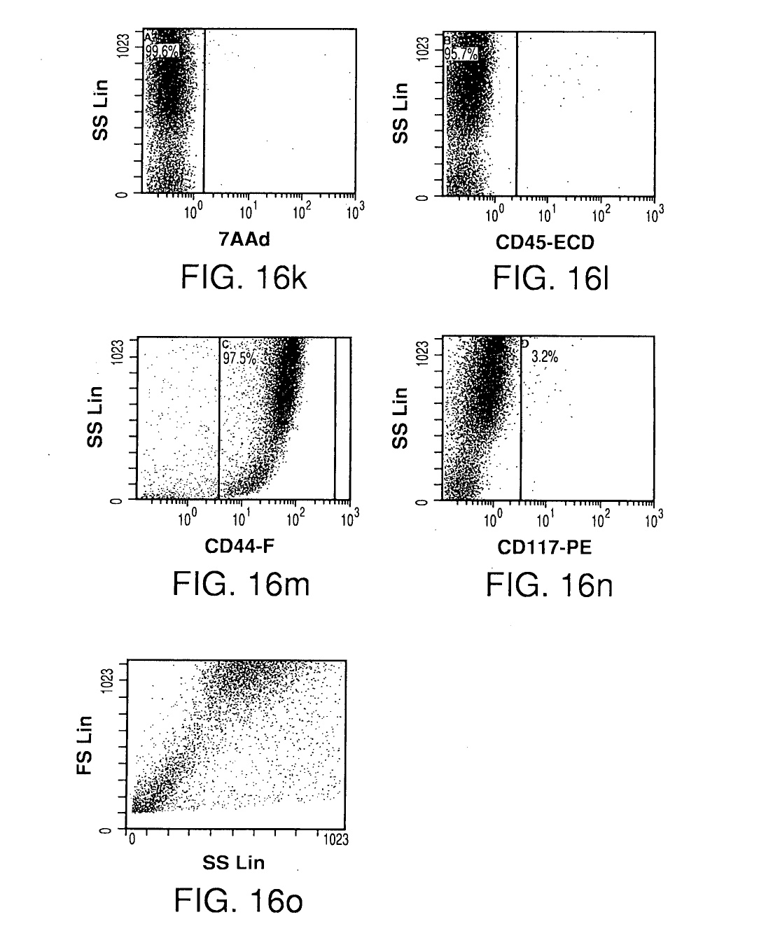

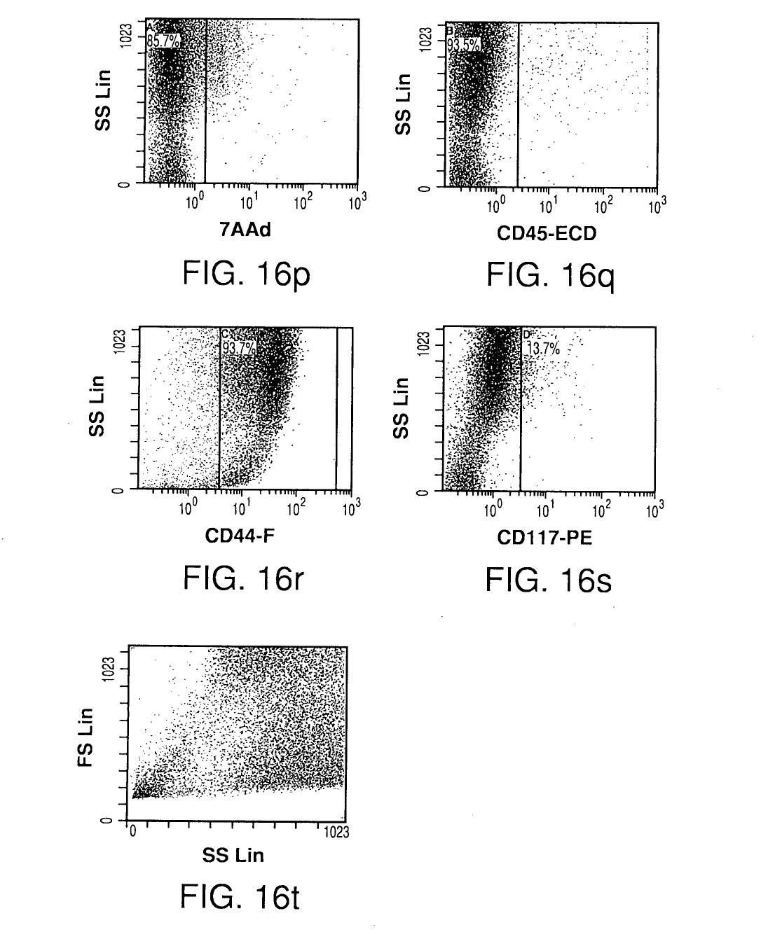

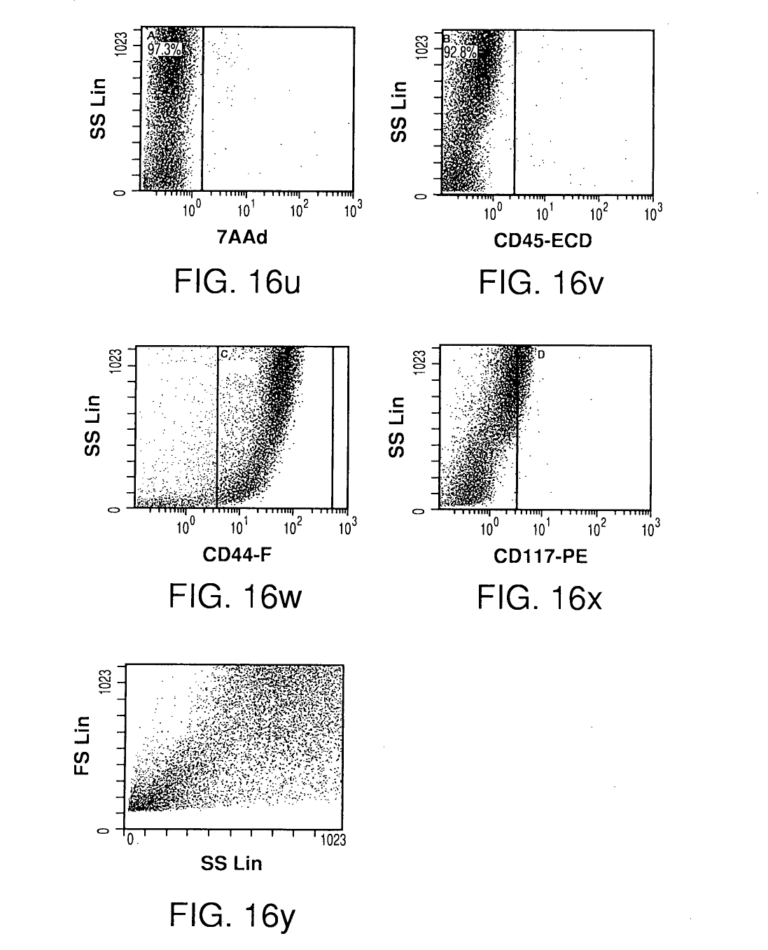

Phenotype analysis was performed on the cells of Cell Line PLF10 at several points during cell culture in accordance with the methods of the present invention. The phenotype analysis was performed for cell surface markers including CD29, CD34, CD44, CD45, CD90, CD 105, CD117 and CD166. The cell phenotyping was performed with commercially-available monoclonal and polyclonal antibodies specific for the aforementioned cell surface markers using suitable flow cytometry analysis. At various passages and as summarized in Table 9, the data collected from the assessment showed that the cells of Cell Line PLF10 expressed CD29, CD44, CD90, CD105, CD117 and CD166 with a high percentage of viability and low or no CD45 expression through out the cell culture. The results of the phenotyping and validity assessment are shown in FIGS. 16a through 16j.

TABLE 9
Phenotype and Validity Analysis of Cell Line PLF10

| | | CD44- | CD45- | | | | | | | |
|---|---|---|---|---|---|---|---|---|---|---|
| | CD117- | FITC | ECD | 7AAD- | 7AAD- | | | | | |
| Passage # | PE | (POS) | (NEG) | TEST | ISO | CD166 | CD105 | CD29 | CD34 | CD90 |
| PLF10- | 2.40% | 95.10% | 99.40% | 99.00% | 99.30% | 98.60% | 97.00% | 96.40% | 0.20% | 91.10% |
| NEG | | | | | | | | | | |
| PLF10- | 0.00% | 98.10% | 99.80% | 99.70% | 99.70% | 98.90% | 98.80% | 98.10% | 0.60% | 92.40% |
| THAW | | | | | | | | | | |
| P4 | | | | | | | | | | |
| PLF10- | 1.30% | 97.50% | 98.80% | 99.70% | 99.70% | 97.90% | 97.90% | 97.30% | 3.90% | 93.20% |
| THAW | | | | | | | | | | |
| P5 | | | | | | | | | | |
| PLF10- | 4.30% | 97.70% | 99.70% | 97.50% | 99.90% | N/A | N/A | N/A | N/A | N/A |
| THAW | | | | | | | | | | |
| P6 | | | | | | | | | | |
| PLF10- | 9.00% | 96.10% | 95.70% | 98.30% | 99.30% | 96.20% | 96.50% | 94.30% | 6.70% | 95.20% |
| THAW | | | | | | | | | | |
| P8 | | | | | | | | | | |
| PLF10- | 2.50% | 97.20% | 98.50% | 97.10% | 99.70% | N/A | N/A | N/A | N/A | N/A |
| THAW | | | | | | | | | | |
| P9 | | | | | | | | | | |
| PLF10- | 8.90% | 97.50% | 98.30% | 99.30% | 99.80% | 98.20% | 92.50% | 94.60% | 1.50% | 91.00% |
| THAW | | | | | | | | | | |
| P12 | | | | | | | | | | |
| PLF10- | 4.20% | 97.50% | 97.70% | 99.50% | 99.90% | 89.70% | 90.00% | 88.50% | 4.90% | 96.40% |
| THAW | | | | | | | | | | |
| P15 | | | | | | | | | | |
| PLF10- | 8.80% | 93.80% | 95.70% | 95.90% | 99.30% | N/A | N/A | N/A | N/A | N/A |
| THAW | | | | | | | | | | |
| P16 | | | | | | | | | | |
| PLF10- | 10.40% | 93.40% | 92.30% | 97.30% | 98.30% | N/A | N/A | N/A | N/A | N/A |
| THAW | | | | | | | | | | |
| P18 | | | | | | | | | | |
| PLF10- | 11.60% | 94.70% | 95.60% | 97.90% | 99.50% | N/A | N/A | N/A | N/A | N/A |
| THAW | | | | | | | | | | |
| P21 | | | | | | | | | | |
| PLF10- | 17.40% | 94.20% | 94.40% | 97.80% | 99.20% | N/A | N/A | N/A | N/A | N/A |
| THAW | | | | | | | | | | |
| P22 | | | | | | | | | | |
| PLF10- | 9.10% | 94.60% | 92.80% | 97.30% | 98.60% | 90.70% | 86.90% | 89.00% | 4.40% | 97.40% |
| THAW | | | | | | | | | | |
| P23 | | | | | | | | | | |
| PLF10- | 15.30% | 95.00% | 93.20% | 97.50% | 97.60% | N/A | N/A | N/A | N/A | N/A |
| THAW | | | | | | | | | | |
| P25 | | | | | | | | | | |
| PLF10- | 8.30% | 93.00% | 95.10% | 96.60% | 98.90% | N/A | N/A | N/A | N/A | N/A |
| THAW | | | | | | | | | | |
| P27 | | | | | | | | | | |

Phenotype analysis was performed at different passages of the cell culture of cells immunoselected for CD117 immunoselected cells at passage 4 of the aforementioned cell culture. Phenotype analysis was performed in accordance with the methods described herein. The phenotype analysis was performed at different passages of the cell culture for cell surface markers including CD29, CD34, CD44, CD45, CD90, CD 105, CD117 and CD166. The cell phenotyping was performed with commercially-available monoclonal and polyclonal antibodies specific for the aforementioned cell surface markers using suitable flow cytometry analysis. At various passages and as summarized in Table 10, the data collected from the assessment showed that the cells of Cell Line PLF10 expressed CD29, CD44, CD90, CD105, CD117 and CD166 with a high percentage of viability and low or no CD45 expression throughout the duration of cell culture. The results of the phenotyping and validity assessment are shown in FIGS. 16k through 16x.

TABLE 10
Phenotyping and Validity Analysis for CD117 Positive Fraction

| | | CD44- | CD45- | | | | | | | |
|---|---|---|---|---|---|---|---|---|---|---|
| | CD117- | FITC | ECD | 7AAD- | 7AAD- | | | | | |
| Passage # | PE | (POS) | (NEG) | TEST | ISO | CD166 | CD105 | CD29 | CD34 | CD90 |
| PLF10- | 0.20% | 97.90% | 98.90% | 99.50% | 99.80% | 98.60% | 97.80% | 97.70% | 0.70% | 94.00% |
| POS P3 | | | | | | | | | | |
| PLF10- | 3.00% | 97.40% | 98.50% | 99.60% | 99.90% | 98.30% | 98.00% | 97.50% | 1.60% | 94.30% |
| POS P7 | | | | | | | | | | |
| PLF10- | 4.90% | 97.40% | 98.50% | 99.60% | 99.90% | N/A | N/A | N/A | N/A | N/A |
| POS P8 | | | | | | | | | | |
| PLF10- | 4.90% | 96.80% | 97.90% | 99.30% | 99.90% | 97.10% | 98.10% | 96.30% | 1.90% | 96.40% |
| POS P9 | | | | | | | | | | |
| PLF10- | 12.30% | 93.50% | 93.50% | 85.70% | 98.40% | 93.00% | 85.80% | 85.70% | 6.50% | 89.10% |
| POS | | | | | | | | | | |
| P10 | | | | | | | | | | |
| PLF10- | 15.60% | 94.90% | 95.50% | 98.30% | 99.10% | 94.70% | 95.20% | 92.40% | 2.70% | 96.10% |
| POS | | | | | | | | | | |
| P11 | | | | | | | | | | |
| PLF10- | 9.40% | 95.50% | 94.30% | 98.10% | 98.90% | N/A | N/A | N/A | N/A | N/A |
| POS | | | | | | | | | | |
| P13 | | | | | | | | | | |
| PLF10- | 12.60% | 94.70% | 91.60% | 97.40% | 98.80% | N/A | N/A | N/A | N/A | N/A |
| POS | | | | | | | | | | |
| P14 | | | | | | | | | | |
| PLF10- | 5.40% | 91.50% | 93.80% | 98.00% | 98.80% | N/A | N/A | N/A | N/A | N/A |
| POS | | | | | | | | | | |
| P16 | | | | | | | | | | |
| PLF10- | 13.60% | 92.80% | 94.10% | 96.90% | 99.00% | N/A | N/A | N/A | N/A | N/A |
| POS | | | | | | | | | | |
| P17 | | | | | | | | | | |
| PLF10- | 12.50% | 92.20% | 93.90% | 97.70% | 99.10% | N/A | N/A | N/A | N/A | N/A |
| POS | | | | | | | | | | |
| P18 | | | | | | | | | | |
| PLF10- | 9.10% | 91.80% | 95.40% | 97.70% | 97.40% | 89.80% | 86.40% | 86.00% | 1.90% | 89.30% |
| POS | | | | | | | | | | |
| P19 | | | | | | | | | | |
| PLF10- | 5.40% | 90.60% | 93.60% | 95.10% | 98.00% | N/A | N/A | N/A | N/A | N/A |
| POS | | | | | | | | | | |
| P23 | | | | | | | | | | |

Genotype Analysis of Cell Line PLF10

Human Identification-Multiplex Short Tandem Repeat (STR) Analysis was performed on the Cell Line PLF10 and on reference samples of maternal peripheral blood and baby cord blood collected at delivery to determine the origin of the cells of Cell Line PLF10.

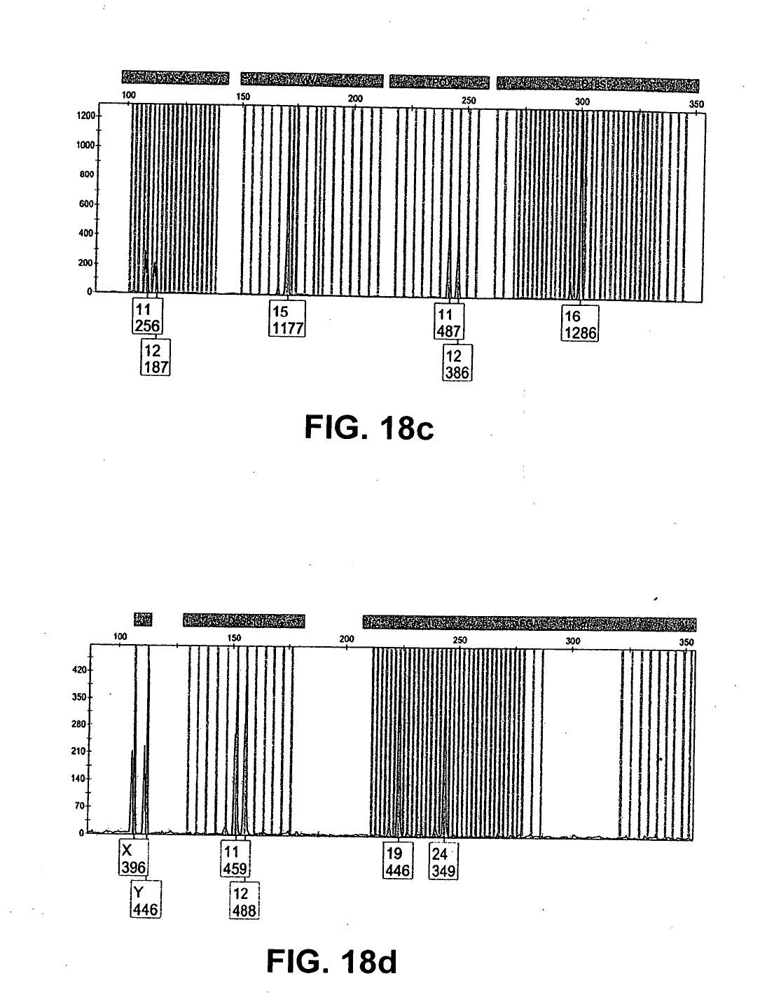

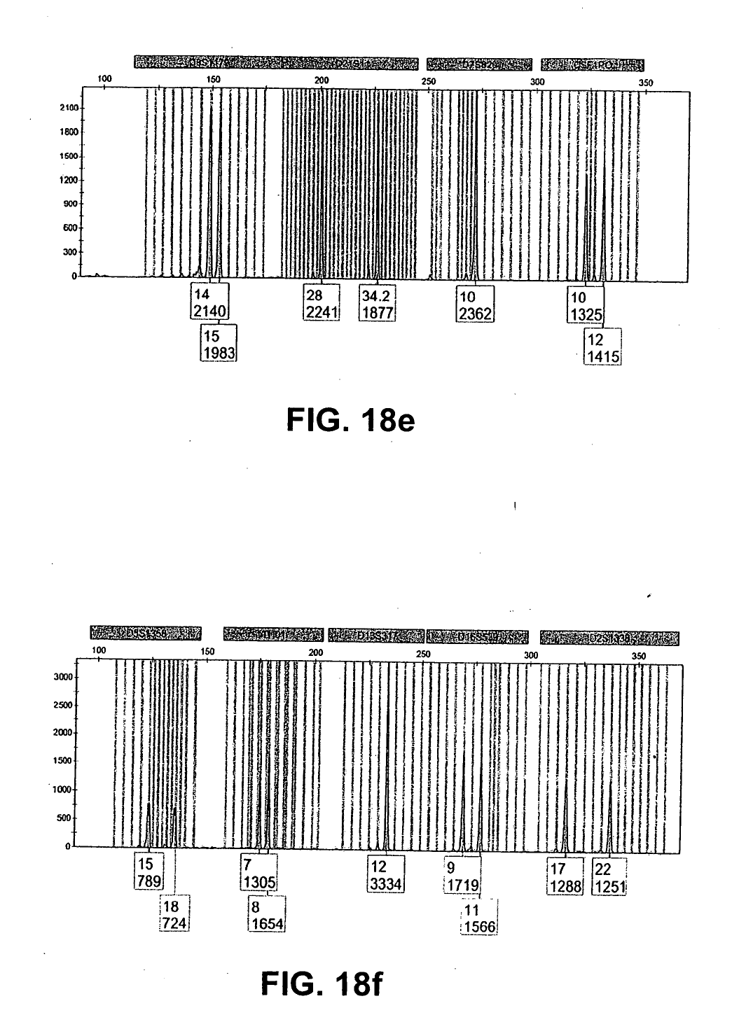

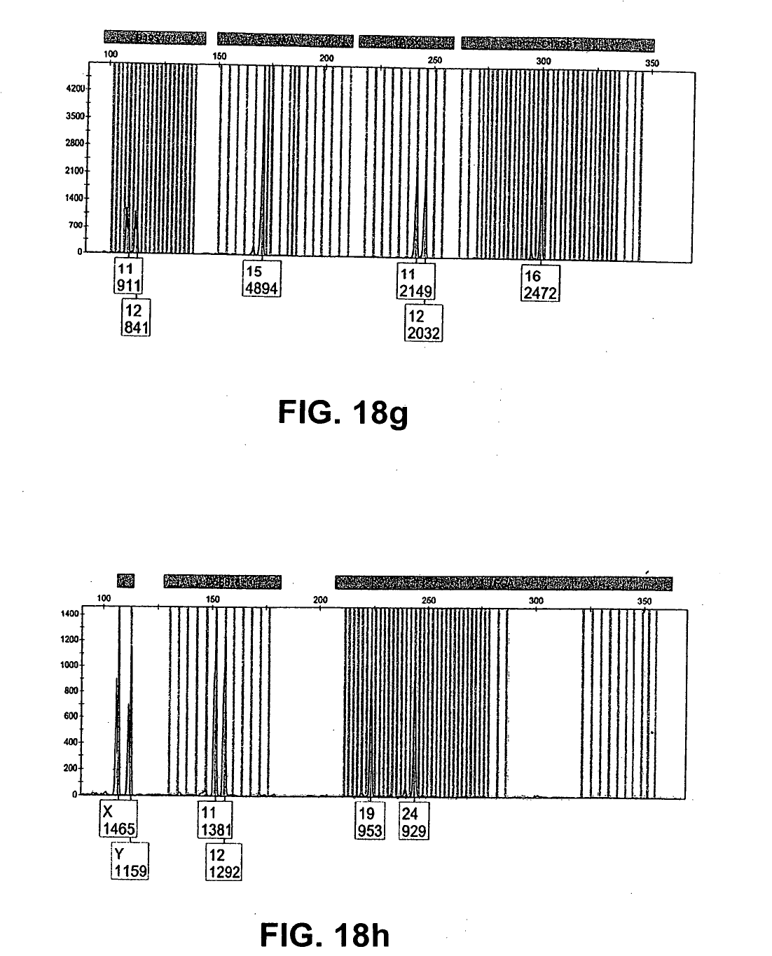

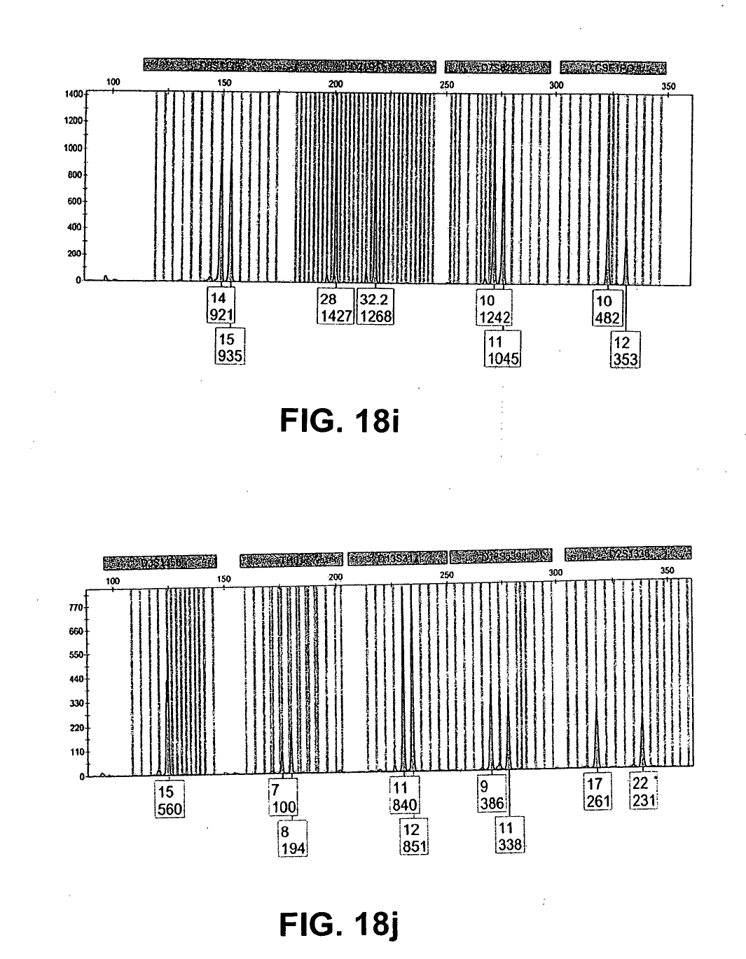

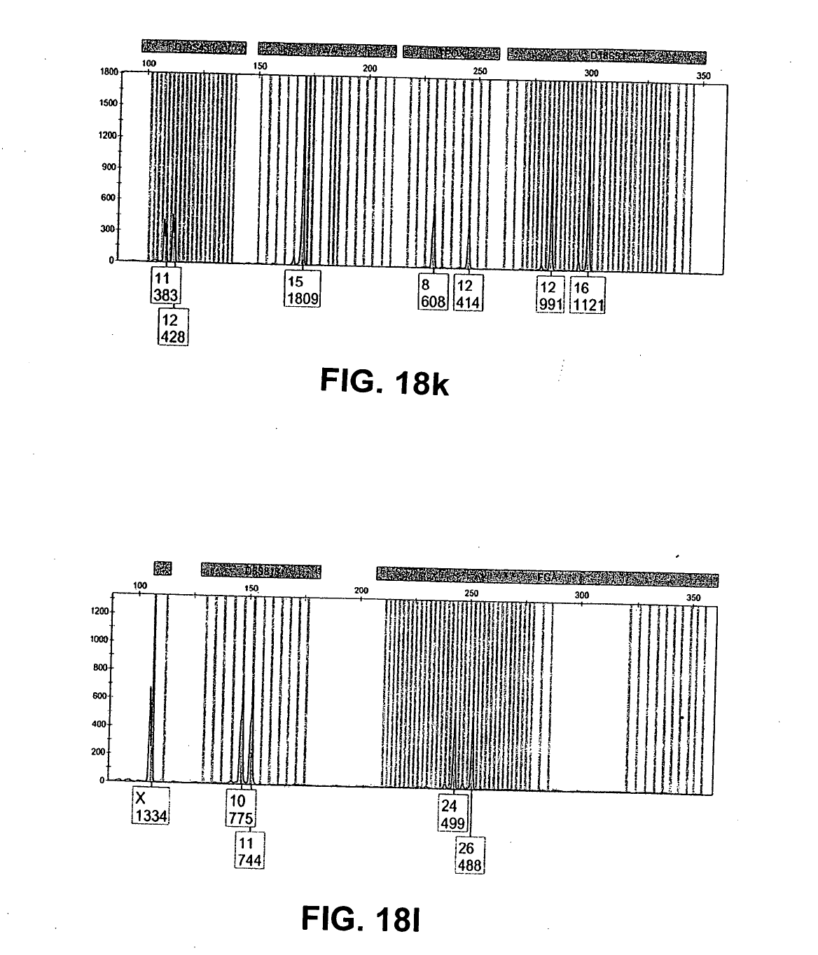

The STR Analysis involved investigating 15 different short tandem repeat (STR) gene regions plus amelogenin on the X and Y chromosomes were simultaneously subjected to PCR and then analyzed. Four separate fluorescent dye labels were used to label the samples. The dyes were coupled to PCR primers. Each of these fluorescent dyes emitted its maximum fluorescence at a different wavelength, that was detected by an analyzer. The 15 STR loci investigated were D8S1179, D21S11, D7S820, CSF1PO, D3S1358, TH01, D13S317, D16S539, D2S1338, D19S433, vWA, TPOX, D18S51, D5S818, FGA. The amplified product was electrophoresed on ABI 3100 Genetic Analyzer and analyzed using the GeneMapper ID software program. The sensitivity of the assay to detect mixed chimerism was about 5%. The results of the STR analysis indicated that the PLF10 specimen was of single individual origin and was 100% fetal and 0% maternal as shown in FIGS. 18a through 18l.

### DEFINITIONS

As used herein, the following terms shall have the definitions set forth below, unless the context in which such term is used suggests otherwise.

Amphotericin B (X-Gen) can be obtained at 50 mg/vial (Cardinal—#119140).

BSC means biological safety cabinet.

Cefazolin can be obtained at 1 gm/vial (Cardinal—#3455268).

Cm means centimeter.

Collagenase is an enzyme used to degrade collagen derived from Clostridium histolyticum.

CVS means Chorionic Villus Sampling is generally procured by a health care provider when they insert a small tube in through the vagina or abdomen to remove a small section of chorionic villi tissue from the placenta for prenatal diagnosis and karyotyping.

DMEM means Dulbecco's Minimal Essential Medium.

DMSO means Dimethyl sulfoxide.

DNase means Deoxyribonuclease used to break down DNA found after non-viable cells have lysed.

DPBS means Dulbecco's Phosphate Buffered Saline.

HBSS means Hank's balanced salt solution.

Heparin is a glycosaminoglycan having anticoagulant properties.

HSA means Human Serum Albumin which is an abundant plasma protein that can act as a transporter protein.

IPA means Isopropyl Alcohol used for disinfection typically at about 70% concentration.

LSM means Lymphocyte Separation Media used to perform a density gradient cell separation.

μg means microgram.

μl, μL, ul and uL are used synonymously to mean microliter.

ml and mL are used synonymously to mean milliliter.

QC means Quality Control.

Streptomycin (X-Gen) can be obtained at 1 gm/vial (10) (Cardinal—#2833010).

X means multiply, i.e., by concentration or dilution.

Materials and Equipment

Materials for placental tissue collection kit may include, but are not limited to, placental tissue transport container; sterile tissue container—1 liter; Dulbecco's Phosphate Buffer Saline (DPBS); plastic zipped bags with absorbent towels; sterile scalpel and forceps; sterile ruler; tincture of Iodine and sterile gauze.

Processing materials for placental procurement by scalpel and forceps may include, but are not limited to, sterile scalpel; sterile disposable forceps; tincture of Iodine; DPBS (Mediatech or other suitable source) contains no calcium, magnesium or phenol red; sterile basin; sterile disposable gloves; sterile 4×4 gauze; sterile specimen container; and sterile gloves.

Processing materials for placental procurement by punch biopsy may include, but are not limited to, sterile punch biopsy; sterile disposable forceps; tincture of Iodine; DPBS (Mediatech or other suitable source) contains no calcium, magnesium or phenol red; sterile basin; sterile disposable gloves; sterile 4×4 gauze; sterile specimen container; and sterile gloves.

Tissue disinfecting materials may include, but are not limited to, DPBS (Mediatech or other suitable source) contains no calcium, magnesium or phenol red.—1×500 ml bottle; HBSS—1×500 ml bottle; 16-20 g luer lock needles—8; syringe—8; Cavicide; forceps—2; scissors—1; sterile disposable containers—4; blue ice pan; ice; disposable dipping containers—3; sterile disposable gloves; 4×4 gauze—1 package of autoclaved gauze; vacuum collection flask with associated tubing set; red biohazard bags in container; red biohazard sharps container; Betadine hospital grade 10%; Cefazolin, 1 gm/vial (Cardinal—#3455268 or other suitable source)—1 vial; Amphotericin B—X-Gen, 50 mg/vial (Cardinal—#119140 or other suitable source)—2 vials; Streptomycin—X-Gen, 1 gm/vial (10) (Cardinal—#2833010 or other suitable source)—2 vials; and IPA—isopropyl alcohol used for disinfection typically at about 70% concentration.

Tissue disinfecting equipment may include, but is not limited to, a biological safety cabinet (BSC) and automated pipettor.

Materials for placental cell isolation by enzyme digestion may include, but are not limited to, DPBS (Mediatech or other suitable source) contains no calcium, magnesium or phenol red; DNase, Pulmozyme (Genentech, Inc.); Heparin-preservative-free (American Pharmaceutical Partners Inc.) concentration 1000 Units per ml; Collagenase contains Class I and II—(Serva/Cresent Chemical)—either 500 mg NB-4 for research (cat# 17454.02) or 1 gram NB6 for GMP use (cat# 17458.01). Both may contain the same PZ activity > or =0.1 U/mg lyophilysate; Human Serum Albumin, 25% (Baxter healthcare Corporation, Glendale, Calif., USA or other suitable source); Cavicide; 70% Isopropyl alcohol; scissors; forceps; disposable scalpel; 50 ml tube rack; 5 ml tube rack; cell strainer—100 micron filter (BD); centrifuge inserts; blue ice pan; ice; alcohol wipes, about 70% Isopropyl alcohol; red top vacutainer tubes—5 ml; BacT/ALERT blood culture bottle; sterile 50 ml conicals; sterile Petri dish; 1 ml needle TB Syringe; 3 ml needle syringe; 16-20 g luer lock needles; 10 ml sterile pipette; sterile aspirating pipettes; 3 sterile transfer pipettes; sterile disposable gloves; 4×4 gauze; vacuum collection flask with associated tubing set; red biohazard bags in container; red biohazard sharps container; and specimen labels.

Equipment for placental cell isolation by enzyme digestion may include, but is not limited to, centrifuge with round buckets; centrifuge Inserts; biological safety cabinet (BSC); vacuum pump; inverted light microscope; scale; hemocytometer; 37° C. incubator; and automated pipettor.

Materials for placental cell isolation by mechanical separation may include, but are not limited to, DPBS (Mediatech) contains no calcium, magnesium or phenol red; Lymphocyte Separation Media (Mediatech cat. #25-072-CV); DNase, Pulmozyme (Genentech, Inc.); Heparin-preservative-free (American Pharmaceutical Partners Inc.) concentration 1000 units per ml; Human Serum Albumin, 25% (Baxter Healthcare Corporation, Glendale, Calif., USA or other suitable source); Cavicide; 70% Isopropyl alcohol; scissors; forceps; disposable scalpel; 50 ml tube rack; 5 ml tube rack; centrifuge inserts; cell strainer—100 micron filter (BD); blue ice pan; Ice; alcohol wipes, 70% Isopropyl alcohol; red top vacutainer tubes—5 ml; BacT/ALERT blood culture bottle; sterile 50 ml conicals; sterile Petri dish; 1 ml needle TB syringe; 3 ml needle syringe; 16-20 g luer lock needles; 10 ml sterile pipette; 3 sterile transfer pipettes; sterile disposable gloves; 4×4 gauze; vacuum collection flask with associated tubing set; red biohazard bags in container; red biohazard sharps container; and specimen labels.

Equipment for placental cell isolation by mechanical separation may include, but is not limited to, centrifuge with round buckets; centrifuge inserts; biological safety cabinet (BSC); vacuum pump; inverted light microscope; scale; and automated pipettor.

Processing materials may include, but are not limited to, DPBS (Mediatech or other suitable source) contains no calcium, magnesium or phenol red.—2×500 ml bottles; Lymphocyte Separation Media (Mediatech cat. #25-072-CV)—1×500 ml bottle; DNase, Pulmozyme (Genentech Inc.)—1×2.5 ml vial; Heparin-preservative-free (American Pharmaceutical Partners Inc) concentration 1000 Units per ml)—3×2 ml vials; Human Serum Albumin, 25% (Baxter Healthcare Corporation, Glendale, Calif., USA or other suitable source).—1 bottle; Cavicide; about 70% Isopropyl alcohol; forceps—3; disposable scalpel—1; 50 ml tube rack; 5 ml tube rack; 15 ml tube rack; centrifuge inserts; cell strainer—100 micron filter (BD)—16; blue ice pan; ice; alcohol wipes, about 70% Isopropyl alcohol; red top vacutainer tubes—5 ml×2; BacT/ALERT blood culture bottles; sterile 50 ml conicals—16; sterile 15 ml conicals—4; sterile Petri dish—2; 1 ml needle TB syringe—4; 3 ml needle syringe—4; 16-20 g luer lock needles—4; 10 ml sterile pipette—4; sterile aspirating pipettes—4; 3 sterile transfer pipettes—6; sterile disposable gloves; 4×4 gauze—1 package of autoclaved gauze; vacuum collection flask with associated tubing set; red biohazard bags in container; red biohazard sharps container; sterile steel basin—for sterile supplies; and specimen labels.

Processing equipment may include, but is not limited to, centrifuge with round buckets; centrifuge inserts; biological safety cabinet (BSC); vacuum pump; inverted light microscope; scale; and automated pipettor.

Cryopreservation materials may include, but are not limited to, DPBS; DMSO; 25% Human Serum Albumin; wash solution; bar-coded cryovial—1×5 ml; and QC vials—5×1 ml.

Cryopreservation equipment may include, but are not limited to, Planar cryopreservation freezer and liquid Nitrogen storage freezer.

Materials for flow cytometry may include, but are not limited to, FC500 Flow Cytometer; human placental cells within 48 hours of cell isolation; 5 uL to 100 uL micropipettor; Eppendorf pipettor; manual/electric pipettor; 1-200 uL pipette Tips; Plastibrand positive displacement tips (5.0 ml); serological pipettes, 5 ml and 25 ml; 12×75 mm polypropylene culture tubes; 50 ml tubes; test tube racks; distilled water; Isoflow Sheath fluid—stable at room temperature until expiration date on label, Do Not Freeze; and Coulter Clenz cleaning agent—store between about 2° C. to about 25° C., stable until expiration date or about 3 months after opening, remix by inversion if frozen and thawed.

Reagents for flow cytometry may include, but are not limited to, CD117-PE—stable to expiration date on vial when stored between about 2° C. to about 8° C. away from light, stable about 30 days after opening, watch for evidence of deterioration (change in color and/or clarity), bring to between about 20° C. to about 25° C. before use; CD44-FITC—stable to expiration date on vial when stored between about 2° C. to about 8° C. away from light, stable 30 days after opening, watch for evidence of deterioration (change in color and/or clarity), bring to between about 20° C. to about 25° C. before use; CD45-ECD—stable to expiration date on vial when stored between about 2° C. to about 8° C. away from light, stable 30 days after opening, watch for evidence of deterioration (change in color and/or clarity), bring to between about 20° C. to about 25° C. before use; IgG-FITC—stable to expiration date on vial when stored between about 2° C. to about 8° C. away from light, stable 30 days after opening, watch for evidence of deterioration (change in color and/or clarity), bring to between about 20° C. to about 25° C. before use; IgG-PE—stable to expiration date on vial when stored at between about 2° C. to about 8° C. away from light, stable 30 days after opening, watch for evidence of deterioration (change in color and/or clarity), bring to between about 20° C. to about 25° C. before use; IgG-ECD—stable to expiration date on vial when stored at between about 2° C. to about 8° C. away from light, stable 30 days after opening, watch for evidence of deterioration (change in color and/or clarity), bring to between about 20° C. to about 25° C. before use; 7-AAD viability dye—stable to expiration date on vial when stored between about 2° C. to about 8° C. away from light, stable 30 days after opening, watch for evidence of deterioration (change in color and/or clarity), bring to between about 20° C. to about 25° C. before use; Ammonium Chloride (NJ4CL) lysing solution 10× concentrated, stored at between about 2° C. to about 8° C., stable until expiration date, use working solutions at room temperature, discard at end of day; Human Serum Albumin 25%, store at between about 2° C. to about 8° C.; wash media comprising HBSS (Hanks with Ca+ and Mg+) 100 ml, 0.2 Heparin about 1 ml; HSA 25% about 10 ml; DNase about 20 drops; Kasumi-3 cell line—CD117+ cells; Stemtrol control cells —CD34− cells, stable to expiration date on vial when stored at between about 2° C. to about 8° C. away from light, stable 30 days after opening, watch for evidence of deterioration (change in color and/or clarity), bring to between about 20° C. to about 25° C. before use; timer; and vortex mixer.

The materials for cell culture may include but are not limited to a 37° C. water bath, hemocytometer, cover slips, lens paper, alcohol prep pads, 5 uL to 100 uL micropipettor, Eppendorf pipettor, manual/electric pipettor, 200 uL pipette tips, serological pipettes, 5 ml and 25 ml, 12×75 mm polypropylene culture tubes, 50 ml tubes, test tube racks, and untreated flasks.

Other suitable replacement reagents and products and manufacturers may be used in place of the specific reagents, products and manufacturers listed herein.

Modifications can be made to the embodiments described above without departing from the broad inventive concept thereof. Having described the preferred embodiments of the invention, additional embodiments, adaptations, variations, modifications and equivalent arrangements will be apparent to those skilled in the art. These and other embodiments will be understood to be within the scope of the appended claims and apparent to those skilled in the art.

What is claimed is:

1. A method for obtaining a population of cells enriched for fetal placental cells expressing CD117, comprising the steps of:
(a) disaggregating fetal placental tissue and separating the population of cells from disaggregated placental tissue;
(b) collecting and concentrating the population of cells; and
(c) cryopreserving the population of placental cells at or below about −135°.

2. A process for obtaining a population of placental cells enriched for fetal placental cells, comprising the steps of:
(a) procuring placental tissue from a whole placenta, the placental tissue comprising maternal tissue and fetal tissue;
(b) disaggregating the placental tissue comprising fetal tissue;
(c) isolating placental cells from disaggregated fetal tissue; and
(d) collecting the population of placental cells by concentrating the population of placental cells with at least one step of centrifugation.

3. A process for collecting placental cells expressing CD117 from placental tissue comprising fetal tissue, comprising the steps of;
(a) isolating the placental cells from the placental tissue;
(b) collecting and concentrating the placental cells; and
(c) cryopreserving the placental cells.

4. A system for collecting a population of cells enriched for fetal placental cells, comprising:
(a) a placental cell isolater, wherein the placental cell isolator disaggregates placental tissue comprising fetal tissue and separates placental cells from the disaggregate placental tissue;
(b) a placental cell collector, wherein the placental cell collector collects the placental cells separated from the disaggregate placental tissue:
(c) a placental cell concentrator, wherein the placental cell concentrator concentrates placental cells present in a suspension; and
(d) a placental cell cryopreserver, wherein the placental cell cryopreserver maintains the collected and concentrated placental cells at a temperature at or below about −135° C.

5. A process for isolating a population of fetal placental cells expressing CD117 from a population of placental cells, comprising the steps of:
(a) culturing a population of placental cells enriched for fetal placental cells;
(b) selecting placental cells expressing CD117 from a culture of the population of placental cells; and
(c) cryopreserving the placental cells expressing CD117.

6. A process for isolating a population of fetal placental cells from a population of placental cells comprising selecting placental cells expressing CD117 from a culture of the population of placental cells.

7. A process for isolating a population of fetal placental cells expressing CD117 from a population of placental cells, the process comprising,
(a) selecting placental cells expressing CD117 from a population of placental cells enriched for fetal placental cells;
(b) culturing the placental cells expressing CD117 selected from the population of placental cells enriched for fetal placental cells; and
(c) selecting placental cells expressing CD117 from a culture of placental cells.

8. A population of cells enriched for fetal placental cells obtained from the process comprising:
(a) culturing a population of cells comprising fetal placental cells; and
(b) selecting cells expressing CD117 from a culture of the population of cells.

9. A population of fetal placental cells obtained from the process comprising selecting placental cells expressing CD117 from a culture of a population of placental cells.

10. The population of fetal placental cells of claim 9, wherein the population expresses at least one of the cell surface markers CD29, CD44, CD73, CD90, CD105, CD117, CD166, SSEA-3 and SSEA-4

11. The population of fetal placental cells of claim 9, wherein the population has low or no expression of CD34, CD45 and CD133.

12. Fetal placental cells obtained from the process comprising
(a) selecting placental cells expressing CD117 from a population of placental cells comprising fetal placental cells;
(b) culturing the placental cells expressing CD117 selected from the population of placental cells comprising fetal placental cells; and
(c) selecting placental cells expressing CD117 from a culture of placental cells expressing CD117.

13. A population of cells enriched for fetal placental cells expressing at least one of the cell markers selected from the group consisting of CD29, CD44, CD73, CD90, CD105, CD117, CD166, SSEA-3 and SSEA-4.

14. A population of cells enriched for fetal placental cells expressing at least one of the cell markers selected from the group consisting of CD29, CD44, CD73, CD90, CD105, CD117, CD166, SSEA-3 and SSEA-4 and having low or no expression of at least one of the cell markers selected from the group consisting of CD34, CD45 and CD133.

15. A composition comprising a population of cells enriched for fetal placental cells and a preservation agent.

16. A composition comprising at least one fetal placental cell and a preservation agent.

17. A composition comprising at least one fetal placental cell expressing at least one of the cell markers selected from the group consisting of CD29, CD44, CD73, CD90, CD105, CD117, CD166, SSEA-3 and SSEA-4 and a preservation agent.

18. A composition comprising at least one fetal placental cell expressing at least one of the cell markers selected from the group consisting of CD29, CD44, CD73, CD90, CD105, CD117 and CD166 and having low to no expression of at least one of the cell markers selected from the group consisting of CD34, CD45 and CD133 and a preservation agent.

19. At least one fetal placental cell obtained from the process comprising:
(a) procuring placental tissue comprising maternal tissue and fetal tissue of a whole placenta;
(b) disaggregating the fetal tissue;
(c) isolating placental cells from disaggregated fetal tissue;
(d) collecting and concentrating placental cells in a population of cells;
(e) culturing the population of placental cells comprising fetal placental cells; and
(f) selecting placental cells expressing CD117 from a culture of the population of placental cells.

20. The process of claim 19, where the process comprises the further steps of:
(a) culturing the placental cells expressing CD117 selected from the population of placental cells comprising fetal placental cells; and
(b) selecting placental cells expressing CD117 from a culture of placental cells expressing CD117 comprising a population of fetal placental cells.

21. The process of claim 20, wherein the processes comprises the further step of cryopreserving the fetal placental cells at or below −135° C.
